(19) United States
(12) Patent Application Publication
(10) Pub. No.: US 2022/0235255 A1
(43) Pub. Date: Jul. 28, 2022

(54) FLUORINE SUBSTITUTED UNSYMMETRICAL ETHERS, AND COMPOSITIONS, METHODS AND USES INCLUDING SAME

(71) Applicant: HONEYWELL INTERNATIONAL INC., Charlotte, NC (US)

(72) Inventors: Sudharshanam Ramanathan, Charlotte, NC (US); Dimitrios Papanastasiou, Charlotte, NC (US); Haridasan K. Nair, Charlotte, NC (US)

(73) Assignee: HONEYWELL INTERNATIONAL INC., Charlotte, NC (US)

(21) Appl. No.: 17/569,490

(22) Filed: Jan. 5, 2022

Related U.S. Application Data
(60) Provisional application No. 63/134,156, filed on Jan. 5, 2021.
(60) Provisional application No. 63/145,502, filed on Feb. 4, 2021.
(60) Provisional application No. 63/215,174, filed on Jun. 25, 2021.

(51) Int. Cl. C09K 5/04; C09K 5/10; H01M 10/625; H01M 10/613; H01M 10/6567; H01M 10/0525; C07C 43/12

(52) U.S. Cl. CPC C09K 5/048; C09K 5/10; H01M 10/625; H01M 2220/20; H01M 10/6567; H01M 10/0525; C07C 43/123; H01M 10/613

(57) ABSTRACT

Compositions and methods comprising one or more compounds according to Formula I:

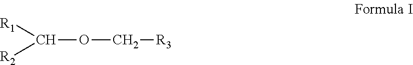

where
R₁, R₂ and R₃ are each independently CₓR′_{(2x+1)-y}H_{y};
each R′ is independently selected from F or Cl and wherein the value of (2x+1)−y is the total number of R′ substituents on the indicated carbon atom(s);
each x is independently equal to or greater than 1 and equal to or less than 6; and
y is equal to or greater than 0 and less than or equal to 2x+1, provided that the total number of R′ present in the compound is 6 or greater, and that the compound has from zero up to two (2) Cl substituents.

### CROSS REFERENCE TO RELATED APPLICATIONS

The present application is related to and claims the priority benefit of U.S. Provisional Application 63/134,156, filed Jan. 5, 2021, and of U.S. Provisional Application 63/145,502, filed Feb. 4, 2021, and of U.S. Provisional Application 63/215,174, filed Jun. 25, 2021, each of which is incorporated herein by reference in its entirety.

### FIELD

The present invention relates to fluorine substituted unsymmetrical ethers, to compositions containing same, and to methods and uses of these compounds and compositions in numerous applications, including electrolyte solvent for batteries (particularly lithium ion batteries), electrical insulation; electronics testing; etching fluids; solvent and carrier applications, fire protection, flammability suppression, blowing agent and heat transfer applications, including: temperature control in manufacture of electronic equipment; thermal management of operating electronic devices and power systems, and avionic and military cooling.

### BACKGROUND

There continues to be a need for inert fluorinated fluids which have low global warming potential while providing high thermal stability, low toxicity, nonflammability, good solvency, and a wide operating temperature range to meet the requirements of various applications. Those applications include, but are not restricted to: heat transfer, solvent cleaning, electrolyte compositions (including electrolyte solvents and additives) and fire extinguishing agents.

Applicants have come to appreciate that many challenging issues are associated with the development of new compounds and compositions for use in many important applications. In particular, applicants have come to appreciate the need for compositions, methods and systems which are at once environmentally acceptable (low GWP and low ODP), non-flammable, have low or no toxicity, and have excellent properties need for the particular application (for example, good solvency for vapor degreasing, or low dielectric constant if the application involves exposure or potential exposure to electronic equipment or components). A need also continues to exist for improved fluids to transfer heat and/or mange the temperature of devices and articles, including in portable and hand-held electronic devices where the desire to miniaturize while adding functionality increases the thermal power density of the device while in operation, thus making cooling of the electronics components within such devices, including batteries, more challenging. As general rule increases in computational power within desktop computers, data centres, telecommunication centres and the like results in an increase in the heat output when such devices are operating, again making thermal management of such electronic devices increasingly important and increasingly more difficult and demanding. Other examples of thermal management challenges occur as a result of the increasing use of electronic vehicles, including particularly, cars, trucks, motorcycles and the like. In electric vehicles the thermal management function is especially important and challenging for several reasons, including the criticality of cooling and/or heating the batteries to be within a relatively narrow temperature range and in a way that is reliable, efficient and safe, and the challenge to provide effective thermal battery management is becoming greater as the demand for battery-operated vehicles with greater range and faster charging increases.

The efficiency and effectiveness of batteries, especially the batteries that provide the power in electronic vehicles, is a function of the operating temperature at which they operate. Thus, thermal management system must frequently be able to do more than simply remove heat from the battery during operation and/or charging—it must be able to effect cooling in a relatively narrow temperature range using equipment that is as low cost as possible and as light weight as possible. This results in the need for a heat transfer fluid in such systems that possesses a difficult-to-achieve combination of physical and performance properties. Furthermore, in some important applications the thermal management system must be able to add heat to the battery, especially as the vehicle is started in cold weather, which adds further to the difficulty of discovering and developing/obtaining compounds and/or compositions effective in such systems, not only from a thermal performance standpoint, but also a myriad of other standpoints, including environmental, safety (flammability and toxicity), dielectric properties, and others.

As a particular example of the importance of dielectric constant, one frequently used system for the thermal management of electric vehicle batteries involves immersing the battery in the fluid used for thermal management. Such systems add the additional constraint that the fluid used in such systems must be electronically compatible with the intimate contact with the battery, or other electronic device or component, while the battery or device is in operation. In general, this means the fluid must not only be non-flammable, but it must also have a low electrical conductivity and a high level of stability while in contact with the battery or other electronic component(s) while the component(s) are operating and at the relatively high temperatures existing during operation. Applicants have come to appreciate the desirability of such properties even in indirect cooling of operating electronic devices and batteries because leakage of any such fluid may result in contact with operating electronic components.

Perfluorinated compounds have heretofore frequently been used in many of these demanding applications. For example, the thermal management fluid which has been commonly used for battery cooling, including immersive cooling, is a water/glycol combination, although other classes of materials, including some chlorofluorocarbons, fluorohydrocarbons, chlorohydrocarbons and hydrofluoroethers, have been mentioned for possible use. See, for example, US 2018/0191038.

Fluorinated ether compounds according to the formula

(F₃C)₂CH—O—CHₙF₂₋ₙ—CHₘF₃₋ₘ (1)

where n is 1 or 2, and where m is any integer of 0 to 3 when n is 1, but when n is 2, then m is 0 or 2 have been suggested for use as solvents, particularly for various fluorine-containing polyethers. See JP202105950. This document indicates that an embodiment of Formula 1 that is said to have a 3-1 configuration (understood to mean that m=3 and n=1) have additional uses, including as a draining agent, blowing agent, heat transfer media and fire extinguishing agent, although no such uses are specifically described or exemplified.

The energy density of lithium-ion batteries can be substantially improved by carbon-based electrode materials with high capacity active materials, such as silicon. Yet, high-capacity materials present a new set of challenges not previously encountered with carbon-based materials. For example, the cycle life of cells built with high-capacity active materials and conventional electrolytes tends to be much shorter than the cycle life of cells built with carbon based active materials and the same electrolytes. The selection of electrolytes may impact formation of solid electrolyte interphase (SEI) layers, ionic mobility, and various other factors that collectively impact the cycle life of a cell. Specific electrolyte formulation may be necessary to address these new challenges presented by introducing high-capacity active materials into lithium ion batteries, and preferably these new electrolytes are also environmentally friendly and possess many of the other beneficial properties mentioned in connection with the heat transfer compositions.

Vapor phase soldering is another example of a process that utilizes heat transfer fluids. In this application, high temperatures are used and accordingly the heat transfer fluid must be suitable for high temperature exposure (e.g., up to 250° C.) Currently, perfluoropolyethers (PFPE, that is, compounds that have only carbon, oxygen and fluorine) are commonly used as the heat transfer fluids in this application. Although many PFPEs have adequate thermal stability for these high temperatures, they are environmentally persistent with extremely long atmospheric lifetimes which, in turn, gives rise to high global warming potentials (GWPs).

Thus, applicants have come to appreciate the need, among the other needs described herein, for thermal management methods and systems which use a heat transfer fluid which is environmentally acceptable (low GWP and low ODP), non-flammable, has low or no toxicity, has excellent insulating properties and has thermal properties that provide effective cooling and/or heat, including at relatively high temperatures and/or for use in operating electronic components in a relatively narrow temperature range with equipment that is preferably low cost, reliable and light weight, among other uses. for example, applicants have found that fluids that have relatively low boiling points (e.g., below 50° C.) are not desirable in many applications since the use of such fluids will tend to increase the cost and/or weight of the cooling equipment for many batteries and/or electronic cooling applications, and may also decrease reliability, as explained hereinafter.

### SUMMARY

The present invention includes novel compounds according to the following Formula I:

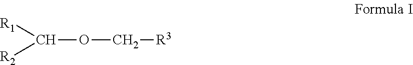

where
R¹, R² and R³ are each independently CₓR′_{(2x+1)-y}H_{y};
each R′ is independently selected from F or Cl and wherein the value of (2x+1)−y is the total number of R′ substituents on the indicated carbon atom(s);
each x is independently equal to or greater than 1 and equal to or less than 6; and
y is equal to or greater than 0 and less than or equal to 2x+1, provided: (i) that when each of R1 and R2 are CF₃ then R³ is neither CF₃ nor CH₂F; (ii) that the total number of F present in the compound is from 7 to 15; (iii) that (a) if the total number of R′ on the molecule is 8 or greater, then the ratio of R′ to H on the O—CH₂—R³ moiety is 1.5 or greater and (b) that when the total number of R′ on the molecule is 13 or greater, the ratio of R′ to H on the O—CH₂—R³ moiety is 2 or greater and (iv) that the compound has zero or 1 Cl substituents. For the purposes of convenience, any compound according to this paragraph is sometimes referred to herein as Compound 1.

The present invention includes novel compounds according to Compound 1 further provided that when the total number of R′ on the molecule is 13 or greater, the ratio of R′ to H on the O—CH2-R³ moiety is 2.5 or greater. For the purposes of convenience, any compound according to this paragraph is sometimes referred to herein as Compound 1^{A}.

The present invention includes novel compounds according to Compound 1 further provided that when the total number of R′ on the molecule is 13 or greater, the ratio of R′ to H on the O—CH2-R3 moiety is 3 or greater. For the purposes of convenience, any compound according to this paragraph is sometimes referred to herein as Compound 1^{B}.

The present invention includes novel compounds according to Compound 1 further provided that when the total number of R′ on the molecule is 13 or greater, the ratio of R′ to H on the O—CH2-R3 moiety is 3.5 or greater. For the purposes of convenience, any compound according to this paragraph is sometimes referred to herein as Compound 1^{C}.

The present invention includes novel compounds according to Compound 1 further provided that for each of R¹ and R² x is 1. For the purposes of convenience, any compound according to this paragraph is sometimes referred to herein as Compound 1^{D}.

The present also invention includes certain compositions comprising a compound represented by the following Formula Ia:

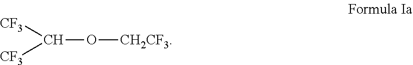

For the purposes of convenience, a compound according to this paragraph is sometimes referred to herein as Compound 1A. Compound 1A may also be named as propane, 2-(2′,2′,2′-trifluorethoxy)-(1,1,1,3,3,3-hexafluoro) or propane, 1,1,1,3,3,3-hexafluoro-2-(2,2,2,-trifluorethoxy)-. Applicants have found that this compound has surprising and unexpected advantages when used in several applications, including particularly in heat transfer applications (especially cooling of electronic devices, equipment and batteries, including of immersion cooling of same) and in solvent applications. These unexpected advantages occur in part because of applicant determining that the use of this compound permits the use of fluids in such applications have at the same time low GWP (below 200), low dielectric constant (e.g., below 4), no flash point and an advantageous normal boiling point of about 69° C.

The present invention includes a novel compound represented by the following Formula Ib:

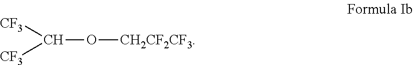

For the purposes of convenience, a compound according to this paragraph is sometimes referred to herein as Compound 1B.

The present invention includes a novel compound represented by the following Formula Ic:

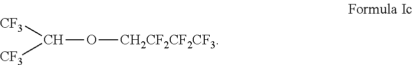

For the purposes of convenience, a compound according to this paragraph is sometimes referred to herein as Compound 1C.

The present invention includes a novel compound represented by the following Formula Id:

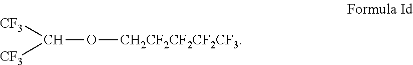

For the purposes of convenience, a compound according to this paragraph is sometimes referred to herein as Compound 1D. The present invention includes a novel compound represented by the following Formula Ie:

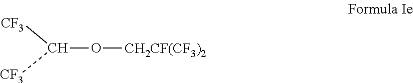

For the purposes of convenience, a compound according to this paragraph is sometimes referred to herein as Compound 1E.

The present invention includes a novel compound represented by the following Formula If:

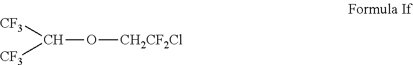

For the purposes of convenience, a compound according to this paragraph is sometimes referred to herein as Compound 1F.

The present invention includes novel compounds according to the following Formula I:

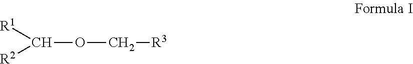

where
R¹, R² and R³ are each independently CₓR′_{(2x+1)-y}H_{y};
each R′ is independently selected from F or Cl and wherein the value of (2x+1)−y is the total number of R′ substituents on the indicated carbon atom(s);
each x is independently equal to or greater than 1 and equal to or less than 6; and

y is equal to or greater than 0 and less than or equal to 2x+1, provided: (i) that when each of R1 and R2 are CF₃ then R³ is neither CF₃ nor CH₂F; (ii) that the total number of F present in the compound is from 7 to 15; (iii) that (a) if the total number of R′ on the molecule is 8 or greater, then the ratio of R′ to H on the O—CH₂—R³ moiety is 1.5 or greater and (b) that when the total number of R′ on the molecule is 13 or greater, the ratio of R′ to H on the O—CH₂—R³ moiety is 2 or greater; (iii) that the compound has from zero or one Cl substituents; and (iv) R³ includes at least one CF₃ and X is 2 or greater. For the purposes of convenience, any compound according to this paragraph is sometimes referred to herein as Compound 2.

The present invention includes novel compounds according to Compound 2 further provided that when the total number of R′ on the molecule is 13 or greater, the ratio of R′ to H on the O—CH2-R³ moiety is 2.5 or greater. For the purposes of convenience, any compound according to this paragraph is sometimes referred to herein as Compound 2A.

The present invention includes novel compounds according to Compound 2 further provided that when the total number of R′ on the molecule is 13 or greater, the ratio of R′ to H on the O—CH2-R3 moiety is 3 or greater. For the purposes of convenience, any compound according to this paragraph is sometimes referred to herein as Compound 2B.

The present invention includes novel compounds according to Compound 2 further provided that when the total number of R′ on the molecule is 13 or greater, the ratio of R′ to H on the O—CH2-R3 moiety is 3.5 or greater. For the purposes of convenience, any compound according to this paragraph is sometimes referred to herein as Compound 2C.

The present invention includes novel compounds according to Compound 2 further provided that for each of R₁ and R₂ x is 1. For the purposes of convenience, any compound according to this paragraph is sometimes referred to herein as Compound 2D.

The present invention includes novel compounds according to the following Formula I:

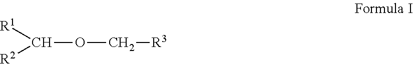

where R¹, R² and R³ are each independently CₓF_{(2x+1)-y}H_{y};

each x is independently equal to or greater than 1 and equal to or less than 6; and

y is equal to or greater than 0 and less than or equal to 2x+1, provided: (i) that when each of R¹ and R² are CF₃ then R³ is neither CF₃ nor CH₂F; (ii) that the total number of F present in the compound is from 7 to 15; and (iii) that (a) if the total number of F on the molecule is 8 or greater, then the ratio of R′ to H on the O—CH₂—R³ moiety is 1.5 or greater and (b) that when the total number of F on the molecule is 13 or greater, the ratio of F to H on the O—CH₂—R³ moiety is 2 or greater. For the purposes of convenience, any compound according to this paragraph is sometimes referred to herein as Compound 3.

The present invention includes novel compounds according to Compound 3 further provided that when the total number of F on the molecule is 13 or greater, the ratio of F to H on the O—CH₂—R³ moiety is 2.5 or greater. For the purposes of convenience, any compound according to this paragraph is sometimes referred to herein as Compound 3A.

The present invention includes novel compounds according to Compound 3 further provided that when the total number of F on the molecule is 13 or greater, the ratio of F to H on the O—CH2-R3 moiety is 3 or greater. For the purposes of convenience, any compound according to this paragraph is sometimes referred to herein as Compound 3B.

The present invention includes novel compounds according to Compound 4 wherein when the total number of F on the molecule is 13 or greater, the ratio of F to H on the O—CH2-R3 moiety is 3.5 or greater. For the purposes of convenience, any compound according to this paragraph is sometimes referred to herein as Compound 3C.

The present invention includes novel compounds according to Compound 3 further provided that for each of R¹ and R² x is 1. For the purposes of convenience, any compound according to this paragraph is sometimes referred to herein as Compound 3D.

The present invention includes novel compounds according to the following Formula I:

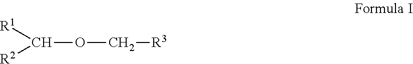

where R¹, R² and R³ are each independently CₓF_{(2x+1)-y}H_{y};

each x is independently equal to or greater than 1 and equal to or less than 6; and

y is equal to or greater than 0 and less than or equal to 2x+1, provided: (i) that when each of R¹ and R² are CF₃ then R³ is neither CF₃ nor CH₂F; (ii) that the total number of F present in the compound is from 7 to 15; (iii) that (a) if the total number of F on the molecule is 8 or greater, then the ratio of F to H on the O—CH₂—R³ moiety is 1.5 or greater and (b) that when the total number of F on the molecule is 13 or greater, the ratio of F to H on the O—CH₂—R³ moiety is 2 or greater; and (iv) R³ includes at least one CF₃ and X is 2 or greater. For the purposes of convenience, any compound according to this paragraph is sometimes referred to herein as Compound 4.

The present invention includes novel compounds according to Compound 3 further provided that when the total number of F on the molecule is 13 or greater, the ratio of F to H on the O—CH₂—R³ moiety is 2.5 or greater. For the purposes of convenience, any compound according to this paragraph is sometimes referred to herein as Compound 4A.

The present invention includes novel compounds according to Compound 3 further provided that when the total number of F on the molecule is 13 or greater, the ratio of F to H on the O—CH2-R3 moiety is 3 or greater. For the purposes of convenience, any compound according to this paragraph is sometimes referred to herein as Compound 4B.

The present invention includes novel compounds according to Compound 4 wherein when the total number of F on the molecule is 13 or greater, the ratio of F to H on the O—CH2-R3 moiety is 3.5 or greater. For the purposes of convenience, any compound according to this paragraph is sometimes referred to herein as Compound 4C.

The present invention includes novel compounds according to Compound 4 further provided that for each of R¹ and R² x is 1. For the purposes of convenience, any compound according to this paragraph is sometimes referred to herein as Compound 4D.

The present invention includes novel compounds according to the following Formula I:

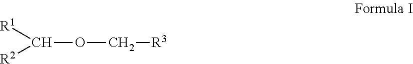

where R¹, R² and R³ are each independently CₓR′_{(2x+1)-y}H_{y};

each R′ is independently selected from F or Cl and wherein the value of (2x+1)−y is the total number of R′ substituents on the indicated carbon atom(s);

each x is independently equal to or greater than 1 and equal to or less than 6;

and

y is equal to or greater than 0 and less than or equal to 2x+1, provided: (i) that when each of R¹ and R² are CF₃ then R³ is neither CF₃ nor CH₂F; (ii) that the total number of F present in the compound is from 7 to 15; and (ii) that the following compounds are not included: (a) Propane, 2-(2,2-difluoroethoxy)-1,1,1,3,3,3-hexafluoro; (b) Propane, 1,1,1,3,3,3-hexafluoro-2-(2,2,2-trifluoroethoxy)-; (c) Propane, 1,1,1,3,3,3-hexafluoro-2-(2,2,3,3-tetrafluoropropoxy)-; (d) Pentane, 1,1,1,2,2,4,4,5,5,5-decafluoro-3-(2,2,2-trifluoroethoxy)-; (e) Pentane, 1,1,2,2,3,3,4,4-octafluoro-5-[2,2,2-trifluoro-1-(trifluoromethyl)ethoxy]; and (f) Hexane, 1,1,1,2,2,3,3,5,5,6,6,6-dodecafluoro-4-(2,2,2-trifluoroethoxy); and (iii) that the compound has zero or 1 Cl substituents. For the purposes of convenience, any compound according to this paragraph is sometimes referred to herein as Compound 5.

Useful in compositions, systems and methods of the present invention are compounds according to the following Formula I:

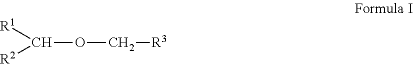

where
R¹, R² and R³ are each independently CₓR′_{(2x+1)-y}H_{y};
each R′ is independently selected from F or Cl and wherein the value of (2x+1)−y is the total number of R′ substituents on the indicated carbon atom(s);
each x is independently equal to or greater than 1 and equal to or less than 6; and
y is equal to or greater than 0 and less than or equal to 2x+1, provided: (i) that the total number of F present in the compound is from 7 to 15; (ii) that (a) if the total number of R′ on the molecule is 8 or greater, then the ratio of R′ to H on the O—CH₂—R³ moiety is 1.5 or greater and (b) that when the total number of R′ on the molecule is 13 or greater, the ratio of R′ to H on the O—CH₂—R³ moiety is 2 or greater and (iii) that the compound has zero or 1 Cl substituents. For the purposes of convenience, any compound according to this paragraph is sometimes referred to herein as Compound 6.

In preferred embodiments, the compositions of the present invention comprise one or more compounds of the present invention and have properties as specified in the following Table 1, with the composition number appearing in bold in the first column (abbreviated as “Comp. No.”) and being used hereinafter to reference a composition containing having the compound(s) and/or properties specified in the corresponding row (measured as defined herein), with the reference NR meaning that the indicated property is not required for the that composition:

TABLE 1

| | | Property | | | | | |
|---|---|---|---|---|---|---|---|
| Composition | | | | Dielectric | | Thermal | Boiling |
| No. | Compound(s) | GWP | Flammability | Constant | Toxicity | Conductivity | Point |
| 1 | 1 | NR | Non- | <4 | Ames- | NR | 25-150 |
| | | | flammable | | negative | | |
| 1A | 1A | NR | NR | NR | NR | NR | NR |
| 1A1 | 1A | <200 | Non- | NR | NR | NR | NR |
| | | | flammable | | | | |
| 1A2 | 1A | <200 | Non- | <4.5 | NR | NR | NR |
| | | | flammable | | | | |
| 1A3 | 1A | <200 | Non- | <4 | NR | NR | NR |
| | | | flammable | | | | |
| 1A4 | 1A | <200 | Non- | <3.5 | NR | NR | NR |
| | | | flammable | | | | |
| 1A5 | 1A | <200 | Non- | <3 | NR | NR | NR |
| | | | flammable | | | | |
| 1A6 | 1A | <200 | Non- | <4 | Ames- | >0.055 | 25-150 |
| | | | flammable | | negative | | |
| 1A7 | 1A | <200 | Non- | <3.5 | Ames- | NR | 50-150 |
| | | | flammable | | negative | | |
| 1A8 | 1A | <200 | Non- | <3 | Ames- | NR | 50-150 |
| | | | flammable | | negative | | |
| 1A9 | 1^{A} | <200 | Non- | <3 | Ames- | NR | 50-150 |
| | | | flammable | | negative | | |
| 1A10 | 1^{B} | <200 | Non- | <3 | Ames- | NR | 50-150 |
| | | | flammable | | negative | | |
| 1A11 | 1^{C} | <200 | Non- | <3 | Ames- | NR | 50-150 |
| | | | flammable | | negative | | |
| 1A12 | 1^{D} | <200 | Non- | <3 | Ames- | NR | 50-150 |
| | | | flammable | | negative | | |
| 1B | 1B | NR | NR | NR | NR | NR | NR |
| 1B1 | 1B | <200 | Non- | NR | NR | NR | NR |
| | | | flammable | | | | |
| 1B2 | 1B | <200 | Non- | <4.5 | NR | NR | NR |
| | | | flammable | | | | |
| 1B3 | 1B | <200 | Non- | <4 | NR | NR | NR |
| | | | flammable | | | | |
| 1B4 | 1B | <200 | Non- | <3.5 | NR | NR | NR |
| | | | flammable | | | | |
| 1B5 | 1B | <200 | Non- | <3 | NR | NR | NR |
| | | | flammable | | | | |
| 1B6 | 1B | <200 | Non- | <4 | Ames- | >0.055 | 25-150 |
| | | | flammable | | negative | | |
| 1B7 | 1B | <200 | Non- | <3.5 | Ames- | NR | 50-150 |
| | | | flammable | | negative | | |
| 1B8 | 1B | <200 | Non- | <3 | Ames- | NR | 50-150 |
| | | | flammable | | negative | | |
| 1C | 1C | NR | NR | NR | NR | NR | NR |
| 1C1 | 1C | <200 | Non- | NR | NR | NR | NR |
| | | | flammable | | | | |
| 1C2 | 1C | <200 | Non- | <4.5 | NR | NR | NR |
| | | | flammable | | | | |
| 1C3 | 1C | <200 | Non- | <4 | NR | NR | NR |
| | | | flammable | | | | |
| 1C4 | 1C | <200 | Non- | <3.5 | NR | NR | NR |
| | | | flammable | | | | |
| 1C5 | 1C | <200 | Non- | <3 | NR | NR | NR |
| | | | flammable | | | | |
| 1C6 | 1C | <200 | Non- | <4 | Ames- | >0.055 | 25-150 |
| | | | flammable | | negative | | |
| 1C7 | 1C | <200 | Non- | <3.5 | Ames- | NR | 50-150 |
| | | | flammable | | negative | | |
| 1C8 | 1C | <200 | Non- | <3 | Ames- | NR | 50-150 |
| | | | flammable | | negative | | |
| 1D | 1D | NR | NR | NR | NR | NR | NR |
| 1D1 | 1D | <200 | Non- | NR | NR | NR | NR |
| | | | flammable | | | | |
| 1D2 | 1D | <200 | Non- | <4.5 | NR | NR | NR |
| | | | flammable | | | | |
| 1D3 | 1D | <200 | Non- | <4 | NR | NR | NR |
| | | | flammable | | | | |
| 1D4 | 1D | <200 | Non- | <3.5 | NR | NR | NR |
| | | | flammable | | | | |
| 1D5 | 1D | <200 | Non- | <3 | NR | NR | NR |
| | | | flammable | | | | |
| 1D6 | 1D | <200 | Non- | <4 | Ames- | >0.055 | 25-150 |
| | | | flammable | | negative | | |
| 1D7 | 1D | <200 | Non- | <3.5 | Ames- | NR | 50-150 |
| | | | flammable | | negative | | |
| 1D8 | 1D | <200 | Non- | <3 | Ames- | NR | 50-150 |
| | | | flammable | | negative | | |
| 1E | 1E | NR | NR | NR | NR | NR | NR |
| 1E1 | 1E | <200 | Non- | NR | NR | NR | NR |
| | | | flammable | | | | |
| 1E2 | 1E | <200 | Non- | <4.5 | NR | NR | NR |
| | | | flammable | | | | |
| 1E3 | 1E | <200 | Non- | <4 | NR | NR | NR |
| | | | flammable | | | | |
| 1E4 | 1E | <200 | Non- | <3.5 | NR | NR | NR |
| | | | flammable | | | | |
| 1E5 | 1E | <200 | Non- | <3 | NR | NR | NR |
| | | | flammable | | | | |
| 1E6 | 1E | <200 | Non- | <4 | Ames- | >0.055 | 25-150 |
| | | | flammable | | negative | | |
| 1E7 | 1E | <200 | Non- | <3.5 | Ames- | NR | 50-150 |
| | | | flammable | | negative | | |
| 1E8 | 1E | <200 | Non- | <3 | Ames- | NR | 50-150 |
| | | | flammable | | negative | | |
| 1F | 1F | NR | NR | NR | NR | NR | NR |
| 1F1 | 1F | <200 | Non- | NR | NR | NR | NR |
| | | | flammable | | | | |
| 1F2 | 1F | <200 | Non- | <4.5 | NR | NR | NR |
| | | | flammable | | | | |
| 1F3 | 1F | <200 | Non- | <4 | NR | NR | NR |
| | | | flammable | | | | |
| 1F4 | 1F | <200 | Non- | <3.5 | NR | NR | NR |
| | | | flammable | | | | |
| 1F5 | 1F | <200 | Non- | <3 | NR | NR | NR |
| | | | flammable | | | | |
| 1F6 | 1F | <200 | Non- | <4 | Ames- | >0.055 | 25-150 |
| | | | flammable | | negative | | |
| 1F7 | 1F | <200 | Non- | <3.5 | Ames- | NR | 50-150 |
| | | | flammable | | negative | | |
| 1F8 | 1F | <200 | Non- | <3 | Ames- | NR | 50-150 |
| | | | flammable | | negative | | |
| 2 | 2 | NR | NR | NR | NR | NR | NR |
| 2A1 | 2 | <200 | Non- | NR | NR | NR | NR |
| | | | flammable | | | | |
| 2A2 | 2 | <200 | Non- | <4.5 | NR | NR | NR |
| | | | flammable | | | | |
| 2A3 | 2 | <200 | Non- | <4 | NR | NR | NR |
| | | | flammable | | | | |
| 2A4 | 2 | <200 | Non- | <3.5 | NR | NR | NR |
| | | | flammable | | | | |
| 2A5 | 2 | <200 | Non- | <3 | NR | NR | NR |
| | | | flammable | | | | |
| 2A6 | 2 | <200 | Non- | <4 | Ames- | >0.055 | 25-150 |
| | | | flammable | | negative | | |
| 2A7 | 2 | <200 | Non- | <3.5 | Ames- | NR | 50-150 |
| | | | flammable | | negative | | |
| 2A8 | 2 | <200 | Non- | <3 | Ames- | NR | 50-150 |
| | | | flammable | | negative | | |
| 3 | 3 | NR | NR | NR | NR | NR | NR |
| 3A1 | 3 | <200 | Non- | NR | NR | NR | NR |
| | | | flammable | | | | |
| 3A2 | 3 | <200 | Non- | <4.5 | NR | NR | NR |
| | | | flammable | | | | |
| 3A3 | 3 | <200 | Non- | <4 | NR | NR | NR |
| | | | flammable | | | | |
| 3A4 | 3 | <200 | Non- | <3.5 | NR | NR | NR |
| | | | flammable | | | | |
| 3A5 | 3 | <200 | Non- | <3 | NR | NR | NR |
| | | | flammable | | | | |
| 3A6 | 3 | <200 | Non- | <4 | Ames- | >0.055 | 25-150 |
| | | | flammable | | negative | | |
| 3A7 | 3 | <200 | Non- | <3.5 | Ames- | NR | 50-150 |
| | | | flammable | | negative | | |
| 3A8 | 3 | <200 | Non- | <3 | Ames- | NR | 50-150 |
| | | | flammable | | negative | | |
| 4 | 4 | NR | NR | NR | NR | NR | NR |
| 4A1 | 4 | <200 | Non- | NR | NR | NR | NR |
| | | | flammable | | | | |
| 4A2 | 4 | <200 | Non- | <4.5 | NR | NR | NR |
| | | | flammable | | | | |
| 4A3 | 4 | <200 | Non- | <4 | NR | NR | NR |
| | | | flammable | | | | |
| 4A4 | 4 | <200 | Non- | <3.5 | NR | NR | NR |
| | | | flammable | | | | |
| 4A5 | 4 | <200 | Non- | <3 | NR | NR | NR |
| | | | flammable | | | | |
| 4A6 | 4 | <200 | Non- | <4 | Ames- | >0.055 | 25-150 |
| | | | flammable | | negative | | |
| 4A7 | 4 | <200 | Non- | <3.5 | Ames- | NR | 50-150 |
| | | | flammable | | negative | | |
| 4A8 | 4 | <200 | Non- | <3 | Ames- | NR | 50-150 |
| | | | flammable | | negative | | |
| 5 | 5 | NR | NR | NR | NR | NR | NR |
| 5A1 | 5 | <200 | Non- | NR | NR | NR | NR |
| | | | flammable | | | | |
| 5A2 | 5 | <200 | Non- | <4.5 | NR | NR | NR |
| | | | flammable | | | | |
| 5A3 | 5 | <200 | Non- | <4 | NR | NR | NR |
| | | | flammable | | | | |
| 5A4 | 5 | <200 | Non- | <3.5 | NR | NR | NR |
| | | | flammable | | | | |
| 5A5 | 5 | <200 | Non- | <3 | NR | NR | NR |
| | | | flammable | | | | |
| 5A6 | 5 | <200 | Non- | <4 | Ames- | >0.055 | 25-150 |
| | | | flammable | | negative | | |
| 5A7 | 5 | <200 | Non- | <3.5 | Ames- | NR | 50-150 |
| | | | flammable | | negative | | |
| 5A8 | 5 | <200 | Non- | <3 | Ames- | NR | 50-150 |
| | | | flammable | | negative | | |
| 6 | 6 | NR | NR | NR | NR | NR | NR |
| 2A1 | 6 | <200 | Non- | NR | NR | NR | NR |
| | | | flammable | | | | |
| 6A2 | 6 | <200 | Non- | <4.5 | NR | NR | NR |
| | | | flammable | | | | |
| 6A3 | 6 | <200 | Non- | <4 | NR | NR | NR |
| | | | flammable | | | | |
| 6A4 | 6 | <200 | Non- | <3.5 | NR | NR | NR |
| | | | flammable | | | | |
| 6A5 | 6 | <200 | Non- | <3 | NR | NR | NR |
| | | | flammable | | | | |
| 6A6 | 6 | <200 | Non- | <4 | Ames- | >0.055 | 25-150 |
| | | | flammable | | negative | | |
| 6A7 | 6 | <200 | Non- | <3.5 | Ames- | NR | 50-150 |
| | | | flammable | | negative | | |
| 6A8 | 6 | <200 | Non- | <3 | Ames- | NR | 50-150 |
| | | | flammable | | negative | | |

The present invention provides a variety of uses for the composition of the present compounds, including each of Compounds 1-6 and the present compositions, including each of Compositions 1-6, and includes methods associated with such uses.

As used herein, reference to a group of compounds, compositions, methods, and the like, defined by numbers, such as the reference in the preceding paragraph to “any of Compounds 1-6” specifically includes all such numbered compounds, including any and all number compositions with a suffix. Thus, for example reference to “Compounds 1-6” includes each of the Compounds 1, including for example numbered compounds with a suffix such as a through f.

Thus, the present invention includes use of each of the present compounds, including each of Compounds 1-6, as a heat transfer fluid (including particularly immersion cooling), as a solvent (including vapor degreasing and other cleaning techniques, and as an etchant), as a carrier (including for coating), as an electrical insulator, as a blowing agent, as a flame suppressant, and as a flammability reducer, as explained in more detail hereinafter.

Thus, the invention includes methods for removing heat and/or energy from an article, device or fluid or adding heat and/or energy to an article, device or fluid comprising:

(a) providing the article, device or fluid; and

(b) transferring said heat and/or energy from and/or to any compound according to any of Compounds 1-6. For the purposes of convenience, methods according to this paragraph are sometimes referred to herein as Heat Transfer Methods 1.

The present invention includes methods for removing heat and/or energy from an article, device or fluid or adding heat and/or energy to an article, device or fluid comprising:

(a) providing the article, device or fluid; and

(b) transferring said heat and/or energy from and/or to any composition according to any of Compositions 1-16. For the purposes of convenience, methods according to this paragraph are sometimes referred to herein as Heat Transfer Methods 2.

The present invention includes methods for immersion cooling of an article or device:
(a) providing the article, device or fluid;
(b) providing a cooling fluid comprising a compound of the present invention, including each compound within Compounds 1-6; and
(c) removing heat from and/or adding heat to said article device by immersing at least a part or portion of said article or device in said fluid.
   For the purposes of convenience, methods according to this paragraph are sometimes referred to herein as Immersion Cooling Methods 1.

The present invention includes methods for immersion cooling of an article or device:
(d) providing the article, device or fluid;
(e) providing a cooling fluid comprising a composition of the present invention, including each composition within Compositions 1-6; and
(f) removing heat from and/or adding heat to said article device by immersing at least a part or portion of said article or device in said fluid.
   For the purposes of convenience, methods according to this paragraph are sometimes referred to herein as Immersion Cooling Methods 2.

The present invention includes methods for maintaining the temperature of an article, device or fluid within a temperature range by removing and/or adding heat to the article, device or fluid comprising:
(g) providing the article, device or fluid;
(h) providing a thermal management fluid comprising a compound of the present invention, including each compound within any of Compounds 1-6; and
(i) removing heat from and/or adding heat to said article device or fluid using said thermal management fluid.
   For the purposes of convenience, methods according to this paragraph are sometimes referred to herein as Thermal Management Methods 1.

The present invention includes methods for maintaining the temperature of an article, device or fluid within a temperature range by removing and/or adding heat to the article, device or fluid comprising:
(j) providing the article, device or fluid;
(k) providing a thermal management fluid comprising a composition of the present invention, including each composition within any of Compositions 1-6; and
(l) removing heat from and/or adding heat to said article device or fluid using said thermal management fluid.
   For the purposes of convenience, methods according to this paragraph are sometimes referred to herein as Thermal Management Methods 2.

The present invention includes systems and devices that include a heat transfer fluid for transferring heat within and/or to and/or from that system or device, said system and/or device comprising: (a) a system or device for transferring heat; and (b) in said system or device a heat transfer fluid comprising a compound according to any of Compounds 1-6. For the purposes of convenience, systems and/or devices according to this paragraph are sometimes referred to herein as Heat Transfer System 1.

The present invention includes systems and devices that include a heat transfer fluid for transferring heat within and/or to and/or from that system or device, said system and/or device comprising: (a) a system or device for transferring heat; and (b) in said system or device a heat transfer fluid comprising a composition according to any of Compositions 1-6. For the purposes of convenience, systems and/or devices according to this paragraph are sometimes referred to herein as Heat Transfer System 2.

The present invention includes methods for solvent cleaning an article or device or substrate, or a portion of an article or device or substrate comprising:

(a) providing the article, device or substrate; and

(b) contacting said article, device or substrate with a compound within any of Compounds 1-6. For the purposes of convenience, methods according to this paragraph are sometimes referred to herein as Cleaning Method 1.

The present invention includes methods for solvent cleaning an article or device or substrate, or a portion of an article or device or substrate comprising:

(a) providing the article, device or substrate; and

(b) contacting said article, device or substrate with a composition within any of Compositions 1-6. For the purposes of convenience, methods according to this paragraph are sometimes referred to herein as Cleaning Method 2.

The present invention includes methods for vapor degreasing an article or device or substrate, or a portion of an article or device or substrate comprising:

(a) providing the article, device or substrate; and

(b) vapor degreasing the article, device or substrate with a compound within any of Compounds 1-6. For the purposes of convenience, methods according to this paragraph are sometimes referred to herein as Vapor Degreasing Method 1.

The present invention includes methods for vapor degreasing an article or device or substrate, or a portion of an article or device or substrate comprising:

(a) providing the article, device or substrate; and

(b) vapor degreasing the article, device or substrate with a composition within any of Compositions 1-6. For the purposes of convenience, methods according to this paragraph are sometimes referred to herein as Vapor Degreasing Method 2.

The present invention includes methods for solvating a material comprising:

(a) providing a material to be solvated; and

(b) contacting said material with a compound within any of Compounds 1-5. For the purposes of convenience, methods according to this paragraph are sometimes referred to herein as Solvating Method 1.

The present invention includes methods for solvating a material comprising:

(a) providing a material to be solvated; and

(b) solvating the material in a composition within any of Compositions 1-6. For the purposes of convenience, methods according to this paragraph are sometimes referred to herein as Solvating Method 2.

The present invention includes methods for insulating an electronic or electrical article or device or substrate, or a portion of an article or device or substrate comprising:

(a) providing the article, device or substrate; and
(b) contacting said the article, device or substrate with a compound within any of Compounds 1-6. For the purposes of convenience, methods according to this paragraph are sometimes referred to herein as Electrical Insulating Method 1.

The present invention includes systems, devices and components that include an insulated electronic device or component comprising a composition within any of Compositions 1-6 as an insulator for said system, device or component. For the purposes of convenience, methods according to this paragraph are sometimes referred to herein as Insulated Electronic System 1.

The present invention includes methods for etching comprising:
(a) providing a substrate to be etched;
(b) providing a compound within any of Compounds 1-6; and
(c) introducing said compound to said substrate to be etched. For the purposes of convenience, methods according to this paragraph are sometimes referred to herein as Etching Method 1.

The present invention includes methods for etching comprising:
(d) providing a substrate to be etched;
(e) providing a composition within any of Compositions 1-6; and
(c) introducing said compound to said substrate to be etched. For the purposes of convenience, methods according to this paragraph are sometimes referred to herein as Etching Method 2.

The present invention includes methods for suppression of a flame comprising:

(a) providing a compound within any of Compounds 1-6; and
(b) introducing said compound into the flame and/or into the vicinity of the. For the purposes of convenience, methods according to this paragraph are sometimes referred to herein as Flame Suppression Method 1.

The present invention includes methods for suppression of a flame comprising:

(a) providing a composition according to any of Compositions 1-6; and
(b) introducing said composition into the flame and/or into the vicinity of the. For the purposes of convenience, methods according to this paragraph are sometimes referred to herein as Flame Suppression Method 2.

The present invention includes fire protection systems, that include a vessel storing a composition according to any of Compositions 1-6 and a conduit leading from said storage vessel to the site of a potential flame or fire. For the purposes of convenience, methods according to this paragraph are sometimes referred to herein as Fire Protection System 1.

The present invention includes methods of forming a thermosetting or thermoplastic or personal care foam comprising:

(a) providing a foamable composition comprising a foaming agent comprising a composition according to any of Compositions 1-6; and

(b) foaming said foamable composition. For the purposes of convenience, methods according to this paragraph are sometimes referred to herein as Foaming Method 1.

The present invention includes electrolyte formulations comprising:
(a) an electrolyte, preferably a lithium ion electrolyte;
(b) an organic solvent for the electrolyte; and
(c) any one or more compounds within any of Compounds 1-5, wherein said compound is either an organic solvent and/or an additive in the formulation.

For the purposes of convenience, electrolyte formulations according to this paragraph are sometimes referred to herein as Electrolyte Formulation 1.

The present invention includes electrolyte formulations comprising:
(c) an electrolyte, preferably a lithium ion electrolyte;
(d) an organic solvent for the electrolyte; and
(c) any one or more compositions within any of Compositions 1-6, wherein said composition is either an organic solvent and/or an additive in the formulation.

For the purposes of convenience, electrolyte formulations according to this paragraph are sometimes referred to herein as Electrolyte Formulation 2.

### BRIEF DESCRIPTION OF THE DRAWINGS

FIG. 1 is a schematic representation of a thermal management system of the present invention.

FIG. 2A is a schematic representation of a first exemplary immersion cooling system according to the present invention.

FIG. 2B is a schematic representation of a second exemplary immersion cooling system according to the present invention.

FIG. 3 is a schematic illustration of a battery thermal management system according to one embodiment of the present invention.

FIG. 4 is a photograph showing a battery thermal management system according to one embodiment of the present invention.

FIG. 5 is a schematic diagram of an exemplary organic Rankine cycle.

FIG. 6 is a schematic diagram of an exemplary heat pump.

FIG. 7 is a schematic diagram of an exemplary secondary loop system.

FIG. 8 is a semi-schematic drawing of one example of a lithium-ion battery cooling system using a composition of the present invention.

FIG. 9 is a semi-schematic drawing of one example of a lithium-ion battery having an electrolyte formulation of the present invention.

FIG. 10 is a semi-schematic drawing of a heat pipe using a heat transfer composition of the present invention.

FIG. 11 is a semi-schematic drawing of a vapor degreasing system using a heat transfer composition of the present invention.

### DETAILED DESCRIPTION

### Definitions

As used herein, the following terms have the meanings indicated below unless specifically indicated otherwise.

Electronic Device, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged, for example.

The term Heat Transfer Composition and related word forms, means a composition in the form of a fluid (liquid or gas) which is used to transfer heat or energy from one fluid, article or device to another fluid, article or device, and thus includes for example refrigerants, thermal management fluids and working fluids for Rankine cycles.

The term Rankine cycle as used herein refers to systems which include: 1) a boiler to change liquid to vapor at high pressure; 2) a turbine to expand the vapor to derive mechanical energy; 3) a condenser to change low pressure exhaust vapor from the turbine to low pressure liquid; and 4) a pump to move condensate liquid back to the boiler at high pressure. Such systems are commonly used for electrical power generation.

When a heat transfer composition is used in thermal management to keep a device or article within a particular temperature range (e.g., in electronic cooling), it is sometimes referred herein as a thermal management fluid.

The component(s) that are present in a heat transfer composition for the purpose of transferring heat (as opposed to, for example, providing lubrication or stabilization) in a heat transfer system (e.g., a vapour compression heat transfer system), that component or combination of components are sometimes referred to herein as a refrigerant.

Operating Electronic Device, and related word forms, means a device, or a component of a device, which is in the process of performing its intended function by receiving, and/or transmitting and/or producing electrical energy and/or electronic signals. Thus, the term “operating electronic device” as used herein includes, for example, a battery which is in the process of providing a source of electrical energy to another component and also a battery which is being charged or recharged.

Thermal contact, and related forms thereof, includes direct contact with the surface and indirect contact though another body or fluid which facilitates the flow of heat between the surface and the fluid.

Thermal conductivity refers to the breakdown voltage in kV as measured in accordance with ASTM D7896-19.

Global Warming Potential (“GWP”) was developed to allow comparisons of the global warming impact of different gases. It is a measure of how much energy the emission of one ton of a gas will absorb over a given period of time, relative to the emission of one ton of carbon dioxide. The larger GWP, the more that a given gas warms the Earth compared to CO2 over that time period. The time period usually used for GWP is 100 years. GWP provides a common measure, which allows analysts to add up emission estimates of different gases.

LC₅₀ is a measure of the acute toxicity of a compound. The acute inhalation toxicity of a compound can be assessed using the method described in the OECD Guideline for Testing of Chemicals No. 403 “Acute Inhalation Toxicity” (2009), Method B.2. (Inhalation) of Commission Regulation (EC) No. 440/2008.

The term AMES-negative refers to a compound or composition which returns a negative result when tested under the Ames test as specified in the Toxic Substances Control Act of the United States.

Flash Point refers the lowest temperature at which vapors of the liquid will keep burning after the ignition source is removed as determined in accordance with ASTM D3828-16a.

Non-flammable in the context of heat transfer compositions, including thermal management composition or fluid, means compounds or compositions which do not have a flash point below 100° F. (37.8° C.) in accordance with NFPA 30: Flammable and Combustible Liquid Code. The flash point of a thermal management composition or fluid refers the lowest temperature at which vapours of the composition will keep burning after the ignition source is removed as determined in accordance with ASTM D3828-16a.

In the context of a refrigerant composition, a compound or composition which is non-flammable and low or no-toxicity would be classified as “A1” by ASHRAE Standard 34-2016 Designation and Safety Classification of Refrigerants and described in Appendix B1 to ASHRAE Standard 34-2016.

No or low toxicity means a fluid classified as class “A” by ASHRAE Standard 34-2016 Designation and Safety Classification of Refrigerants and described in Appendix B1 to ASHRAE Standard 34-2016.

Capacity is the amount of cooling provided, in BTUs/hr, by the refrigerant in the refrigeration system. This is experimentally determined by multiplying the change in enthalpy in BTU/lb, of the refrigerant as it passes through the evaporator by the mass flow rate of the refrigerant. The enthalpy can be determined from the measurement of the pressure and temperature of the refrigerant. The capacity of the refrigeration system relates to the ability to maintain an area to be cooled at a specific temperature. The capacity of a refrigerant represents the amount of cooling or heating that it provides and provides some measure of the capability of a compressor to pump quantities of heat for a given volumetric flow rate of refrigerant. In other words, given a specific compressor, a refrigerant with a higher capacity will deliver more cooling or heating power.

Coefficient of Performance (hereinafter “COP”) is a universally accepted measure of refrigerant performance, especially useful in representing the relative thermodynamic efficiency of a refrigerant in a specific heating or cooling cycle involving evaporation or condensation of the refrigerant. In refrigeration engineering, this term expresses the ratio of useful refrigeration or cooling capacity to the energy applied by the compressor in compressing the vapor and therefore expresses the capability of a given compressor to pump quantities of heat for a given volumetric flow rate of a heat transfer fluid, such as a refrigerant. In other words, given a specific compressor, a refrigerant with a higher COP will deliver more cooling or heating power. One means for estimating COP of a refrigerant at specific operating conditions is from the thermodynamic properties of the refrigerant using standard refrigeration cycle analysis techniques (see for example, R. C. Downing, FLUOROCARBON REFRIGERANTS HANDBOOK, Chapter 3, Prentice-Hall, 1988 which is incorporated herein by reference in its entirety).

Vapor degreasing means a surface-cleaning process that uses solvent vapours to wash oils and other contaminants off of articles or parts of articles.

Dielectric Constant means the dielectric constant as measured in accordance with ASTM D150-11 at room temperature at 20 giga hertz (GHz).

Dielectric strength refers to the breakdown voltage in kV as measured in accordance with ASTM D87-13, Procedure A, with the modification that the spacing between the electrodes is 2.54 mm and the rate of rise was 500 V/sec.

As used herein, the singular forms “a”, “an”, and “the” include plural references unless the context clearly dictates otherwise.

As used herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

### Working Fluids

The present compounds and compositions are useful as working fluids for a variety of applications. As used herein, the term “working fluid” is used as a term which includes compositions of the present invention, which may include compounds or components other than those compounds of the present invention as described above. For convenience, such other components or compounds are referred to here-in as co-agents generally, which may be in a particular case a co-heat transfer agent, a co-solvent, a co-etchant, etc, as is specific to a particular application, method or system as discussed in detail hereinafter. The following Table 2 identifies working fluids that include compounds according to the present invention, including each of Compounds 1-6 and optionally a co-agent in the amounts as indicated based on the total weight of the components in the working fluid, with each amount being understood to be preceded by the word “about”:

TABLE 2
WORKING FLUIDS

| | Wt. % Compound of | Wt. % of |
|---|---|---|
| Working Fluid (WF) No. | the invention | Co-agent(s) |
| WF1-1 | 100 Compound 1 | 0 |
| WF1-2 | 1-99 Compound 1 | 1-99 |
| WF1-3 | 1-10 Compound 1 | 90-99 |
| WF1-4 | 30-50 Compound 1 | 50-70 |
| WF1-5 | 50-70 Compound 1 | 30-50 |
| WF1-6 | 70-90 Compound 1 | 10-30 |
| WF1-7 | 90-99 Compound 1 | 1-10 |
| WF1A1 | 100 Compound 1A | 0 |
| WF1A2 | 1-99 Compound 1A | 1-99 |
| WF1A3 | 1-10 Compound 1A | 90-99 |
| WF1A4 | 30-50 Compound 1A | 50-70 |
| WF1A5 | 50-70 Compound 1A | 30-50 |
| WF1A6 | 70-90 Compound 1A | 10-30 |
| WF1A7 | 90-99 Compound 1A | 1-10 |
| WF1B1 | 100 Compound 1B | 0 |
| WF1B2 | 1-99 Compound 1B | 1-99 |
| WF1B3 | 1-10 Compound 1B | 90-99 |
| WF1B4 | 30-50 Compound 1B | 50-70 |
| WF1B5 | 50-70 Compound 1B | 30-50 |
| WF1B6 | 70-90 Compound 1B | 10-30 |
| WF1B7 | 90-99 Compound 1B | 1-10 |
| WF1C1 | 100 Compound 1C | 0 |
| WF1C2 | 1-99 Compound 1C | 1-99 |
| WF1C3 | 1-10 Compound 1C | 90-99 |
| WF1C4 | 30-50 Compound 1C | 50-70 |
| WF1C5 | 50-70 Compound 1C | 30-50 |
| WF1C6 | 70-90 Compound 1C | 10-30 |
| WF1C7 | 90-99 Compound 1C | 1-10 |
| WF1D1 | 100 Compound 1D | 0 |
| WF1D2 | 1-99 Compound 1D | 1-99 |
| WF1D3 | 1-10 Compound 1D | 90-99 |
| WF1D4 | 30-50 Compound 1D | 50-70 |
| WF1D5 | 50-70 Compound 1D | 30-50 |
| WF1D6 | 70-90 Compound 1D | 10-30 |
| WF1D7 | 90-99 Compound 1D | 1-10 |
| WF1E1 | 100 Compound 1E | 0 |
| WF1E2 | 1-99 Compound 1E | 1-99 |
| WF1E3 | 1-10 Compound 1E | 90-99 |
| WF1E4 | 30-50 Compound 1E | 50-70 |
| WF1E5 | 50-70 Compound 1E | 30-50 |
| WF1E6 | 70-90 Compound 1E | 10-30 |
| WF1E7 | 90-99 Compound 1E | 1-10 |
| WF1F1 | 100 Compound 1F | 0 |
| WF1F2 | 1-99 Compound 1F | 1-99 |
| WF1F3 | 1-10 Compound 1F | 90-99 |
| WF1F4 | 30-50 Compound 1F | 50-70 |
| WF1F5 | 50-70 Compound 1F | 30-50 |
| WF1F6 | 70-90 Compound 1F | 10-30 |
| WF1F7 | 90-99 Compound 1F | 1-10 |
| WF2-1 | 100 Compound 2 | 0 |
| WF2-2 | 1-99 Compound 2 | 1-99 |
| WF2-3 | 1-10 Compound 2 | 90-99 |
| WF2-4 | 30-50 Compound 2 | 50-70 |
| WF2-5 | 50-70 Compound 2 | 30-50 |
| WF2-6 | 70-90 Compound 2 | 10-30 |
| WF2-7 | 90-99 Compound 2 | 1-10 |
| WF2A1 | 100 Compound 2A | 0 |
| WF2A2 | 1-99 Compound 2A | 1-99 |
| WF2A3 | 1-10 Compound 2A | 90-99 |
| WF2A4 | 30-50 Compound 2A | 50-70 |
| WF2A5 | 50-70 Compound 2A | 30-50 |
| WF2A6 | 70-90 Compound 2A | 10-30 |
| WF2A7 | 90-99 Compound 2A | 1-10 |
| WF2B1 | 100 Compound 2B | 0 |
| WF2B2 | 1-99 Compound 2B | 1-99 |
| WF2B3 | 1-10 Compound 2B | 90-99 |
| WF2B4 | 30-50 Compound 2B | 50-70 |
| WF2B5 | 50-70 Compound 2B | 30-50 |
| WF2B6 | 70-90 Compound 2B | 10-30 |
| WF2B7 | 90-99 Compound 2B | 1-10 |
| WF2C1 | 100 Compound 2C | 0 |
| WF2C2 | 1-99 Compound 2C | 1-99 |
| WF2C3 | 1-10 Compound 2C | 90-99 |
| WF2C4 | 30-50 Compound 2C | 50-70 |
| WF2C5 | 50-70 Compound 2C | 30-50 |
| WF2C6 | 70-90 Compound 2C | 10-30 |
| WF2C7 | 90-99 Compound 2C | 1-10 |
| WF2D1 | 100 Compound 2D | 0 |
| WF2D2 | 1-99 Compound 2D | 1-99 |
| WF2D3 | 1-10 Compound 2D | 90-99 |
| WF2D4 | 30-50 Compound 2D | 50-70 |
| WF2D5 | 50-70 Compound 2D | 30-50 |
| WF2D6 | 70-90 Compound 2D | 10-30 |
| WF2D7 | 90-99 Compound 2D | 1-10 |
| WF3-1 | 100 Compound 3 | 0 |
| WF3-2 | 1-99 Compound 3 | 1-99 |
| WF3-3 | 1-10 Compound 3 | 90-99 |
| WF3-4 | 30-50 Compound 3 | 50-70 |
| WF3-5 | 50-70 Compound 3 | 30-50 |
| WF3-6 | 70-90 Compound 3 | 10-30 |
| WF3-7 | 90-99 Compound 3 | 1-10 |
| WF3A1 | 100 Compound 3A | 0 |
| WF3A2 | 1-99 Compound 3A | 1-99 |
| WF3A3 | 1-10 Compound 3A | 90-99 |
| WF3A4 | 30-50 Compound 3A | 50-70 |
| WF3A5 | 50-70 Compound 3A | 30-50 |
| WF3A6 | 70-90 Compound 3A | 10-30 |
| WF3A7 | 90-99 Compound 3A | 1-10 |
| WF3B1 | 100 Compound 3B | 0 |
| WF3B2 | 1-99 Compound 3B | 1-99 |
| WF3B3 | 1-10 Compound 3B | 90-99 |
| WF3B4 | 30-50 Compound 3B | 50-70 |
| WF3B5 | 50-70 Compound 3B | 30-50 |
| WF3B6 | 70-90 Compound 3B | 10-30 |
| WF3B7 | 90-99 Compound 3B | 1-10 |
| WF3C1 | 100 Compound 3C | 0 |
| WF3C2 | 1-99 Compound 3C | 1-99 |
| WF3C3 | 1-10 Compound 3C | 90-99 |
| WF3C4 | 30-50 Compound 3C | 50-70 |
| WF3C5 | 50-70 Compound 3C | 30-50 |
| WF3C6 | 70-90 Compound 3C | 10-30 |
| WF3C7 | 90-99 Compound 3C | 1-10 |
| WF3D1 | 100 Compound 3D | 0 |
| WF3D2 | 1-99 Compound 3D | 1-99 |
| WF3D3 | 1-10 Compound 3D | 90-99 |
| WF3D4 | 30-50 Compound 3D | 50-70 |
| WF3D5 | 50-70 Compound 3D | 30-50 |
| WF3D6 | 70-90 Compound 3D | 10-30 |
| WF3D7 | 90-99 Compound 3D | 1-10 |
| WF4-1 | 100 Compound 4 | 0 |
| WF4-2 | 1-99 Compound 4 | 1-99 |
| WF4-3 | 1-10 Compound 4 | 90-99 |
| WF4-4 | 30-50 Compound 4 | 50-70 |
| WF4-5 | 50-70 Compound 4 | 30-50 |
| WF4-6 | 70-90 Compound 4 | 10-30 |
| WF4-7 | 90-99 Compound 4 | 1-10 |
| WF4A1 | 100 Compound 4A | 0 |
| WF4A2 | 1-99 Compound 4A | 1-99 |
| WF4A3 | 1-10 Compound 4A | 90-99 |
| WF4A4 | 30-50 Compound 4A | 50-70 |
| WF4A5 | 50-70 Compound 4A | 30-50 |
| WF4A6 | 70-90 Compound 4A | 10-30 |
| WF4A7 | 90-99 Compound 4A | 1-10 |
| WF4B1 | 100 Compound 4B | 0 |
| WF4B2 | 1-99 Compound 4B | 1-99 |
| WF4B3 | 1-10 Compound 4B | 90-99 |
| WF4B4 | 30-50 Compound 4B | 50-70 |
| WF4B5 | 50-70 Compound 4B | 30-50 |
| WF4B6 | 70-90 Compound 4B | 10-30 |
| WF4B7 | 90-99 Compound 4B | 1-10 |
| WF4C1 | 100 Compound 4C | 0 |
| WF4C2 | 1-99 Compound 4C | 1-99 |
| WF4C3 | 1-10 Compound 4C | 90-99 |
| WF4C4 | 30-50 Compound 4C | 50-70 |
| WF4C5 | 50-70 Compound 4C | 30-50 |
| WF4C6 | 70-90 Compound 4C | 10-30 |
| WF4C7 | 90-99 Compound 4C | 1-10 |
| WF4D1 | 100 Compound 4D | 0 |
| WF4D2 | 1-99 Compound 4D | 1-99 |
| WF4D3 | 1-10 Compound 4D | 90-99 |
| WF4D4 | 30-50 Compound 4D | 50-70 |
| WF4D5 | 50-70 Compound 4D | 30-50 |
| WF4D6 | 70-90 Compound 4D | 10-30 |
| WF4D7 | 90-99 Compound 4D | 1-10 |
| WF5-1 | 100 Compound 4 | 0 |
| WF5-2 | 1-99 Compound 5 | 1-99 |
| WF5-3 | 1-10 Compound 5 | 90-99 |
| WF5-4 | 30-50 Compound 5 | 50-70 |
| WF5-5 | 50-70 Compound 5 | 30-50 |
| WF5-6 | 70-90 Compound 5 | 10-30 |
| WF5-7 | 90-99 Compound 5 | 1-10 |
| WF6-1 | 100 Compound 4 | 0 |
| WF6-2 | 1-99 Compound 5 | 1-99 |
| WF6-3 | 1-10 Compound 5 | 90-99 |
| WF6-4 | 30-50 Compound 5 | 50-70 |
| WF6-5 | 50-70 Compound 5 | 30-50 |
| WF6-6 | 70-90 Compound 5 | 10-30 |
| WF6-7 | 90-99 Compound 5 | 1-10 |

### Heat Transfer Compositions

As mentioned above, the present invention provides various methods, processes and uses of the heat transfer compositions of the present invention, including each of Compositions 1-6 (i.e., liquids and/or gases) that may be used to transmit heat from one location to another (or from one body, or article or fluid to another bond, article or fluid). For example, the heat transfer compositions may be used to keep the temperature of a device below a defined upper and/or above a defined lower temperature. In another example, the heat transfer compositions may be used for energy conversion, as in the capture of waste heat from industrial or other processes and the conversion to electrical or mechanical energy.

The present invention this comprises the use of the working fluids of the present invention, including each of the working fluids defined by number in Table 1 above, as heat transfer composition of the present invention in which the co-agent, if present, is a co-heat transfer component. The following Table 3 identifies preferred heat transfer compositions of the present invention based on the working fluid definitions provided in Table 2 above, where the second column incorporates the compound identified for that WF No. and the amounts of the compound, and the co-heat transfer agent, if present, as if presented in the table below:

TABLE 3

| Heat | | | | | | |
|---|---|---|---|---|---|---|
| Transfer | | | | | | Boiling |
| Composition | WF Ref. | | | Dielectric | | Point, |
| (HTC) No. | No. | GWP | Flammability | Constant | Toxicity | ° C. |
| HTC1-1A1 | WF1-1 | <200 | Non- | <4 | Ames- | 25-150 |
| | | | flammable | | negative | |
| HTC1-1A2 | WF1-1 | <200 | Non- | <3 | Ames- | 50-150 |
| | | | flammable | | negative | |
| HTC1-2A1 | WF1-2 | <200 | Non- | <4 | Ames- | 25-150 |
| | | | flammable | | negative | |
| HTC1-2A2 | WF1-2 | <200 | Non- | <3 | Ames- | 50-150 |
| | | | flammable | | negative | |
| HTC1-3A1 | WF1-3 | <200 | Non- | <4 | Ames- | 25-150 |
| | | | flammable | | negative | |
| HTC1-3A2 | WF1-3 | <200 | Non- | <3 | Ames- | 50-150 |
| | | | flammable | | negative | |
| HTC1-4A1 | WF1-4 | <200 | Non- | <4 | Ames- | 25-150 |
| | | | flammable | | negative | |
| HTC1-4A2 | WF1-4 | <200 | Non- | <3 | Ames- | 50-150 |
| | | | flammable | | negative | |
| HTC1-5A1 | WF1-5 | <200 | Non- | <4 | Ames- | 25-150 |
| | | | flammable | | negative | |
| HTC1-5A2 | WF1-5 | <200 | Non- | <3 | Ames- | 50-150 |
| | | | flammable | | negative | |
| HTC1-6A1 | WF1-6 | <200 | Non- | <4 | Ames- | 25-150 |
| | | | flammable | | negative | |
| HTC1-6A2 | WF1-6 | <200 | Non- | <3 | Ames- | 50-150 |
| | | | flammable | | negative | |
| HTC1-7A1 | WF1-7 | <200 | Non- | <4 | Ames- | 25-150 |
| | | | flammable | | negative | |
| HTC1-7A2 | WF1-7 | <200 | Non- | <3 | Ames- | 50-150 |
| | | | flammable | | negative | |
| HTC1A1 | WF1A1 | <200 | Non- | <3 | Ames- | 50-150 |
| | | | flammable | | negative | |
| HTC1A2 | WF1A2 | <200 | Non- | <3 | Ames- | 50-150 |
| | | | flammable | | negative | |
| HTC1A3 | WF1A3 | <200 | Non- | <3 | Ames- | 50-150 |
| | | | flammable | | negative | |
| HTC1A4 | WF1A4 | <200 | Non- | <3 | Ames- | 50-150 |
| | | | flammable | | negative | |
| HTC1A5 | WF1A5 | <200 | Non- | <3 | Ames- | 50-150 |
| | | | flammable | | negative | |
| HTC1A6 | WF1A6 | <200 | Non- | <3 | Ames- | 50-150 |
| | | | flammable | | negative | |
| HTC1A7 | WF1A7 | <200 | Non- | <3 | Ames- | 50-150 |
| | | | flammable | | negative | |
| HTC1B1 | WF1B1 | <200 | Non- | <3 | Ames- | 50-150 |
| | | | flammable | | negative | |
| HTC1B2 | WF1B2 | <200 | Non- | <3 | Ames- | 50-150 |
| | | | flammable | | negative | |
| HTC1B3 | WF1B3 | <200 | Non- | <3 | Ames- | 50-150 |
| | | | flammable | | negative | |
| HTC1B4 | WF1B4 | <200 | Non- | <3 | Ames- | 50-150 |
| | | | flammable | | negative | |
| HTC1B5 | WF1B5 | <200 | Non- | <3 | Ames- | 50-150 |
| | | | flammable | | negative | |
| HTC1B6 | WF1B6 | <200 | Non- | <3 | Ames- | 50-150 |
| | | | flammable | | negative | |
| HTC1B7 | WF1B7 | <200 | Non- | <3 | Ames- | 50-150 |
| | | | flammable | | negative | |
| HTC1C1 | WF1C1 | <200 | Non- | <3 | Ames- | 50-150 |
| | | | flammable | | negative | |
| HTC1C2 | WF1C2 | <200 | Non- | <3 | Ames- | 50-150 |
| | | | flammable | | negative | |
| HTC1C3 | WF1C3 | <200 | Non- | <3 | Ames- | 50-150 |
| | | | flammable | | negative | |
| HTC1C4 | WF1C4 | <200 | Non- | <3 | Ames- | 50-150 |
| | | | flammable | | negative | |
| HTC1C5 | WF1C5 | <200 | Non- | <3 | Ames- | 50-150 |
| | | | flammable | | negative | |
| HTC1C6 | WF1C6 | <200 | Non- | <3 | Ames- | 50-150 |
| | | | flammable | | negative | |
| HTC1C7 | WF1C7 | <200 | Non- | <3 | Ames- | 50-150 |
| | | | flammable | | negative | |
| HTC1D1 | WF1D1 | <200 | Non- | <3 | Ames- | 50-150 |
| | | | flammable | | negative | |
| HTC1D2 | WF1D2 | <200 | Non- | <3 | Ames- | 50-150 |
| | | | flammable | | negative | |
| HTC1D3 | WF1D3 | <200 | Non- | <3 | Ames- | 50-150 |
| | | | flammable | | negative | |
| HTC1D4 | WF1D4 | <200 | Non- | <3 | Ames- | 50-150 |
| | | | flammable | | negative | |
| HTC1D5 | WF1D5 | <200 | Non- | <3 | Ames- | 50-150 |
| | | | flammable | | negative | |
| HTC1D6 | WF1D6 | <200 | Non- | <3 | Ames- | 50-150 |
| | | | flammable | | negative | |
| HTC1D7 | WF1D7 | <200 | Non- | <3 | Ames- | 50-150 |
| | | | flammable | | negative | |
| HTC1E1 | WF1E1 | <200 | Non- | <3 | Ames- | 50-150 |
| | | | flammable | | negative | |
| HTC1E2 | WF1E2 | <200 | Non- | <3 | Ames- | 50-150 |
| | | | flammable | | negative | |
| HTC1E3 | WF1E3 | <200 | Non- | <3 | Ames- | 50-150 |
| | | | flammable | | negative | |
| HTC1E4 | WF1E4 | <200 | Non- | <3 | Ames- | 50-150 |
| | | | flammable | | negative | |
| HTC1E5 | WF1E5 | <200 | Non- | <3 | Ames- | 50-150 |
| | | | flammable | | negative | |
| HTC1E6 | WF1E6 | <200 | Non- | <3 | Ames- | 50-150 |
| | | | flammable | | negative | |
| HTC1E7 | WF1E7 | <200 | Non- | <3 | Ames- | 50-150 |
| | | | flammable | | negative | |
| HTC1F1 | WF1F1 | <200 | Non- | <3 | Ames- | 50-150 |
| | | | flammable | | negative | |
| HTC1F2 | WF1F2 | <200 | Non- | <3 | Ames- | 50-150 |
| | | | flammable | | negative | |
| HTC1F3 | WF1F3 | <200 | Non- | <3 | Ames- | 50-150 |
| | | | flammable | | negative | |
| HTC1F4 | WF1F4 | <200 | Non- | <3 | Ames- | 50-150 |
| | | | flammable | | negative | |
| HTC1F5 | WF1F5 | <200 | Non- | <3 | Ames- | 50-150 |
| | | | flammable | | negative | |
| HTC1F6 | WF1F6 | <200 | Non- | <3 | Ames- | 50-150 |
| | | | flammable | | negative | |
| HTC1F7 | WF1F7 | <200 | Non- | <3 | Ames- | 50-150 |
| | | | flammable | | negative | |
| HTC2-1 | WF2-1 | <200 | Non- | <3 | Ames- | 50-150 |
| | | | flammable | | negative | |
| HTC2-2 | WF2-2 | <200 | Non- | <3 | Ames- | 50-150 |
| | | | flammable | | negative | |
| HTC2-3 | WFC2-3 | <200 | Non- | <3 | Ames- | 50-150 |
| | | | flammable | | negative | |
| HTC2-4 | WF2-4 | <200 | Non- | <3 | Ames- | 50-150 |
| | | | flammable | | negative | |
| HTC2-5 | WF2-5 | <200 | Non- | <3 | Ames- | 50-150 |
| | | | flammable | | negative | |
| HTC2-6 | WF2-6 | <200 | Non- | <3 | Ames- | 50-150 |
| | | | flammable | | negative | |
| HTC2-7 | WF2-7 | <200 | Non- | <3 | Ames- | 50-150 |
| | | | flammable | | negative | |
| HTC2A1 | WF2A1 | <200 | Non- | <3 | Ames- | 50-150 |
| | | | flammable | | negative | |
| HTC2A2 | WF2A2 | <200 | Non- | <3 | Ames- | 50-150 |
| | | | flammable | | negative | |
| HTC2A3 | WF2A3 | <200 | Non- | <3 | Ames- | 50-150 |
| | | | flammable | | negative | |
| HTC2A4 | WF2A4 | <200 | Non- | <3 | Ames- | 50-150 |
| | | | flammable | | negative | |
| HTC2A5 | WF2A5 | <200 | Non- | <3 | Ames- | 50-150 |
| | | | flammable | | negative | |
| HTC2A6 | WF2A6 | <200 | Non- | <3 | Ames- | 50-150 |
| | | | flammable | | negative | |
| HTC2A7 | WF2A7 | <200 | Non- | <3 | Ames- | 50-150 |
| | | | flammable | | negative | |
| HTC2B1 | WF2B1 | <200 | Non- | <3 | Ames- | 50-150 |
| | | | flammable | | negative | |
| HTC2B2 | WF2B2 | <200 | Non- | <3 | Ames- | 50-150 |
| | | | flammable | | negative | |
| HTC2B3 | WF2B3 | <200 | Non- | <3 | Ames- | 50-150 |
| | | | flammable | | negative | |
| HTC2B4 | WF2B4 | <200 | Non- | <3 | Ames- | 50-150 |
| | | | flammable | | negative | |
| HTC2B5 | WF2B5 | <200 | Non- | <3 | Ames- | 50-150 |
| | | | flammable | | negative | |
| HTC2B6 | WF2B6 | <200 | Non- | <3 | Ames- | 50-150 |
| | | | flammable | | negative | |
| HTC2B7 | WF2B7 | <200 | Non- | <3 | Ames- | 50-150 |
| | | | flammable | | negative | |
| HTC2C1 | WF2C1 | <200 | Non- | <3 | Ames- | 50-150 |
| | | | flammable | | negative | |
| HTC2C2 | WF2C2 | <200 | Non- | <3 | Ames- | 50-150 |
| | | | flammable | | negative | |
| HTC2C3 | WF2C3 | <200 | Non- | <3 | Ames- | 50-150 |
| | | | flammable | | negative | |
| HTC2C4 | WF2C4 | <200 | Non- | <3 | Ames- | 50-150 |
| | | | flammable | | negative | |
| HTC2C5 | WF2C5 | <200 | Non- | <3 | Ames- | 50-150 |
| | | | flammable | | negative | |
| HTC2C6 | WF2C6 | <200 | Non- | <3 | Ames- | 50-150 |
| | | | flammable | | negative | |
| HTC2C7 | WF2C7 | <200 | Non- | <3 | Ames- | 50-150 |
| | | | flammable | | negative | |
| HTC2D1 | WF2D1 | <200 | Non- | <3 | Ames- | 50-150 |
| | | | flammable | | negative | |
| HTC2D2 | WF2D2 | <200 | Non- | <3 | Ames- | 50-150 |
| | | | flammable | | negative | |
| HTC2D3 | WF2D3 | <200 | Non- | <3 | Ames- | 50-150 |
| | | | flammable | | negative | |
| HTC2D4 | WF2D4 | <200 | Non- | <3 | Ames- | 50-150 |
| | | | flammable | | negative | |
| HTC2D5 | WF2D5 | <200 | Non- | <3 | Ames- | 50-150 |
| | | | flammable | | negative | |
| HTC2D6 | WF2D6 | <200 | Non- | <3 | Ames- | 50-150 |
| | | | flammable | | negative | |
| HTC2D7 | WF2D7 | <200 | Non- | <3 | Ames- | 50-150 |
| | | | flammable | | negative | |
| HTC3-1 | WF3-1 | <200 | Non- | <3 | Ames- | 50-150 |
| | | | flammable | | negative | |
| HTC3-2 | WF3-2 | <200 | Non- | <3 | Ames- | 50-150 |
| | | | flammable | | negative | |
| HTC3-3 | WF3-3 | <200 | Non- | <3 | Ames- | 50-150 |
| | | | flammable | | negative | |
| HTC3-4 | WF3-4 | <200 | Non- | <3 | Ames- | 50-150 |
| | | | flammable | | negative | |
| HTC3-5 | WF3-5 | <200 | Non- | <3 | Ames- | 50-150 |
| | | | flammable | | negative | |
| HTC3-6 | WF3-6 | <200 | Non- | <3 | Ames- | 50-150 |
| | | | flammable | | negative | |
| HTC3-7 | WF3-7 | <200 | Non- | <3 | Ames- | 50-150 |
| | | | flammable | | negative | |
| HTC3A1 | WF3A1 | <200 | Non- | <3 | Ames- | 50-150 |
| | | | flammable | | negative | |
| HTC3A2 | WF3A2 | <200 | Non- | <3 | Ames- | 50-150 |
| | | | flammable | | negative | |
| HTC3A3 | WF3A3 | <200 | Non- | <3 | Ames- | 50-150 |
| | | | flammable | | negative | |
| HTC3A4 | WF3A4 | <200 | Non- | <3 | Ames- | 50-150 |
| | | | flammable | | negative | |
| HTC3A5 | WF3A5 | <200 | Non- | <3 | Ames- | 50-150 |
| | | | flammable | | negative | |
| HTC3A6 | WF3A6 | <200 | Non- | <3 | Ames- | 50-150 |
| | | | flammable | | negative | |
| HTC3A7 | WF3A7 | <200 | Non- | <3 | Ames- | 50-150 |
| | | | flammable | | negative | |
| HTC3B1 | WF3B1 | <200 | Non- | <3 | Ames- | 50-150 |
| | | | flammable | | negative | |
| HTC3B2 | WF3B2 | <200 | Non- | <3 | Ames- | 50-150 |
| | | | flammable | | negative | |
| HTC3B3 | WF3B3 | <200 | Non- | <3 | Ames- | 50-150 |
| | | | flammable | | negative | |
| HTC3B4 | WF3B4 | <200 | Non- | <3 | Ames- | 50-150 |
| | | | flammable | | negative | |
| HTC3B5 | WF3B5 | <200 | Non- | <3 | Ames- | 50-150 |
| | | | flammable | | negative | |
| HTC3B6 | WF3B6 | <200 | Non- | <3 | Ames- | 50-150 |
| | | | flammable | | negative | |
| HTC3B7 | WF3B7 | <200 | Non- | <3 | Ames- | 50-150 |
| | | | flammable | | negative | |
| HTC3C1 | WF3C1 | <200 | Non- | <3 | Ames- | 50-150 |
| | | | flammable | | negative | |
| HTC3C2 | WF3C2 | <200 | Non- | <3 | Ames- | 50-150 |
| | | | flammable | | negative | |
| HTC3C3 | WF3C3 | <200 | Non- | <3 | Ames- | 50-150 |
| | | | flammable | | negative | |
| HTC3C4 | WF3C4 | <200 | Non- | <3 | Ames- | 50-150 |
| | | | flammable | | negative | |
| HTC3C5 | WF3C5 | <200 | Non- | <3 | Ames- | 50-150 |
| | | | flammable | | negative | |
| HTC3C6 | WF3C6 | <200 | Non- | <3 | Ames- | 50-150 |
| | | | flammable | | negative | |
| HTC3C7 | WF3C7 | <200 | Non- | <3 | Ames- | 50-150 |
| | | | flammable | | negative | |
| HTC3D1 | WF3D1 | <200 | Non- | <3 | Ames- | 50-150 |
| | | | flammable | | negative | |
| HTC3D2 | WF3D2 | <200 | Non- | <3 | Ames- | 50-150 |
| | | | flammable | | negative | |
| HTC3D3 | WF3D3 | <200 | Non- | <3 | Ames- | 50-150 |
| | | | flammable | | negative | |
| HTC3D4 | WF3D4 | <200 | Non- | <3 | Ames- | 50-150 |
| | | | flammable | | negative | |
| HTC3D5 | WF3D5 | <200 | Non- | <3 | Ames- | 50-150 |
| | | | flammable | | negative | |
| HTC3D6 | WF3D6 | <200 | Non- | <3 | Ames- | 50-150 |
| | | | flammable | | negative | |
| HTC3D7 | WF3D7 | <200 | Non- | <3 | Ames- | 50-150 |
| | | | flammable | | negative | |
| HTC4-1 | WF4-1 | <200 | Non- | <3 | Ames- | 50-150 |
| | | | flammable | | negative | |
| HTC4-2 | WF4-2 | <200 | Non- | <3 | Ames- | 50-150 |
| | | | flammable | | negative | |
| HTC4-3 | WF4-3 | <200 | Non- | <3 | Ames- | 50-150 |
| | | | flammable | | negative | |
| HTC4-4 | WF4-4 | <200 | Non- | <3 | Ames- | 50-150 |
| | | | flammable | | negative | |
| HTC4-5 | WF4-5 | <200 | Non- | <3 | Ames- | 50-150 |
| | | | flammable | | negative | |
| HTC4-6 | WF4-6 | <200 | Non- | <3 | Ames- | 50-150 |
| | | | flammable | | negative | |
| HTC4-7 | WF4-7 | <200 | Non- | <3 | Ames- | 50-150 |
| | | | flammable | | negative | |
| HTC4A1 | WF4A1 | <200 | Non- | <3 | Ames- | 50-150 |
| | | | flammable | | negative | |
| HTC4A2 | WF4A2 | <200 | Non- | <3 | Ames- | 50-150 |
| | | | flammable | | negative | |
| HTC4A3 | WF4A3 | <200 | Non- | <3 | Ames- | 50-150 |
| | | | flammable | | negative | |
| HTC4A4 | WF4A4 | <200 | Non- | <3 | Ames- | 50-150 |
| | | | flammable | | negative | |
| HTC4A5 | WF4A5 | <200 | Non- | <3 | Ames- | 50-150 |
| | | | flammable | | negative | |
| HTC4A6 | WF4A6 | <200 | Non- | <3 | Ames- | 50-150 |
| | | | flammable | | negative | |
| HTC4A7 | WF4A7 | <200 | Non- | <3 | Ames- | 50-150 |
| | | | flammable | | negative | |
| HTC4B1 | WF4B1 | <200 | Non- | <3 | Ames- | 50-150 |
| | | | flammable | | negative | |
| HTC4B2 | WF4B2 | <200 | Non- | <3 | Ames- | 50-150 |
| | | | flammable | | negative | |
| HTC4B3 | WF4B3 | <200 | Non- | <3 | Ames- | 50-150 |
| | | | flammable | | negative | |
| HTC4B4 | WF4B4 | <200 | Non- | <3 | Ames- | 50-150 |
| | | | flammable | | negative | |
| HTC4B5 | WF4B5 | <200 | Non- | <3 | Ames- | 50-150 |
| | | | flammable | | negative | |
| HTC4B6 | WF4B6 | <200 | Non- | <3 | Ames- | 50-150 |
| | | | flammable | | negative | |
| HTC4B7 | WF4B7 | <200 | Non- | <3 | Ames- | 50-150 |
| | | | flammable | | negative | |
| HTC4C1 | WF4C1 | <200 | Non- | <3 | Ames- | 50-150 |
| | | | flammable | | negative | |
| HTC4C2 | WF4C2 | <200 | Non- | <3 | Ames- | 50-150 |
| | | | flammable | | negative | |
| HTC4C3 | WF4C3 | <200 | Non- | <3 | Ames- | 50-150 |
| | | | flammable | | negative | |
| HTC4C4 | WF4C4 | <200 | Non- | <3 | Ames- | 50-150 |
| | | | flammable | | negative | |
| HTC4C5 | WF4C5 | <200 | Non- | <3 | Ames- | 50-150 |
| | | | flammable | | negative | |
| HTC4C6 | WF4C6 | <200 | Non- | <3 | Ames- | 50-150 |
| | | | flammable | | negative | |
| HTC4C7 | WF4C7 | <200 | Non- | <3 | Ames- | 50-150 |
| | | | flammable | | negative | |
| HTC4D1 | WF4D1 | <200 | Non- | <3 | Ames- | 50-150 |
| | | | flammable | | negative | |
| HTC4D2 | WF4D2 | <200 | Non- | <3 | Ames- | 50-150 |
| | | | flammable | | negative | |
| HTC4D3 | WF4D3 | <200 | Non- | <3 | Ames- | 50-150 |
| | | | flammable | | negative | |
| HTC4D4 | WF4D4 | <200 | Non- | <3 | Ames- | 50-150 |
| | | | flammable | | negative | |
| HTC4D5 | WF4D5 | <200 | Non- | <3 | Ames- | 50-150 |
| | | | flammable | | negative | |
| HTC4D6 | HTC4D6 | <200 | Non- | <3 | Ames- | 50-150 |
| | | | flammable | | negative | |
| HTC4D7 | WFC4D7 | <200 | Non- | <3 | Ames- | 50-150 |
| | | | flammable | | negative | |
| HTC5-1 | WFC5-1 | <200 | Non- | <3 | Ames- | 50-150 |
| | | | flammable | | negative | |
| HTC5-2 | WFC5-2 | <200 | Non- | <3 | Ames- | 50-150 |
| | | | flammable | | negative | |
| HTC5-3 | WF5-3 | <200 | Non- | <3 | Ames- | 50-150 |
| | | | flammable | | negative | |
| HTC5-4 | WF5-4 | <200 | Non- | <3 | Ames- | 50-150 |
| | | | flammable | | negative | |
| HTC5-5 | WF5-5 | <200 | Non- | <3 | Ames- | 50-150 |
| | | | flammable | | negative | |
| HTC5-6 | WF5-6 | <200 | Non- | <3 | Ames- | 50-150 |
| | | | flammable | | negative | |
| HTC5-7 | WF5-7 | <200 | Non- | <3 | Ames- | 50-150 |
| | | | flammable | | negative | |
| HTC6-1 | WF6-1 | <200 | Non- | <3 | Ames- | 50-150 |
| | | | flammable | | negative | |
| HTC6-2 | WF6-2 | <200 | Non- | <3 | Ames- | 50-150 |
| | | | flammable | | negative | |
| HTC6-3 | WF6-3 | <200 | Non- | <3 | Ames- | 50-150 |
| | | | flammable | | negative | |
| HTC6-4 | WF6-4 | <200 | Non- | <3 | Ames- | 50-150 |
| | | | flammable | | negative | |
| HTC6-5 | WF6-5 | <200 | Non- | <3 | Ames- | 50-150 |
| | | | flammable | | negative | |
| HTC6-6 | WF6-6 | <200 | Non- | <3 | Ames- | 50-150 |
| | | | flammable | | negative | |
| HTC6-7 | WF6-7 | <200 | Non- | <3 | Ames- | 50-150 |
| | | | flammable | | negative | |

The present invention includes heat transfer compositions, including each of Compositions 1-6, and each of the heat transfer compositions in Table 3 above, namely HTC1-HTC6, wherein the co-heat transfer agent is selected from the group consisting of hexafluoroisopropylethylether, hexafluoroisopropylmethylthioether, HFE-7000, HFE-7200, HFE-7100, HFE-7300, HFE-7500, HFE-7600, trans-1,2-dichloroethylene, n-pentane, cyclopentane, ethanol, perfluoro(2-methyl-3-pentanone) (Novec 1230), cis-HFO-1336mzz, trans-HFO-1336mzz, HF-1234yf, HFO-1234ze(E), HFO-1233zd(E) and HFO-1233zd(Z).

In preferred embodiments, the compositions of the present invention, including each of Compositions 1-6, and each of the heat transfer compositions in Table 3 above, namely HTC1-HTC6, have a GWP of less than about 100.

In preferred embodiments, the compositions of the present invention, including each of Compositions 1-6, and each of the heat transfer compositions in Table 3 above, namely HTC1-HTC6, have a dielectric constant of less than 3.

In preferred embodiments, the compositions of the present invention, including each of Compositions 1-6, and each of the heat transfer compositions in Table 3 above, namely HTC1-HTC6, has a dielectric strength of at least about 30.

In preferred embodiments, the compositions of the present invention, including each of Compositions 1-6, and each of the heat transfer compositions in Table 3 above, namely HTC1-HTC6, has a dielectric strength of at least about 40.

In preferred embodiments, the compositions of the present invention, including each of Compositions 1-6 and each of the heat transfer compositions in Table 3 above, namely HTC1-HTC6, has a thermal conductivity of at least about 0.055 W/m-K.

In preferred embodiments, the compositions of the present invention, including each of Compositions 1-6 and each of the heat transfer compositions in Table 3 above, namely HTC1-HTC6, has a thermal conductivity of at least about 0.065 W/m-K.

Preferably, the heat transfer composition of the present invention, including each of Compositions 1-17 and 18A, and each of HTC1-HTC6 further comprises a lubricant. The lubricant lubricates the refrigeration compressor using the refrigerant. The lubricant may be present in the heat transfer composition in amounts of from about 5% to about 30% by weight of heat transfer composition. Lubricants such as Polyol Esters (POEs), Poly Alkylene Glycols (PAGs), PAG oils, polyvinyl ethers (PVEs), poly(alpha-olefin) (PAO), alkyl benzene and mineral oil and combinations thereof may be used in the heat transfer compositions of the present invention.

Preferred lubricants include POEs and PVEs, more preferably POEs, especially for use in connection with heat transfer methods comprising stationary air conditioning and refrigeration. Of course, different mixtures of different types of lubricants may be used. For example, the lubricant may be a PAG if the refrigerant is used in mobile air conditioning applications.

Commercially available POEs include neopentyl glycol dipelargonate which is available as Emery 2917 (registered trademark) and Hatcol 2370 (registered trademark) and pentaerythritol derivatives including those sold under the trade designations Emkarate RL32-3MAF and Emkarate RL68H by CPI Fluid Engineering. Emkarate RL32-3MAF and Emkarate RL68H are preferred neopently POE lubricants having the properties identified below:

***OMIT***

| Property | RL32-3MAF | RL68H |
|---|---|---|
| Viscosity | about 31 | about 67 |
| @ 40° C. (ASTM D467), cSt | | |
| Viscosity | about 5.6 | about 9.4 |
| @ 100° C. (ASTM D467), cSt | | |
| Pour Point | about −40 | about −40 |
| (ASTM D97), ° C. | | |

The lubricant of the present invention can include PVE lubricants generally. In preferred embodiments the PVE lubricant is as PVE according to Formula II below:

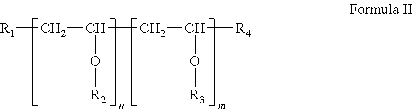

where R2 and R3 are each independently C1-C10 hydrocarbons, preferably C2-C8 hydrocarbons, and R1 and R4 are each independently alkyl, alkylene glycol, or polyoxyalkylene glycol units and n and m are selected preferably according to the needs of those skilled in the art to obtain a lubricant with the desired properties, and preferable n and m are selected to obtain a lubricant with a viscosity at 40° C. measured in accordance with ASTM D467 of from about 30 to about 70 cSt. Commercially available polyvinyl ethers include those lubricants sold under the trade designations FVC32D and FVC68D, from Idemitsu.

The heat transfer compositions therefore comprise in preferred embodiments any of the heat transfer compositions of the present invention, including each of HTC1-HTC6, and a lubricant selected from a POE, a PAG or a PVE.

The heat transfer composition of the present invention may consist essentially of or consist of a heat transfer fluid and lubricant as described above.

Commercially available mineral oils include Witco LP 250 (registered trademark) from Witco, Zerol 300 (registered trademark) from Shrieve Chemical, Sunisco 3GS from Witco, and Calumet R015 from Calumet. Commercially available alkyl benzene lubricants include Zerol 150 (registered trademark). Commercially available esters include neopentyl glycol dipelargonate, which is available as Emery 2917 (registered trademark) and Hatcol 2370 (registered trademark). Other useful esters include phosphate esters, dibasic acid esters, and fluoroesters.

The heat transfer composition may include a compatibilizer for the purpose of aiding compatibility and/or solubility of the lubricant. Suitable compatibilizers may include propane, butanes, pentanes, and/or hexanes. When present, the compatibilizer is preferably present in an amount of from about 0.5% to about 5% by weight of the heat transfer composition. Combinations of surfactants and solubilizing agents may also be added to the present compositions to aid oil solubility, as disclosed by U.S. Pat. No. 6,516,837, the disclosure of which is incorporated by reference.

### Thermal Management Fluids

One important category of heat transfer fluid according to the present invention is thermal management fluid. Accordingly, the present invention provides various methods, processes and uses of the compounds of the present invention, including each of Compounds 1-6 and the compositions of the present invention, including Compositions 1-6, and each of the heat transfer compositions in Table 3 above, namely HTC1-HTC6, as thermal management fluids (hereinafter sometimes referred to as TMFs) that are used help maintain an article or device (preferably an electronic device or battery) or fluid within a certain temperature range, particularly as that article, device or fluid is operating according to its intended purpose. For example, the TMFs compositions may be used to keep the temperature of a device below a defined upper and/or above a defined lower temperature.

The present invention includes each of the heat transfer compositions in Table 3 above, namely HTC1-HTC6, TMFs in accordance with the above Table 3 in which the co-TMF is selected from the group consisting of exafluoroisopropylethylether, hexafluoroisopropylmethylthioether, HFE-7000, HFE-7200, HFE-7100, HFE-7300, HFE-7500, HFE-7600, trans-1,2-dichloroethylene, n-pentane, cyclopentane, ethanol, perfluoro(2-methyl-3-pentanone) (Novec 1230), cis-HFO-1336mzz, trans-HFO-1336mzz, HF-1234yf, HFO-1234ze(E), HFO-1233zd(E) and HFO-1233zd(Z).

### Heat Transfer Uses, Methods, Systems and Devices

The present invention includes method for transferring heat as described herein, including methods as specifically described above and hereinafter.

The present invention also includes devices and systems for transferring heat as described herein, including devices and systems as specifically described above and hereinafter.

The heat transfer fluid, thermal management fluid, refrigerant, working fluid and heat transfer compositions, including each of Compositions 1-6, and each of the heat transfer compositions in Table 3 above, namely HTC1-HTC6, of the invention are provided for use for heating and/or cooling as described herein.

Thus, the present invention describes a method of heating or cooling a fluid or body using a heat transfer fluid, thermal management fluid, refrigerant, working fluid or heat transfer compositions of the invention, including each of Compositions 1-6, and each of the heat transfer compositions in Table 3 above, namely HTC1-HTC6.

Thermal Management Methods, Devices, Systems and Uses

In nearly every modern application of electronics, the dissipation of heat is an important consideration. For example, in portable and hand-held devices, the desire to miniaturize while adding functionality increases the thermal power density, which increases the challenge of cooling the electronics within them. As computational power increases within desktop computers, datacenters and telecommunications centers, so does the heat output. Power electronic devices such as the traction inverters in plug-in electric or hybrid vehicles, wind turbines, train engines, generators and various industrial processes make use of transistors that operate at ever higher currents and heat fluxes.

As discussed above, when a heat transfer fluid of the present invention, including each of Compositions 1-6, and each of the heat transfer compositions in Table 3 above, namely HTC1-HTC6, is used in a method or device or system of cooling and/or heating in an electronic device, it is sometimes referred to herein as a thermal management fluid. The thermal management fluid therefore corresponds to the heat transfer fluid as discussed in this application.

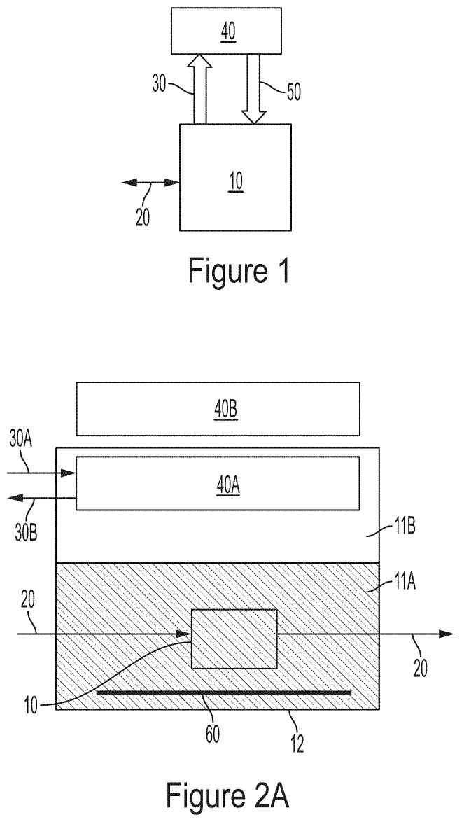

Preferred embodiments of the present thermal management methods, including Heat Transfer Methods 1 and 2, will now be described in connection with FIG. 1 in which an operating electronic device is shown schematically as 10 having a source of electrical energy and/or signals 20 flowing into and/or out of the device 10 and which generates heat as a result of its operation based on the electrical energy and/or signals 20. The thermal management fluid of the present invention is provided in thermal contact with the operating device 10 such that it removes heat, represented by the out flowing arrow 30. Heat is removed from the operating electronic device by sensible heat being added to the liquid thermal management fluid of the present invention (i.e., increasing the temperature of the liquid), or by causing a phase change in the thermal management liquid (i.e., vaporizing the liquid) or a combination of these. In preferred embodiments, the methods provide a supply of TMF of the present invention, including each of Compositions 1-6, and each of the heat transfer compositions in Table 3 above, namely HTC1-HTC6, to the device 10 such that the flow of heat from the device 10 through the present heat transfer fluid 30 maintains the operating electrical device at or within a preferred operating temperature range. In preferred embodiments, the preferred operating temperature range of the electrical device is from about 70° C. to about 150° C., and even more preferably from about 70° C. to about 120° C., and the flow of heat 30 from the device 10 through the present heat transfer fluid energy maintains the operating electrical device at or within such preferred temperature ranges. Preferably, the TMF 30 of the present invention, which has absorbed heat from the device, is in thermal contact with a heat sink, represented schematically as 40, at a temperature below the temperature of the heat transfer fluid 30 and thereby transfers the heat generated by the device 10 to the heat sink 40. In this way, the heat-depleted heat transfer fluid of the present invention 50 can be returned to the electronic device 10 to repeat the cycle of cooling.

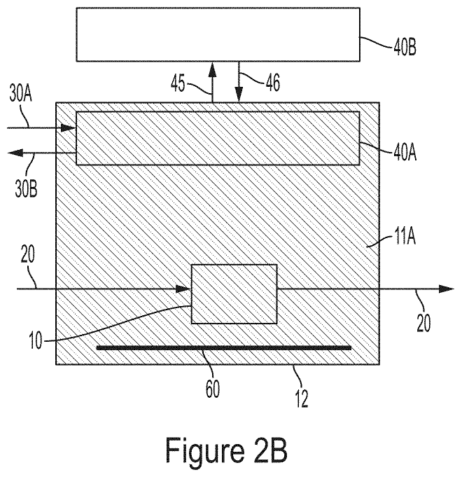

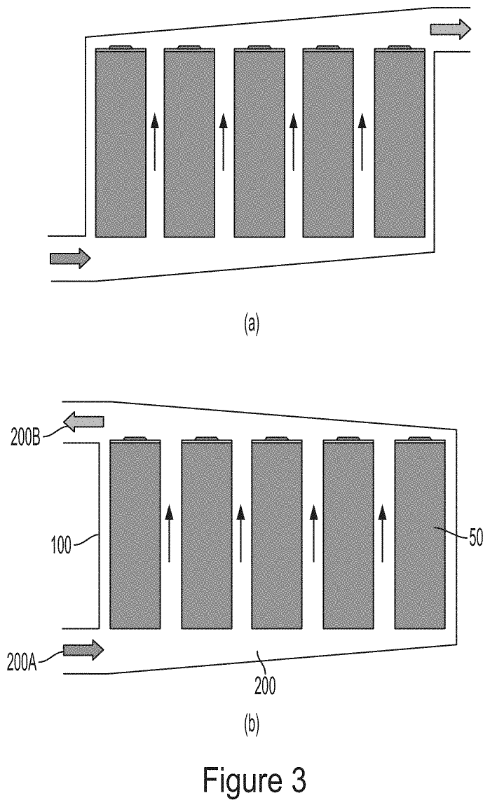

In a preferred embodiment of the present methods, the step of removing heat through a heat transfer composition of the present invention, including each of Compositions 1-6, and each of the heat transfer compositions in Table 3 above, namely HTC1-HTC6, comprises evaporating the heat transfer composition of the present invention using the heat generated by the operation of the electronic device, and the step of transferring that heat from the heat transfer composition to the heat sink comprises condensing the heat transfer fluid by rejecting heat to the heat sink. In such methods, the temperature of the heat transfer fluid of the present invention, including each of Compositions 1-6, and each of the heat transfer compositions in Table 3 above, namely HTC1-HTC6, during said evaporation step is preferably greater than 50° C., or preferably greater than about 55° C., or preferably in the range of from about 55° C. to about 85° C., or preferably from about 65° C. to about 75° C. Applicants have found that the present TMFs, including each of Compositions 1-6, and each of the heat transfer compositions in Table 3 above, namely HTC1-HTC6, provide excellent performance in such methods and at the same time allow the use of relatively low cost, lightweight and reliable equipment to provide the necessary cooling, as will be explained further in connection with particular embodiments as described in connection with FIG. 2A below.

In a further preferred embodiment of the present methods, the step of removing heat through the present heat transfer composition, including each of Compositions 1-6, and each of the heat transfer compositions in Table 3 above, namely HTC1-HTC6, comprises adding sensible heat to the liquid heat transfer composition of the present invention (e.g., raising the temperature of the liquid up to about 70° C. or less at about atmospheric pressure, i.e., wherein the fluid is not required to be in a high pressure container or vessel) using the heat generated by the operation of the electronic device, and the step of transferring that heat from the heat transfer composition to a heat sink and thereby reducing the liquid temperature by rejecting heat to the heat sink. The cooled liquid is then returned to thermal contact with the electrical device wherein the cycle starts over. In preferred embodiments, the temperature of the heat transfer liquid that is used to transfer heat to the heat sink is greater than about 40° C., or preferably greater than about 55° C., or preferably in the range of from about 45° C. to about 70° C., or preferably from about 45° C. to about 65° C., and preferably is at a pressure that is about atmospheric. Applicants have found that the present heat transfer liquids, including each of Compositions 1-6, and each of the heat transfer compositions in Table 3 above, namely HTC1-HTC6, provide excellent performance in such methods and at the same time allow the use for relatively low cost, lightweight and reliable equipment to provide the necessary cooling, as will be explained further in connection with particular embodiments as described in connection with FIG. 2B below.

It will be appreciated by those skilled in the art that the present invention comprises systems and methods which use both sensible heat transfer and phase change heat transfer as describe above.

A particular method according to the present invention will now be described in connection with FIGS. 2A and 2B in which an electronic device 10 is contained in an appropriate container 12, and preferably a sealed container, and is in direct contact with, and preferably fully immersed in liquid heat transfer composition of the present invention 11A (shown schematically by gray shading), including each of Compositions 1-6, and each of the heat transfer compositions in Table 3 above, namely HTC1-HTC6. For the purposes of convenience, such cooling methods, devices and system are sometimes referred to herein as “immersion cooling” methods, devices and systems.

In immersion cooling methods, devices and systems used to cool electrical devices or components, the operating electronic device 10 has a source of electrical energy and/or signals 20 flowing into and/or out of the container 12 and into and/or out of device 10, which generates heat as a result of its operation based on the electrical energy and/or signals 20. As those skilled in the art will appreciate, it is a significant challenge to discover a heat transfer fluid that can perform effectively in such applications since the fluid must not only provide all of the other properties mentioned above, but it must also be able to do so while in intimate contact with an operating electronic device, that is, one which involves the flow of electrical current/signals. It will be appreciated that many fluids that might be otherwise viable for use in such applications will not be useable because they will either short-out the device, degrade when exposed to the conditions created by the operation of the electronic device (i.e., degrade the cooling effect over time and/or the operating stability of the device), or have some other property detrimental to operation when in contact with an operating electronic device.

In contrast, the present methods produce excellent and unexpected results by providing the thermal management fluid of the present invention, including each of Compositions 1-6, and each of the heat transfer compositions in Table 3 above, namely HTC1-HTC6, in direct thermal and physical contact with the device 10 as it is operating. This heat of operation is safely and effectively transferred to the thermal management fluid 11A, including each of Compositions 1-6, and each of the heat transfer compositions in Table 3 above, namely HTC1-HTC6, by: (a) causing the liquid phase of the fluid to evaporate and form vapor 11B; or (b) raising the temperature of the liquid thermal management fluid 11A; or (c) a combination of (a) and (b).

When the thermal management fluid is a single-phase liquid, it will remain liquid when heated by the heat-generating component. Thus, the thermal management fluid can be brought into contact with the heat generating component, resulting in the removal of the heat from the heat generating component and the production of a thermal management fluid with a higher temperature. The thermal management fluid is then transported to a secondary cooling loop, such as a radiator or another refrigerated system. An example of such a system is illustrated in FIG. 2, where the thermal management fluid enters a battery pack enclosure containing a number of cells and exits the enclosure having taken up heat from the battery pack.

When the thermal management fluid of the present invention, including each of Compositions 1-6, and each of the heat transfer compositions in Table 3 above, namely HTC1-HTC6, is present in two phases, the heat-generating component is in thermal contact with the thermal management fluid and transfers heat to the thermal management fluid, resulting in the boiling of the thermal management fluid. The thermal management fluid is then condensed. An example of such a system is where the heat-generating component is immersed in the thermal management fluid and an external cooling circuit condenses the boiling fluid into a liquid state.

In the case of the phase change heat transfer systems of the present invention, reference is made herein to FIG. 2A. In such an operation, heat is carried away from the device 10 as the liquid evaporates and the vapor rises through the remaining thermal management liquid, including each of Compositions 1-6, and each of the heat transfer compositions in Table 3 above, namely HTC1-HTC6, in the container 12. The thermal management fluid vapor 11B then rejects the heat it has absorbed to a heat sink 40, which can be an enclosed heat sink 40A and/or an external heat sink 40B. An example of a heat sink that is internal to the container 12 are condenser coils 30A and 30B with circulating liquid, such as water, at a temperature below the condensing temperature of the thermal management fluid vapor. An example of a heat sink that is external to the container 12 would be passing relatively cool ambient air over the container 12 (which preferably in such case include cooling fins or the like), which will serve to condense the heat transfer vapor 11B on the interior surface of the container. As a result of this condensation, liquid thermal management fluid is returned to the pool of liquid fluid 11A in which the device 10 remains immersed in operation.

In the case of a sensible heat transfer systems of the present invention, reference is made herein to FIG. 2B. In such an operation heat is carried away from the device as the temperature of liquid 11A, including each of Compositions 1-6, and each of the heat transfer compositions in Table 3 above, namely HTC1-HTC6, increases upon accepting heat being generated by the device, which is immersed, and preferably substantially fully immersed in the thermal management fluid 11A of the present invention. The higher temperature thermal management fluid liquid 11A then rejects the heat it has absorbed to a heat sink 40, which can be an enclosed heat sink 40A and/or an external heat sink 40B. An example of a heat sink that is internal to the container 12 are cooling coils 30A and 30B with circulating liquid, such as water, at a temperature below the temperature of heated liquid. An example of a heat sink that is external to the container 12 would be removing heated liquid 11A from the container through a conduit 45 where it is thermally contacted with a cool fluid, such as might be provided by relatively cool ambient air, or cooled water or refrigerant, which will serve to lower the temperature of the liquid. Cooled liquid is then returned via conduit 46.

Optionally, but preferably in certain embodiments involving thermal management of the batteries used in electronic vehicles, the thermal management system includes a heating element which is able to heat the thermal management fluid, including each of Compositions 1-6, and each of the heat transfer compositions in Table 3 above, namely HTC1-HTC6, such as for example an electrical heating element 60 which is also immersed in the thermal management fluid. As those skilled in the art will appreciate, the batteries in electronic vehicles (which would correspond to the operating electronic device 10 in FIGS. 2A and 2B) can reach relatively low temperatures while parked outside in the winter months in many geographical locations, and frequently such low temperature conditions are not desirable for battery operation. Accordingly, the thermal management system of the present invention can include sensors and control modules (not shown) which turn on the heating element when the battery temperature is below a predetermined level. In such a case, the heater 60 would be activated, the thermal management liquid 11A would be heated, and would in turn transfer this heat to the electronic device 10 until the minimum temperature is reached. Thereafter during operation, the thermal management fluid of the present invention, including each of Compositions 1-6, and each of the heat transfer compositions in Table 3 above, namely HTC1-HTC6, would serve the cooling function as described above.

For the purposes of this invention, the thermal management fluid, including each of Compositions 1-6, and each of the heat transfer compositions in Table 3 above, namely HTC1-HTC6, can be in direct contact with the heat-generating component or in indirect contact with the heat-generating component.

When the thermal management fluid, including each of Compositions 1-6, and each of the heat transfer compositions in Table 3 above, namely HTC1-HTC6, is in indirect contact with the heat-generating component, the thermal management fluid can be used in a closed system in the electronic device, which may include at least two heat exchangers. When the thermal management fluid is used to cool the heat-generating component, heat can be transferred from the component to the thermal management fluid, usually through a heat exchanger in contact with at least a part of the component or the heat can be transferred to circulating air which can conduct the heat to a heat exchanger that is in thermal contact with the thermal management fluid.

In a particularly preferred feature of the present invention, the thermal management fluid, including each of Compositions 1-6, and each of the heat transfer compositions in Table 3 above, namely HTC1-HTC6, is in direct contact with the heat-generating component. In particular, the heat generating component is fully or partially immersed in the thermal management fluid. Preferably the heat generating component is fully immersed in the thermal management fluid, including each of Compositions 1-6, and each of the heat transfer compositions in Table 3 above, namely HTC1-HTC6. The thermal management fluid, as a warmed fluid or as a vapor, can then be circulated to a heat exchanger which takes the heat from the fluid or vapor and transfers it to the outside environment by way of a heat sink such as ambient air or water cooled by ambient air or otherwise. After this heat transfer, the cooled thermal management fluid (cooled or condensed) is recycled back into the system to cool the heat-generating component.

Electrical conductivity and/or dielectric strength of a thermal management fluid becomes important if the fluid comes in direct contact with the electronic components of the electronic device (such as in direct immersion cooling), or if the thermal management fluid leaks out of a cooling loop or is spilled during maintenance and comes in contact with the electrical circuits. Thus, the thermal management fluid of the present invention, including each of including each of Compositions 1-6, and each of the heat transfer compositions in Table 3 above, namely HTC1-HTC6, is preferably an electrically insulating thermal management fluid.

The thermal management fluid of the present invention, including each of including each of Compositions 1-6, and each of the heat transfer compositions in Table 3 above, namely HTC1-HTC6, may be recirculated passively or actively in the device, for example by using mechanical equipment such as a pump. In a preferred feature of the present invention, the thermal management fluid of the present invention, including each of including each of Compositions 1-6, and each of the heat transfer compositions in Table 3 above, namely HTC1-HTC6, is recirculated passively in the device.

Passive recirculating systems work by transferring heat from the heat-generating component to the thermal management fluid until it typically is vaporized, allowing the heated vapor to proceed to a heat exchange surface at which it transfers its heat to the heat exchanger surface and condenses back into a liquid. It will be appreciated that the heat exchange surface can be part of a separate heat exchange unit and/or can be integral with the container, as described above for example in connection with FIG. 2. The condensed liquid then returns, preferably fully passively by the force of gravity and/or a wicking structure, into the thermal management fluid in contact with the heat-generating component. Thus, in a preferred feature of the invention, the step of transferring heat from the heat-generating component to the thermal management fluid of the present invention, including each of including each of Compositions 1-6, and each of the heat transfer compositions in Table 3 above, namely HTC1-HTC6, causes the thermal management fluid to vaporize.

Examples of passive recirculating systems include a heat pipe or a thermosyphon. Such systems passively recirculate the thermal management fluid of the present invention, including each of including each of Compositions 1-6, and each of the heat transfer compositions in Table 3 above, namely HTC1-HTC6, using gravity. In such a system, the thermal management fluid is heated by the heat-generating component, resulting in a heated thermal management fluid which is less dense and more buoyant. This thermal management fluid travels to a storage container, such as a tank where it cools and condenses. The cooled thermal management fluid then flows back to the heat source.

The present invention includes use of the present compounds, including each of Compositions 1-6, and each of the heat transfer compositions in Table 3 above, namely HTC1-HTC6, to cool and optionally heat electronic devices that produce or include a component that is a heat-generating component. The heat-generating component can be any component that includes an electronic element that generates heat as part of its operation. For the purposes of this invention, the heat generating component includes but is not limited to: semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, circuit boards, multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

For the purpose of this invention, the electronic device includes but is not limited to: personal computers, microprocessors, servers, cell phones, tablets, digital home appliances (e.g., televisions, media players, games consoles etc.), personal digital assistants, datacenters, batteries both stationary and in vehicles, including Li-ion batteries and other batteries used in hybrid or electric vehicles, wind turbine, train engine, or generator. Preferably the electronic device is a hybrid or electric vehicle.

The present invention further relates to an electronic device comprising a thermal management fluid of the invention, including each of including each of Compositions 1-6, and each of the heat transfer compositions in Table 3 above, namely HTC1-HTC6. For the purposes of this invention, the thermal management fluid is provided for cooling and/or heating the electronic device.

The present invention further relates to an electronic device comprising a heat generating component and a thermal management fluid of the invention, including each of including each of Compositions 1-6, and each of the heat transfer compositions in Table 3 above, namely HTC1-HTC6, for cooling, and optionally heating, the electronic device.

The present invention further relates to an electronic device comprising a heat generating component, a heat exchanger, a pump and a thermal management fluid of the invention, including each of including each of Compositions 1-6, and each of the heat transfer compositions in Table 3 above, namely HTC1-HTC6. For the purpose of this invention, the electronic device can be any such device, including but not limited to personal computers, microprocessors, servers, cell phones, tablets, digital home appliances (e.g. televisions, media players, games consoles etc.), personal digital assistants, datacenters, hybrid or electric vehicles, batteries both stationary and in vehicles, electrical drive motors, fuel cells (e.g., hydrogen fuel cells) and electrical generators, preferably wherein the electronic device is in a hybrid vehicle, or electric vehicle, or wind turbine, or train.

For the purposes of this invention, the heat generating component can be any electrical component that generates heat during operation, but preferably electronic components that generate heat at high levels of heat flux. Examples of heat generating components that can be cooled according to the present invention include semiconductor integrated circuits (ICs), electrochemical cells, power transistors, resistors, and electroluminescent elements, such as microprocessors, wafers used to manufacture semiconductor devices, power control semiconductors, electrical distribution switch gear, power transformers, printed circuit boards (PCBs), multi-chip modules, packaged or unpackaged semiconductor devices, semiconductor integrated circuits, fuel cells, lasers (conventional or laser diodes), light emitting diodes (LEDs), and electrochemical cells, e.g. used for high power applications such as, for example, hybrid or electric vehicles.

Lithium-Ion Battery Cooling System

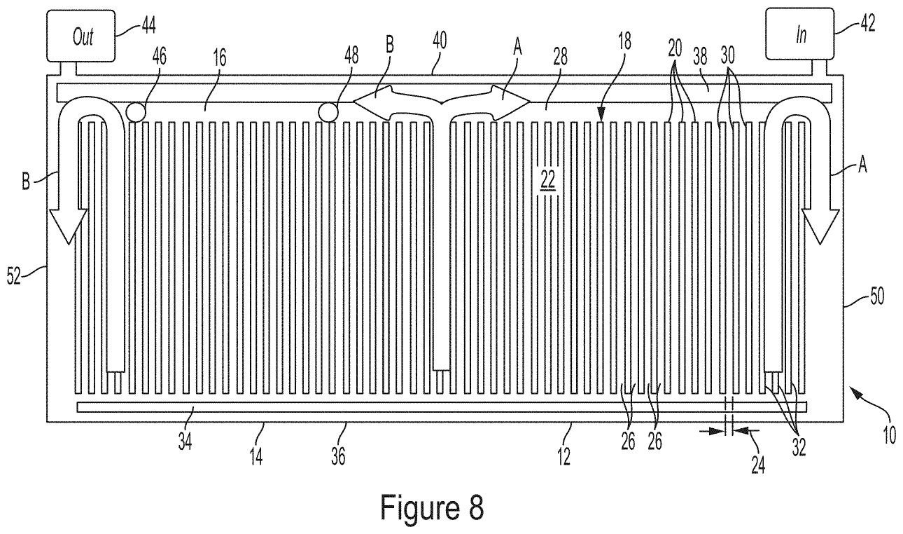

Examples of the present thermal management methods useful for lithium-ion battery cooling, including Heat Transfer Methods 1 and 2 and Thermal Management Methods 1-2, will now be described in connection with FIG. 8. A vehicle battery pack with a self-contained liquid cooling system 10 comprising a module 12 formed of a container 14 with an interior space 16 for supporting a battery assembly 18. The container 14 is a closed and sealed container 14 for forming a self-contained liquid cooling system 10. The battery assembly 18 includes a plurality of battery cells 20 such as a plurality of Lithium-ion (Li-ion) batteries for use in a hybrid vehicle. In another embodiment, the plurality of battery cells 20 is Li-ion batteries for use in a Battery Electric Vehicle (BEV). Additional batteries for use with other prime mover vehicles may be provided with the liquid cooling system 10 of the present invention, where each battery cell includes active material for generating power from an electrochemical reaction within the interior space 16 of the container 14. The battery cells 20 are preferably stacked to form a battery cell stack 22. In the embodiment shown, a gap 24 between each battery cell 20 is between 0.25-0.50 mm, forming a fluid channel 26 between each battery cell 20. In another embodiment, the gap 24 may be less than 0.25 mm. It is understood that other gap sizes can be used as desired.

A composition of the present invention, including each of Compositions 1-6, and each of the heat transfer compositions in Table 3 above, namely HTC1-HTC6, is disposed within the interior space 16 of the container 14 and the fluid level shown is such that the battery assembly 18 is completely immersed within the composition of the present invention. The composition of the present invention, including each of including each of Compositions 1-6, and each of the heat transfer compositions in Table 3 above, namely HTC1-HTC6, is in contact with the battery cells 20 through the fluid channels 26 formed by gaps 24.

A heating element 34 is located at a base area 36 of the container 14. The heating element 34 shown is an electronic heating element. It is understood that other heating element types may be used. The heating element 34 is shown as a single element; however, multiple heating elements 34 such as heating plates may be provided.

A cooling element 38 is located at an upper area 40 of the container 14. The cooling element 38 may be a chilled water condenser having an inlet 42 and an outlet 44 extending beyond the walls of the sealed container 14 for importing and exporting water for the cooling element 38. In another embodiment, the cooling element 38 may be a chilled water plate. In still another embodiment, the cooling element 38 may be a thin aluminum heat sink having external chilled water travelling through the cooling element 38. The cooling element 38 may be a graphite foil impregnated with an electrically nonconductive polymer. The cooling element may also be formed from copper.

In the embodiment shown, arrows “A” and “B” indicate a flow 28 of the composition of the present invention, including each of including each of Compositions 1-6, and each of the heat transfer compositions in Table 3 above, namely HTC1-HTC6. Upon heating of each battery cell 20 by the heating element 34, the coolant 28 of the present invention, including each of including each of Compositions 1-6, and each of the heat transfer compositions in Table 3 above, namely HTC1-HTC6, is exposed to a front surface area 30 and a rear surface area 32 of the battery cells 20, and will boil. The heated coolant 28 will rise and flow to the top of the battery cell stack 22 to be cooled by the cooling element 38. The cooled coolant 28 will return to the base area 36, generally following either coolant paths “A” or “B.” Where the general location of the coolant 28 at the moment of boiling is located within the fluid channels 26 of the battery cells 20 in the center area and toward a side 50 of the container 14, the coolant 28 will tend to follow flow path “A”. Similarly, if the general location of the dielectric coolant 28 at the moment of boiling is located within the fluid channels 26 of the battery cells 20 in the center area and toward an opposing side 52 of the container 14, the dielectric coolant 28 will tend to follow flow path “B”.

A coolant temperature sensor 46 is located on or near the cooling element 38. In the embodiment shown, the temperature sensor 46 is located within the area of the outlet 44 of the cooling element 38 and measures a temperature of the dielectric coolant 28 of the present invention at a point of exposure to the cooling element. The temperature sensor 46 may be located anywhere within the battery cell stack 22 as desired.

A coolant level sensor 48 is also provided and is located near the upper area 40 of the container 14 to measure the fluid level of the dielectric coolant 28 within the container 14, ensuring complete immersion of the battery assembly 18 within the dielectric coolant 28.

While the above description of cooling by immersion in a composition of the present invention, including each of Compositions 1-6, and each of the heat transfer compositions in Table 3 above, namely HTC1-HTC6, is made in connection with cooling of a batter, the same basic procedure, and all variations thereof within the skill of the art, can be used to cool any electronic component or device, as described herein, including each of those devices identified in Table 4 below.

The following Table 4 identifies preferred electronic devices and components that are cooled according to immersion cooling systems and methods or the present invention (with reference to NR indicating that there is no requirement associated with that feature and the reference to TMF being to the Thermal Management Fluids defined by number herein):

TABLE 3
PREFERRED IMMERSION COOLING
SYSTEMS AND METHODS

| | Device/ | | Operating | Evap. | Condensing |
|---|---|---|---|---|---|
| HTC | Electronic | Located | Temp, | Temp, | Temp, |
| No.* | Component | in | ° C. | ° C. | ° C. |
| HTC1 | Battery | NR | NR | NR | NR |
| HTC2 | Battery | NR | NR | NR | NR |
| HTC3 | Battery | NR | NR | NR | NR |
| HTC4 | Battery | NR | NR | NR | NR |
| HTC5 | Battery | NR | NR | NR | NR |
| HTC6 | Battery | NR | NR | NR | NR |
| HTC1 | Battery | NR | NR | >40 | NR |
| HTC2 | Battery | NR | NR | >40 | NR |
| HTC3 | Battery | NR | NR | >40 | NR |
| HTC4 | Battery | NR | NR | >40 | NR |
| HTC5 | Battery | NR | NR | >40 | NR |
| HTC6 | Battery | NR | NR | >40 | NR |
| HTC1 | Battery | NR | NR | 40-85 | NR |
| HTC2 | Battery | NR | NR | 40-85 | NR |
| HTC3 | Battery | NR | NR | 40-85 | NR |
| HTC4 | Battery | NR | NR | 40-85 | NR |
| HTC5 | Battery | NR | NR | 40-85 | NR |
| HTC6 | Battery | NR | NR | 40-85 | NR |
| HTC1 | Battery | NR | NR | 65-75 | NR |
| HTC2 | Battery | NR | NR | 65-75 | NR |
| HTC3 | Battery | NR | NR | 65-75 | NR |
| HTC4 | Battery | NR | NR | 65-75 | NR |
| HTC5 | Battery | NR | NR | 65-75 | NR |
| HTC6 | Battery | NR | NR | 65-75 | NR |
| HTC1 | Battery | NR | NR | >40 | 45-65 |
| HTC2 | Battery | NR | NR | >40 | 45-65 |
| HTC3 | Battery | NR | NR | >40 | 45-65 |
| HTC4 | Battery | NR | NR | >40 | 45-65 |
| HTC5 | Battery | NR | NR | >40 | 45-65 |
| HTC6 | Battery | NR | NR | >40 | 45-65 |
| HTC1 | Battery | NR | NR | 40-85 | 45-65 |
| HTC2 | Battery | NR | NR | 40-85 | 45-65 |
| HTC3 | Battery | NR | NR | 40-85 | 45-65 |
| HTC4 | Battery | NR | NR | 40-85 | 45-65 |
| HTC5 | Battery | NR | NR | 40-85 | 45-65 |
| HTC6 | Battery | NR | NR | 40-85 | 45-65 |
| HTC1 | Battery | NR | NR | 65-75 | 45-65 |
| HTC2 | Battery | NR | NR | 65-75 | 45-65 |
| HTC3 | Battery | NR | NR | 65-75 | 45-65 |
| HTC4 | Battery | NR | NR | 65-75 | 45-65 |
| HTC5 | Battery | NR | NR | 65-75 | 45-65 |
| HTC6 | Battery | NR | NR | 65-75 | 45-65 |
| HTC1 | Battery | NR | 50-150 | NR | NR |
| HTC2 | Battery | NR | 50-150 | NR | NR |
| HTC3 | Battery | NR | 50-150 | NR | NR |
| HTC4 | Battery | NR | 50-150 | NR | NR |
| HTC5 | Battery | NR | 50-150 | NR | NR |
| HTC6 | Battery | NR | 50-150 | NR | NR |
| HTC1 | Battery | NR | 50-150 | >40 | NR |
| HTC2 | Battery | NR | 50-150 | >40 | NR |
| HTC3 | Battery | NR | 50-150 | >40 | NR |
| HTC4 | Battery | NR | 50-150 | >40 | NR |
| HTC5 | Battery | NR | 50-150 | >40 | NR |
| HTC6 | Battery | NR | 50-150 | >40 | NR |
| HTC1 | Battery | NR | 50-150 | 40-85 | NR |
| HTC2 | Battery | NR | 50-150 | 40-85 | NR |
| HTC3 | Battery | NR | 50-150 | 40-85 | NR |
| HTC4 | Battery | NR | 50-150 | 40-85 | NR |
| HTC5 | Battery | NR | 50-150 | 40-85 | NR |
| HTC6 | Battery | NR | 50-150 | 40-85 | NR |
| HTC1 | Battery | NR | 50-150 | 65-75 | NR |
| HTC2 | Battery | NR | 50-150 | 65-75 | NR |
| HTC3 | Battery | NR | 50-150 | 65-75 | NR |
| HTC4 | Battery | NR | 50-150 | 65-75 | NR |
| HTC5 | Battery | NR | 50-150 | 65-75 | NR |
| HTC6 | Battery | NR | 50-150 | 65-75 | NR |
| HTC1 | Battery | NR | 50-150 | >40 | 45-65 |
| HTC2 | Battery | NR | 50-150 | >40 | 45-65 |
| HTC3 | Battery | NR | 50-150 | >40 | 45-65 |
| HTC4 | Battery | NR | 50-150 | >40 | 45-65 |
| HTC5 | Battery | NR | 50-150 | >40 | 45-65 |
| HTC6 | Battery | NR | 50-150 | >40 | 45-65 |
| HTC1 | Battery | NR | 50-150 | 40-85 | 45-65 |
| HTC2 | Battery | NR | 50-150 | 40-85 | 45-65 |
| HTC3 | Battery | NR | 50-150 | 40-85 | 45-65 |
| HTC4 | Battery | NR | 50-150 | 40-85 | 45-65 |
| HTC5 | Battery | NR | 50-150 | 40-85 | 45-65 |
| HTC6 | Battery | NR | 50-150 | 40-85 | 45-65 |
| HTC1 | Battery | NR | 50-150 | 65-75 | 45-65 |
| HTC2 | Battery | NR | 50-150 | 65-75 | 45-65 |
| HTC3 | Battery | NR | 50-150 | 65-75 | 45-65 |
| HTC4 | Battery | NR | 50-150 | 65-75 | 45-65 |
| HTC5 | Battery | NR | 50-150 | 65-75 | 45-65 |
| HTC6 | Battery | NR | 50-150 | 65-75 | 45-65 |
| HTC1 | Battery | NR | 70-120 | NR | NR |
| HTC2 | Battery | NR | 70-120 | NR | NR |
| HTC3 | Battery | NR | 70-120 | NR | NR |
| HTC4 | Battery | NR | 70-120 | NR | NR |
| HTC5 | Battery | NR | 70-120 | NR | NR |
| HTC6 | Battery | NR | 70-120 | NR | NR |
| HTC1 | Battery | NR | 70-120 | >40 | NR |
| HTC2 | Battery | NR | 70-120 | >40 | NR |
| HTC3 | Battery | NR | 70-120 | >40 | NR |
| HTC4 | Battery | NR | 70-120 | >40 | NR |
| HTC5 | Battery | NR | 70-120 | >40 | NR |
| HTC6 | Battery | NR | 70-120 | >40 | NR |
| HTC1 | Battery | NR | 70-120 | 40-85 | NR |
| HTC2 | Battery | NR | 70-120 | 40-85 | NR |
| HTC3 | Battery | NR | 70-120 | 40-85 | NR |
| HTC4 | Battery | NR | 70-120 | 40-85 | NR |
| HTC5 | Battery | NR | 70-120 | 40-85 | NR |
| HTC6 | Battery | NR | 70-120 | 40-85 | NR |
| HTC1 | Battery | NR | 70-120 | 65-75 | NR |
| HTC2 | Battery | NR | 70-120 | 65-75 | NR |
| HTC3 | Battery | NR | 70-120 | 65-75 | NR |
| HTC4 | Battery | NR | 70-120 | 65-75 | NR |
| HTC5 | Battery | NR | 70-120 | 65-75 | NR |
| HTC6 | Battery | NR | 70-120 | 65-75 | NR |
| HTC1 | Battery | NR | 70-120 | >40 | 45-65 |
| HTC2 | Battery | NR | 70-120 | >40 | 45-65 |
| HTC3 | Battery | NR | 70-120 | >40 | 45-65 |
| HTC4 | Battery | NR | 70-120 | >40 | 45-65 |
| HTC5 | Battery | NR | 70-120 | >40 | 45-65 |
| HTC6 | Battery | NR | 70-120 | >40 | 45-65 |
| HTC1 | Battery | NR | 70-120 | 40-85 | 45-65 |
| HTC2 | Battery | NR | 70-120 | 40-85 | 45-65 |
| HTC3 | Battery | NR | 70-120 | 40-85 | 45-65 |
| HTC4 | Battery | NR | 70-120 | 40-85 | 45-65 |
| HTC5 | Battery | NR | 70-120 | 40-85 | 45-65 |
| HTC6 | Battery | NR | 70-120 | 40-85 | 45-65 |
| HTC1 | Battery | NR | 70-120 | 65-75 | 45-65 |
| HTC2 | Battery | NR | 70-120 | 65-75 | 45-65 |
| HTC3 | Battery | NR | 70-120 | 65-75 | 45-65 |
| HTC4 | Battery | NR | 70-120 | 65-75 | 45-65 |
| HTC5 | Battery | NR | 70-120 | 65-75 | 45-65 |
| HTC6 | Battery | NR | 70-120 | 65-75 | 45-65 |
| HTC1 | Battery | Electric | NR | NR | NR |
| | | Vehicle | | | |
| HTC2 | Battery | Electric | NR | NR | NR |
| | | Vehicle | | | |
| HTC3 | Battery | Electric | NR | NR | NR |
| | | Vehicle | | | |
| HTC4 | Battery | Electric | NR | NR | NR |
| | | Vehicle | | | |
| HTC5 | Battery | Electric | NR | NR | NR |
| | | Vehicle | | | |
| HTC6 | Battery | Electric | NR | NR | NR |
| | | Vehicle | | | |
| HTC1 | Battery | Electric | NR | NR | NR |
| | | Vehicle | | | |
| HTC2 | Battery | Electric | NR | NR | NR |
| | | Vehicle | | | |
| HTC3 | Battery | Electric | NR | NR | NR |
| | | Vehicle | | | |
| HTC4 | Battery | Electric | NR | NR | NR |
| | | Vehicle | | | |
| HTC5 | Battery | Electric | NR | NR | NR |
| | | Vehicle | | | |
| HTC6 | Battery | Electric | NR | NR | NR |
| | | Vehicle | | | |
| HTC1 | Battery | Electric | NR | 40-85 | NR |
| | | Vehicle | | | |
| HTC2 | Battery | Electric | NR | 40-85 | NR |
| | | Vehicle | | | |
| HTC3 | Battery | Electric | NR | 40-85 | NR |
| | | Vehicle | | | |
| HTC4 | Battery | Electric | NR | 40-85 | NR |
| | | Vehicle | | | |
| HTC5 | Battery | Electric | NR | 40-85 | NR |
| | | Vehicle | | | |
| HTC6 | Battery | Electric | NR | 40-85 | NR |
| | | Vehicle | | | |
| HTC1 | Battery | Electric | NR | 65-75 | NR |
| | | Vehicle | | | |
| HTC2 | Battery | Electric | NR | 65-75 | NR |
| | | Vehicle | | | |
| HTC3 | Battery | Electric | NR | 65-75 | NR |
| | | Vehicle | | | |
| HTC4 | Battery | Electric | NR | 65-75 | NR |
| | | Vehicle | | | |
| HTC5 | Battery | Electric | NR | 65-75 | NR |
| | | Vehicle | | | |
| HTC6 | Battery | Electric | NR | 65-75 | NR |
| | | Vehicle | | | |
| HTC1 | Battery | Electric | NR | >40 | 45-65 |
| | | Vehicle | | | |
| HTC2 | Battery | Electric | NR | >40 | 45-65 |
| | | Vehicle | | | |
| HTC3 | Battery | Electric | NR | >40 | 45-65 |
| | | Vehicle | | | |
| HTC4 | Battery | Electric | NR | >40 | 45-65 |
| | | Vehicle | | | |
| HTC5 | Battery | Electric | NR | >40 | 45-65 |
| | | Vehicle | | | |
| HTC1 | Battery | Electric | NR | 40-85 | 45-65 |
| | | Vehicle | | | |
| HTC2 | Battery | Electric | NR | 40-85 | 45-65 |
| | | Vehicle | | | |
| HTC3 | Battery | Electric | NR | 40-85 | 45-65 |
| | | Vehicle | | | |
| HTC4 | Battery | Electric | NR | 40-85 | 45-65 |
| | | Vehicle | | | |
| HTC5 | Battery | Electric | NR | 40-85 | 45-65 |
| | | Vehicle | | | |
| HTC6 | Battery | Electric | NR | 40-85 | 45-65 |
| | | Vehicle | | | |
| HTC1 | Battery | Electric | NR | 65-75 | 45-65 |
| | | Vehicle | | | |
| HTC2 | Battery | Electric | NR | 65-75 | 45-65 |
| | | Vehicle | | | |
| HTC3 | Battery | Electric | NR | 65-75 | 45-65 |
| | | Vehicle | | | |
| HTC4 | Battery | Electric | NR | 65-75 | 45-65 |
| | | Vehicle | | | |
| HTC5 | Battery | Electric | NR | 65-75 | 45-65 |
| | | Vehicle | | | |
| HTC6 | Battery | Electric | NR | 65-75 | 45-65 |
| | | Vehicle | | | |
| HTC1 | Battery | Electric | 50-150 | NR | NR |
| | | Vehicle | | | |
| HTC2 | Battery | Electric | 50-150 | NR | NR |
| | | Vehicle | | | |
| HTC3 | Battery | Electric | 50-150 | NR | NR |
| | | Vehicle | | | |
| HTC4 | Battery | Electric | 50-150 | NR | NR |
| | | Vehicle | | | |
| HTC5 | Battery | Electric | 50-150 | NR | NR |
| | | Vehicle | | | |
| HTC6 | Battery | Electric | 50-150 | NR | NR |
| | | Vehicle | | | |
| HTC1 | Battery | Electric | 50-150 | >40 | NR |
| | | Vehicle | | | |
| HTC2 | Battery | Electric | 50-150 | >40 | NR |
| | | Vehicle | | | |
| HTC3 | Battery | Electric | 50-150 | >40 | NR |
| | | Vehicle | | | |
| HTC4 | Battery | Electric | 50-150 | >40 | NR |
| | | Vehicle | | | |
| HTC5 | Battery | Electric | 50-150 | >40 | NR |
| | | Vehicle | | | |
| HTC6 | Battery | Electric | 50-150 | >40 | NR |
| | | Vehicle | | | |
| HTC1 | Battery | Electric | 50-150 | 40-85 | NR |
| | | Vehicle | | | |
| HTC2 | Battery | Electric | 50-150 | 40-85 | NR |
| | | Vehicle | | | |
| HTC3 | Battery | Electric | 50-150 | 40-85 | NR |
| | | Vehicle | | | |
| HTC4 | Battery | Electric | 50-150 | 40-85 | NR |
| | | Vehicle | | | |
| HTC5 | Battery | Electric | 50-150 | 40-85 | NR |
| | | Vehicle | | | |
| HTC6 | Battery | Electric | 50-150 | 40-85 | NR |
| | | Vehicle | | | |
| HTC1 | Battery | Electric | 50-150 | 65-75 | NR |
| | | Vehicle | | | |
| HTC2 | Battery | Electric | 50-150 | 65-75 | NR |
| | | Vehicle | | | |
| HTC3 | Battery | Electric | 50-150 | 65-75 | NR |
| | | Vehicle | | | |
| HTC4 | Battery | Electric | 50-150 | 65-75 | NR |
| | | Vehicle | | | |
| HTC5 | Battery | Electric | 50-150 | 65-75 | NR |
| | | Vehicle | | | |
| HTC6 | Battery | Electric | 50-150 | 65-75 | NR |
| | | Vehicle | | | |
| HTC1 | Battery | Electric | 50-150 | >40 | 45-65 |
| | | Vehicle | | | |
| HTC2 | Battery | Electric | 50-150 | >40 | 45-65 |
| | | Vehicle | | | |
| HTC3 | Battery | Electric | 50-150 | >40 | 45-65 |
| | | Vehicle | | | |
| HTC4 | Battery | Electric | 50-150 | >40 | 45-65 |
| | | Vehicle | | | |
| HTC5 | Battery | Electric | 50-150 | >40 | 45-65 |
| | | Vehicle | | | |
| HTC6 | Battery | Electric | 50-150 | >40 | 45-65 |
| | | Vehicle | | | |
| HTC1 | Battery | Electric | 50-150 | 40-85 | 45-65 |
| | | Vehicle | | | |
| HTC2 | Battery | Electric | 50-150 | 40-85 | 45-65 |
| | | Vehicle | | | |
| HTC3 | Battery | Electric | 50-150 | 40-85 | 45-65 |
| | | Vehicle | | | |
| HTC4 | Battery | Electric | 50-150 | 40-85 | 45-65 |
| | | Vehicle | | | |
| HTC5 | Battery | Electric | 50-150 | 40-85 | 45-65 |
| | | Vehicle | | | |
| HTC6 | Battery | Electric | 50-150 | 40-85 | 45-65 |
| | | Vehicle | | | |
| HTC1 | Battery | Electric | 50-150 | 65-75 | 45-65 |
| | | Vehicle | | | |
| HTC2 | Battery | Electric | 50-150 | 65-75 | 45-65 |
| | | Vehicle | | | |
| HTC3 | Battery | Electric | 50-150 | 65-75 | 45-65 |
| | | Vehicle | | | |
| HTC4 | Battery | Electric | 50-150 | 65-75 | 45-65 |
| | | Vehicle | | | |
| HTC5 | Battery | Electric | 50-150 | 65-75 | 45-65 |
| | | Vehicle | | | |
| HTC6 | Battery | Electric | 50-150 | 65-75 | 45-65 |
| | | Vehicle | | | |
| HTC1 | Battery | Electric | 70-120 | NR | NR |
| | | Vehicle | | | |
| HTC2 | Battery | Electric | 70-120 | NR | NR |
| | | Vehicle | | | |
| HTC3 | Battery | Electric | 70-120 | NR | NR |
| | | Vehicle | | | |
| HTC4 | Battery | Electric | 70-120 | NR | NR |
| | | Vehicle | | | |
| HTC5 | Battery | Electric | 70-120 | NR | NR |
| | | Vehicle | | | |
| HTC6 | Battery | Electric | 70-120 | NR | NR |
| | | Vehicle | | | |
| HTC1 | Battery | Electric | 70-120 | >40 | NR |
| | | Vehicle | | | |
| HTC2 | Battery | Electric | 70-120 | >40 | NR |
| | | Vehicle | | | |
| HTC3 | Battery | Electric | 70-120 | >40 | NR |
| | | Vehicle | | | |
| HTC4 | Battery | Electric | 70-120 | >40 | NR |
| | | Vehicle | | | |
| HTC5 | Battery | Electric | 70-120 | >40 | NR |
| | | Vehicle | | | |
| HTC6 | Battery | Electric | 70-120 | >40 | NR |
| | | Vehicle | | | |
| HTC1 | Battery | Electric | 70-120 | >40 | NR |
| | | Vehicle | | | |
| HTC2 | Battery | Electric | 70-120 | >40 | NR |
| | | Vehicle | | | |
| HTC3 | Battery | Electric | 70-120 | >40 | NR |
| | | Vehicle | | | |
| HTC4 | Battery | Electric | 70-120 | >40 | NR |
| | | Vehicle | | | |
| HTC5 | Battery | Electric | 70-120 | >40 | NR |
| | | Vehicle | | | |
| HTC6 | Battery | Electric | 70-120 | >40 | NR |
| | | Vehicle | | | |
| HTC1 | Battery | Electric | 70-120 | 65-75 | NR |
| | | Vehicle | | | |
| HTC2 | Battery | Electric | 70-120 | 65-75 | NR |
| | | Vehicle | | | |
| HTC3 | Battery | Electric | 70-120 | 65-75 | NR |
| | | Vehicle | | | |
| HTC4 | Battery | Electric | 70-120 | 65-75 | NR |
| | | Vehicle | | | |
| HTC5 | Battery | Electric | 70-120 | 65-75 | NR |
| | | Vehicle | | | |
| HTC6 | Battery | Electric | 70-120 | 65-75 | NR |
| | | Vehicle | | | |
| HTC1 | Battery | Electric | 70-120 | >40 | 45-65 |
| | | Vehicle | | | |
| HTC2 | Battery | Electric | 70-120 | >40 | 45-65 |
| | | Vehicle | | | |
| HTC3 | Battery | Electric | 70-120 | >40 | 45-65 |
| | | Vehicle | | | |
| HTC4 | Battery | Electric | 70-120 | >40 | 45-65 |
| | | Vehicle | | | |
| HTC5 | Battery | Electric | 70-120 | >40 | 45-65 |
| | | Vehicle | | | |
| HTC6 | Battery | Electric | 70-120 | >40 | 45-65 |
| | | Vehicle | | | |
| HTC1 | Battery | Electric | 70-120 | 40-85 | 45-65 |
| | | Vehicle | | | |
| HTC2 | Battery | Electric | 70-120 | 40-85 | 45-65 |
| | | Vehicle | | | |
| HTC3 | Battery | Electric | 70-120 | 40-85 | 45-65 |
| | | Vehicle | | | |
| HTC4 | Battery | Electric | 70-120 | 40-85 | 45-65 |
| | | Vehicle | | | |
| HTC5 | Battery | Electric | 70-120 | 40-85 | 45-65 |
| | | Vehicle | | | |
| HTC6 | Battery | Electric | 70-120 | 40-85 | 45-65 |
| | | Vehicle | | | |
| HTC1 | Battery | Electric | 70-120 | 65-75 | 45-65 |
| | | Vehicle | | | |
| HTC2 | Battery | Electric | 70-120 | 65-75 | 45-65 |
| | | Vehicle | | | |
| HTC3 | Battery | Electric | 70-120 | 65-75 | 45-65 |
| | | Vehicle | | | |
| HTC4 | Battery | Electric | 70-120 | 65-75 | 45-65 |
| | | Vehicle | | | |
| HTC5 | Battery | Electric | 70-120 | 65-75 | 45-65 |
| | | Vehicle | | | |
| HTC6 | Battery | Electric | 70-120 | 65-75 | 45-65 |
| | | Vehicle | | | |
| HTC1 | Battery | Hybrid | NR | NR | NR |
| | | Vehicle | | | |
| HTC2 | Battery | Hybrid | NR | NR | NR |
| | | Vehicle | | | |
| HTC3 | Battery | Hybrid | NR | NR | NR |
| | | Vehicle | | | |
| HTC4 | Battery | Hybrid | NR | NR | NR |
| | | Vehicle | | | |
| HTC5 | Battery | Hybrid | NR | NR | NR |
| | | Vehicle | | | |
| HTC6 | Battery | Hybrid | NR | NR | NR |
| | | Vehicle | | | |
| HTC1 | Battery | Hybrid | NR | >40 | NR |
| | | Vehicle | | | |
| HTC2 | Battery | Hybrid | NR | >40 | NR |
| | | Vehicle | | | |
| HTC3 | Battery | Hybrid | NR | >40 | NR |
| | | Vehicle | | | |
| HTC4 | Battery | Hybrid | NR | >40 | NR |
| | | Vehicle | | | |
| HTC5 | Battery | Hybrid | NR | >40 | NR |
| | | Vehicle | | | |
| HTC6 | Battery | Hybrid | NR | >40 | NR |
| | | Vehicle | | | |
| HTC1 | Battery | Hybrid | NR | 40-85 | NR |
| | | Vehicle | | | |
| HTC2 | Battery | Hybrid | NR | 40-85 | NR |
| | | Vehicle | | | |
| HTC3 | Battery | Hybrid | NR | 40-85 | NR |
| | | Vehicle | | | |
| HTC4 | Battery | Hybrid | NR | 40-85 | NR |
| | | Vehicle | | | |
| HTC5 | Battery | Hybrid | NR | 40-85 | NR |
| | | Vehicle | | | |
| HTC6 | Battery | Hybrid | NR | 40-85 | NR |
| | | Vehicle | | | |
| HTC1 | Battery | Hybrid | NR | 65-75 | NR |
| | | Vehicle | | | |
| HTC2 | Battery | Hybrid | NR | 65-75 | NR |
| | | Vehicle | | | |
| HTC3 | Battery | Hybrid | NR | 65-75 | NR |
| | | Vehicle | | | |
| HTC4 | Battery | Hybrid | NR | 65-75 | NR |
| | | Vehicle | | | |
| HTC5 | Battery | Hybrid | NR | 65-75 | NR |
| | | Vehicle | | | |
| HTC6 | Battery | Hybrid | NR | 65-75 | NR |
| | | Vehicle | | | |
| HTC1 | Battery | Hybrid | NR | >40 | 45-65 |
| | | Vehicle | | | |
| HTC2 | Battery | Hybrid | NR | >40 | 45-65 |
| | | Vehicle | | | |
| HTC3 | Battery | Hybrid | NR | >40 | 45-65 |
| | | Vehicle | | | |
| HTC4 | Battery | Hybrid | NR | >40 | 45-65 |
| | | Vehicle | | | |
| HTC5 | Battery | Hybrid | NR | >40 | 45-65 |
| | | Vehicle | | | |
| HTC6 | Battery | Hybrid | NR | >40 | 45-65 |
| | | Vehicle | | | |
| HTC1 | Battery | Hybrid | NR | 40-85 | 45-65 |
| | | Vehicle | | | |
| HTC2 | Battery | Hybrid | NR | 40-85 | 45-65 |
| | | Vehicle | | | |
| HTC3 | Battery | Hybrid | NR | 40-85 | 45-65 |
| | | Vehicle | | | |
| HTC4 | Battery | Hybrid | NR | 40-85 | 45-65 |
| | | Vehicle | | | |
| HTC5 | Battery | Hybrid | NR | 40-85 | 45-65 |
| | | Vehicle | | | |
| HTC6 | Battery | Hybrid | NR | 40-85 | 45-65 |
| | | Vehicle | | | |
| HTC1 | Battery | Hybrid | NR | 65-75 | 45-65 |
| | | Vehicle | | | |
| HTC2 | Battery | Hybrid | NR | 65-75 | 45-65 |
| | | Vehicle | | | |
| HTC3 | Battery | Hybrid | NR | 65-75 | 45-65 |
| | | Vehicle | | | |
| HTC4 | Battery | Hybrid | NR | 65-75 | 45-65 |
| | | Vehicle | | | |
| HTC5 | Battery | Hybrid | NR | 65-75 | 45-65 |
| | | Vehicle | | | |
| HTC6 | Battery | Hybrid | NR | 65-75 | 45-65 |
| | | Vehicle | | | |
| HTC1 | Battery | Hybrid | 50-150 | NR | NR |
| | | Vehicle | | | |
| HTC2 | Battery | Hybrid | 50-150 | NR | NR |
| | | Vehicle | | | |
| HTC3 | Battery | Hybrid | 50-150 | NR | NR |
| | | Vehicle | | | |
| HTC4 | Battery | Hybrid | 50-150 | NR | NR |
| | | Vehicle | | | |
| HTC5 | Battery | Hybrid | 50-150 | NR | NR |
| | | Vehicle | | | |
| HTC6 | Battery | Hybrid | 50-150 | NR | NR |
| | | Vehicle | | | |
| HTC1 | Battery | Hybrid | 50-150 | >40 | NR |
| | | Vehicle | | | |
| HTC2 | Battery | Hybrid | 50-150 | >40 | NR |
| | | Vehicle | | | |
| HTC3 | Battery | Hybrid | 50-150 | >40 | NR |
| | | Vehicle | | | |
| HTC4 | Battery | Hybrid | 50-150 | >40 | NR |
| | | Vehicle | | | |
| HTC5 | Battery | Hybrid | 50-150 | >40 | NR |
| | | Vehicle | | | |
| HTC6 | Battery | Hybrid | 50-150 | >40 | NR |
| | | Vehicle | | | |
| HTC1 | Battery | Hybrid | 50-150 | 40-85 | NR |
| | | Vehicle | | | |
| HTC2 | Battery | Hybrid | 50-150 | 40-85 | NR |
| | | Vehicle | | | |
| HTC3 | Battery | Hybrid | 50-150 | 40-85 | NR |
| | | Vehicle | | | |
| HTC4 | Battery | Hybrid | 50-150 | 40-85 | NR |
| | | Vehicle | | | |
| HTC5 | Battery | Hybrid | 50-150 | 40-85 | NR |
| | | Vehicle | | | |
| HTC6 | Battery | Hybrid | 50-150 | 40-85 | NR |
| | | Vehicle | | | |
| HTC1 | Battery | Hybrid | 50-150 | 65-75 | NR |
| | | Vehicle | | | |
| HTC2 | Battery | Hybrid | 50-150 | 65-75 | NR |
| | | Vehicle | | | |
| HTC3 | Battery | Hybrid | 50-150 | 65-75 | NR |
| | | Vehicle | | | |
| HTC4 | Battery | Hybrid | 50-150 | 65-75 | NR |
| | | Vehicle | | | |
| HTC5 | Battery | Hybrid | 50-150 | 65-75 | NR |
| | | Vehicle | | | |
| HTC6 | Battery | Hybrid | 50-150 | 65-75 | NR |
| | | Vehicle | | | |
| HTC1 | Battery | Hybrid | 50-150 | >40 | 45-65 |
| | | Vehicle | | | |
| HTC2 | Battery | Hybrid | 50-150 | >40 | 45-65 |
| | | Vehicle | | | |
| HTC3 | Battery | Hybrid | 50-150 | >40 | 45-65 |
| | | Vehicle | | | |
| HTC4 | Battery | Hybrid | 50-150 | >40 | 45-65 |
| | | Vehicle | | | |
| HTC5 | Battery | Hybrid | 50-150 | >40 | 45-65 |
| | | Vehicle | | | |
| HTC6 | Battery | Hybrid | 50-150 | >40 | 45-65 |
| | | Vehicle | | | |
| HTC1 | Battery | Hybrid | 50-150 | 40-85 | 45-65 |
| | | Vehicle | | | |
| HTC2 | Battery | Hybrid | 50-150 | 40-85 | 45-65 |
| | | Vehicle | | | |
| HTC3 | Battery | Hybrid | 50-150 | 40-85 | 45-65 |
| | | Vehicle | | | |
| HTC4 | Battery | Hybrid | 50-150 | 40-85 | 45-65 |
| | | Vehicle | | | |
| HTC5 | Battery | Hybrid | 50-150 | 40-85 | 45-65 |
| | | Vehicle | | | |
| HTC6 | Battery | Hybrid | 50-150 | 40-85 | 45-65 |
| | | Vehicle | | | |
| HTC1 | Battery | Hybrid | 50-150 | 65-75 | 45-65 |
| | | Vehicle | | | |
| HTC2 | Battery | Hybrid | 50-150 | 65-75 | 45-65 |
| | | Vehicle | | | |
| HTC3 | Battery | Hybrid | 50-150 | 65-75 | 45-65 |
| | | Vehicle | | | |
| HTC4 | Battery | Hybrid | 50-150 | 65-75 | 45-65 |
| | | Vehicle | | | |
| HTC5 | Battery | Hybrid | 50-150 | 65-75 | 45-65 |
| | | Vehicle | | | |
| HTC6 | Battery | Hybrid | 50-150 | 65-75 | 45-65 |
| | | Vehicle | | | |
| HTC1 | Battery | Hybrid | 70-120 | NR | NR |
| | | Vehicle | | | |
| HTC2 | Battery | Hybrid | 70-120 | NR | NR |
| | | Vehicle | | | |
| HTC3 | Battery | Hybrid | 70-120 | NR | NR |
| | | Vehicle | | | |
| HTC4 | Battery | Hybrid | 70-120 | NR | NR |
| | | Vehicle | | | |
| HTC5 | Battery | Hybrid | 70-120 | NR | NR |
| | | Vehicle | | | |
| HTC6 | Battery | Hybrid | 70-120 | NR | NR |
| | | Vehicle | | | |
| HTC1 | | | | | |
| HTC1 | Battery | Hybrid | 70-120 | >40 | NR |
| | | Vehicle | | | |
| HTC2 | Battery | Hybrid | 70-120 | >40 | NR |
| | | Vehicle | | | |
| HTC3 | Battery | Hybrid | 70-120 | >40 | NR |
| | | Vehicle | | | |
| HTC4 | Battery | Hybrid | 70-120 | >40 | NR |
| | | Vehicle | | | |
| HTC5 | Battery | Hybrid | 70-120 | >40 | NR |
| | | Vehicle | | | |
| HTC6 | Battery | Hybrid | 70-120 | >40 | NR |
| | | Vehicle | | | |
| HTC1 | Battery | Hybrid | 70-120 | 40-85 | NR |
| | | Vehicle | | | |
| HTC2 | Battery | Hybrid | 70-120 | 40-85 | NR |
| | | Vehicle | | | |
| HTC3 | Battery | Hybrid | 70-120 | 40-85 | NR |
| | | Vehicle | | | |
| HTC4 | Battery | Hybrid | 70-120 | 40-85 | NR |
| | | Vehicle | | | |
| HTC5 | Battery | Hybrid | 70-120 | 40-85 | NR |
| | | Vehicle | | | |
| HTC6 | Battery | Hybrid | 70-120 | 40-85 | NR |
| | | Vehicle | | | |
| HTC1 | Battery | Hybrid | 70-120 | 65-75 | NR |
| | | Vehicle | | | |
| HTC1 | Battery | Hybrid | 70-120 | 65-75 | NR |
| | | Vehicle | | | |
| HTC2 | Battery | Hybrid | 70-120 | 65-75 | NR |
| | | Vehicle | | | |
| HTC3 | Battery | Hybrid | 70-120 | 65-75 | NR |
| | | Vehicle | | | |
| HTC4 | Battery | Hybrid | 70-120 | 65-75 | NR |
| | | Vehicle | | | |
| HTC5 | Battery | Hybrid | 70-120 | 65-75 | NR |
| | | Vehicle | | | |
| HTC6 | Battery | Hybrid | 70-120 | 65-75 | NR |
| | | Vehicle | | | |
| HTC1 | Battery | Hybrid | 70-120 | >40 | 45-65 |
| | | Vehicle | | | |
| HTC2 | Battery | Hybrid | 70-120 | >40 | 45-65 |
| | | Vehicle | | | |
| HTC3 | Battery | Hybrid | 70-120 | >40 | 45-65 |
| | | Vehicle | | | |
| HTC4 | Battery | Hybrid | 70-120 | >40 | 45-65 |
| | | Vehicle | | | |
| HTC5 | Battery | Hybrid | 70-120 | >40 | 45-65 |
| | | Vehicle | | | |
| HTC6 | Battery | Hybrid | 70-120 | >40 | 45-65 |
| | | Vehicle | | | |
| HTC1 | Battery | Hybrid | 70-120 | 40-85 | 45-65 |
| | | Vehicle | | | |
| HTC2 | Battery | Hybrid | 70-120 | 40-85 | 45-65 |
| | | Vehicle | | | |
| HTC3 | Battery | Hybrid | 70-120 | 40-85 | 45-65 |
| | | Vehicle | | | |
| HTC4 | Battery | Hybrid | 70-120 | 40-85 | 45-65 |
| | | Vehicle | | | |
| HTC5 | Battery | Hybrid | 70-120 | 40-85 | 45-65 |
| | | Vehicle | | | |
| HTC6 | Battery | Hybrid | 70-120 | 40-85 | 45-65 |
| | | Vehicle | | | |
| HTC1 | Battery | Hybrid | 70-120 | 65-75 | 45-65 |
| | | Vehicle | | | |
| HTC2 | Battery | Hybrid | 70-120 | 65-75 | 45-65 |
| | | Vehicle | | | |
| HTC3 | Battery | Hybrid | 70-120 | 65-75 | 45-65 |
| | | Vehicle | | | |
| HTC4 | Battery | Hybrid | 70-120 | 65-75 | 45-65 |
| | | Vehicle | | | |
| HTC5 | Battery | Hybrid | 70-120 | 65-75 | 45-65 |
| | | Vehicle | | | |
| HTC6 | Battery | Hybrid | 70-120 | 65-75 | 45-65 |
| | | Vehicle | | | |
| HTC1 | Electric | Electric | NR | NR | NR |
| | Drive Motor | Vehicle | | | |
| HTC2 | Electric | Electric | NR | NR | NR |
| | Drive Motor | Vehicle | | | |
| HTC3 | Electric | Electric | NR | NR | NR |
| | Drive Motor | Vehicle | | | |
| HTC4 | Electric | Electric | NR | NR | NR |
| | Drive Motor | Vehicle | | | |
| HTC5 | Electric | Electric | NR | NR | NR |
| | Drive Motor | Vehicle | | | |
| HTC6 | Electric | Electric | NR | NR | NR |
| | Drive Motor | Vehicle | | | |
| HTC1 | Electric | Electric | NR | >40 | NR |
| | Drive Motor | Vehicle | | | |
| HTC2 | Electric | Electric | NR | >40 | NR |
| | Drive Motor | Vehicle | | | |
| HTC3 | Electric | Electric | NR | >40 | NR |
| | Drive Motor | Vehicle | | | |
| HTC4 | Electric | Electric | NR | >40 | NR |
| | Drive Motor | Vehicle | | | |
| HTC5 | Electric | Electric | NR | >40 | NR |
| | Drive Motor | Vehicle | | | |
| HTC6 | Electric | Electric | NR | >40 | NR |
| | Drive Motor | Vehicle | | | |
| HTC1 | Electric | Electric | NR | 40-85 | NR |
| | Drive Motor | Vehicle | | | |
| HTC2 | Electric | Electric | NR | 40-85 | NR |
| | Drive Motor | Vehicle | | | |
| HTC3 | Electric | Electric | NR | 40-85 | NR |
| | Drive Motor | Vehicle | | | |
| HTC4 | Electric | Electric | NR | 40-85 | NR |
| | Drive Motor | Vehicle | | | |
| HTC5 | Electric | Electric | NR | 40-85 | NR |
| | Drive Motor | Vehicle | | | |
| HTC6 | Electric | Electric | NR | 40-85 | NR |
| | Drive Motor | Vehicle | | | |
| HTC1 | Electric | Electric | NR | 65-75 | NR |
| | Drive Motor | Vehicle | | | |
| HTC2 | Electric | Electric | NR | 65-75 | NR |
| | Drive Motor | Vehicle | | | |
| HTC3 | Electric | Electric | NR | 65-75 | NR |
| | Drive Motor | Vehicle | | | |
| HTC4 | Electric | Electric | NR | 65-75 | NR |
| | Drive Motor | Vehicle | | | |
| HTC5 | Electric | Electric | NR | 65-75 | NR |
| | Drive Motor | Vehicle | | | |
| HTC6 | Electric | Electric | NR | 65-75 | NR |
| | Drive Motor | Vehicle | | | |
| HTC1 | Electric | Electric | NR | >40 | 45-65 |
| | Drive Motor | Vehicle | | | |
| HTC2 | Electric | Electric | NR | >40 | 45-65 |
| | Drive Motor | Vehicle | | | |
| HTC3 | Electric | Electric | NR | >40 | 45-65 |
| | Drive Motor | Vehicle | | | |
| HTC4 | Electric | Electric | NR | >40 | 45-65 |
| | Drive Motor | Vehicle | | | |
| HTC5 | Electric | Electric | NR | >40 | 45-65 |
| | Drive Motor | Vehicle | | | |
| HTC6 | Electric | Electric | NR | >40 | 45-65 |
| | Drive Motor | Vehicle | | | |
| HTC1 | Electric | Electric | NR | 40-85 | 45-65 |
| | Drive Motor | Vehicle | | | |
| HTC2 | Electric | Electric | NR | 40-85 | 45-65 |
| | Drive Motor | Vehicle | | | |
| HTC3 | Electric | Electric | NR | 40-85 | 45-65 |
| | Drive Motor | Vehicle | | | |
| HTC4 | Electric | Electric | NR | 40-85 | 45-65 |
| | Drive Motor | Vehicle | | | |
| HTC5 | Electric | Electric | NR | 40-85 | 45-65 |
| | Drive Motor | Vehicle | | | |
| HTC6 | Electric | Electric | NR | 40-85 | 45-65 |
| | Drive Motor | Vehicle | | | |
| HTC1 | Electric | Electric | NR | 65-75 | 45-65 |
| | Drive Motor | Vehicle | | | |
| HTC2 | Electric | Electric | NR | 65-75 | 45-65 |
| | Drive Motor | Vehicle | | | |
| HTC3 | Electric | Electric | NR | 65-75 | 45-65 |
| | Drive Motor | Vehicle | | | |
| HTC4 | Electric | Electric | NR | 65-75 | 45-65 |
| | Drive Motor | Vehicle | | | |
| HTC5 | Electric | Electric | NR | 65-75 | 45-65 |
| | Drive Motor | Vehicle | | | |
| HTC6 | Electric | Electric | NR | 65-75 | 45-65 |
| | Drive Motor | Vehicle | | | |
| HTC1 | Electric | Hybrid | NR | NR | NR |
| | Drive Motor | Vehicle | | | |
| HTC2 | Electric | Hybrid | NR | NR | NR |
| | Drive Motor | Vehicle | | | |
| HTC3 | Electric | Hybrid | NR | NR | NR |
| | Drive Motor | Vehicle | | | |
| HTC4 | Electric | Hybrid | NR | NR | NR |
| | Drive Motor | Vehicle | | | |
| HTC5 | Electric | Hybrid | NR | NR | NR |
| | Drive Motor | Vehicle | | | |
| HTC6 | Electric | Hybrid | NR | NR | NR |
| | Drive Motor | Vehicle | | | |
| | Electric | Hybrid | NR | NR | NR |
| | Drive Motor | Vehicle | | | |
| HTC1 | Electric | Hybrid | NR | >40 | NR |
| | Drive Motor | Vehicle | | | |
| HTC2 | Electric | Hybrid | NR | >40 | NR |
| | Drive Motor | Vehicle | | | |
| HTC3 | Electric | Hybrid | NR | >40 | NR |
| | Drive Motor | Vehicle | | | |
| HTC4 | Electric | Hybrid | NR | >40 | NR |
| | Drive Motor | Vehicle | | | |
| HTC5 | Electric | Hybrid | NR | >40 | NR |
| | Drive Motor | Vehicle | | | |
| HTC6 | Electric | Hybrid | NR | >40 | NR |
| | Drive Motor | Vehicle | | | |
| HTC1 | Electric | Hybrid | NR | 40-85 | NR |
| | Drive Motor | Vehicle | | | |
| HTC2 | Electric | Hybrid | NR | 40-85 | NR |
| | Drive Motor | Vehicle | | | |
| HTC3 | Electric | Hybrid | NR | 40-85 | NR |
| | Drive Motor | Vehicle | | | |
| HTC4 | Electric | Hybrid | NR | 40-85 | NR |
| | Drive Motor | Vehicle | | | |
| HTC5 | Electric | Hybrid | NR | 40-85 | NR |
| | Drive Motor | Vehicle | | | |
| HTC6 | Electric | Hybrid | NR | 40-85 | NR |
| | Drive Motor | Vehicle | | | |
| HTC1 | Electric | Hybrid | NR | 65-75 | NR |
| | Drive Motor | Vehicle | | | |
| HTC2 | Electric | Hybrid | NR | 65-75 | NR |
| | Drive Motor | Vehicle | | | |
| HTC3 | Electric | Hybrid | NR | 65-75 | NR |
| | Drive Motor | Vehicle | | | |
| HTC4 | Electric | Hybrid | NR | 65-75 | NR |
| | Drive Motor | Vehicle | | | |
| HTC5 | Electric | Hybrid | NR | 65-75 | NR |
| | Drive Motor | Vehicle | | | |
| HTC6 | Electric | Hybrid | NR | 65-75 | NR |
| | Drive Motor | Vehicle | | | |
| HTC1 | Electric | Hybrid | NR | >40 | 45-65 |
| | Drive Motor | Vehicle | | | |
| HTC2 | Electric | Hybrid | NR | >40 | 45-65 |
| | Drive Motor | Vehicle | | | |
| HTC3 | Electric | Hybrid | NR | >40 | 45-65 |
| | Drive Motor | Vehicle | | | |
| HTC4 | Electric | Hybrid | NR | >40 | 45-65 |
| | Drive Motor | Vehicle | | | |
| HTC5 | Electric | Hybrid | NR | >40 | 45-65 |
| | Drive Motor | Vehicle | | | |
| HTC6 | Electric | Hybrid | NR | >40 | 45-65 |
| | Drive Motor | Vehicle | | | |
| HTC1 | Electric | Hybrid | NR | 40-85 | 45-65 |
| | Drive Motor | Vehicle | | | |
| HTC2 | Electric | Hybrid | NR | 40-85 | 45-65 |
| | Drive Motor | Vehicle | | | |
| HTC3 | Electric | Hybrid | NR | 40-85 | 45-65 |
| | Drive Motor | Vehicle | | | |
| HTC4 | Electric | Hybrid | NR | 40-85 | 45-65 |
| | Drive Motor | Vehicle | | | |
| HTC5 | Electric | Hybrid | NR | 40-85 | 45-65 |
| | Drive Motor | Vehicle | | | |
| HTC6 | Electric | Hybrid | NR | 40-85 | 45-65 |
| | Drive Motor | Vehicle | | | |
| HTC1 | Electric | Hybrid | NR | 65-75 | 45-65 |
| | Drive Motor | Vehicle | | | |
| HTC2 | Electric | Hybrid | NR | 65-75 | 45-65 |
| | Drive Motor | Vehicle | | | |
| HTC3 | Electric | Hybrid | NR | 65-75 | 45-65 |
| | Drive Motor | Vehicle | | | |
| HTC4 | Electric | Hybrid | NR | 65-75 | 45-65 |
| | Drive Motor | Vehicle | | | |
| HTC5 | Electric | Hybrid | NR | 65-75 | 45-65 |
| | Drive Motor | Vehicle | | | |
| HTC6 | Electric | Hybrid | NR | 65-75 | 45-65 |
| | Drive Motor | Vehicle | | | |
| HTC1 | PCB | NR | NR | NR | NR |
| HTC2 | PCB | NR | NR | NR | NR |
| HTC3 | PCB | NR | NR | NR | NR |
| HTC4 | PCB | NR | NR | NR | NR |
| HTC5 | PCB | NR | NR | NR | NR |
| HTC6 | PCB | NR | NR | NR | NR |
| HTC1 | PCB | NR | NR | >40 | NR |
| HTC2 | PCB | NR | NR | >40 | NR |
| HTC3 | PCB | NR | NR | >40 | NR |
| HTC4 | PCB | NR | NR | >40 | NR |
| HTC5 | PCB | NR | NR | >40 | NR |
| HTC6 | PCB | NR | NR | >40 | NR |
| HTC1 | PCB | NR | NR | 40-85 | NR |
| HTC2 | PCB | NR | NR | 40-85 | NR |
| HTC3 | PCB | NR | NR | 40-85 | NR |
| HTC4 | PCB | NR | NR | 40-85 | NR |
| HTC5 | PCB | NR | NR | 40-85 | NR |
| HTC6 | PCB | NR | NR | 40-85 | NR |
| HTC1 | PCB | NR | NR | 65-75 | NR |
| HTC2 | PCB | NR | NR | 65-75 | NR |
| HTC3 | PCB | NR | NR | 65-75 | NR |
| HTC4 | PCB | NR | NR | 65-75 | NR |
| HTC5 | PCB | NR | NR | 65-75 | NR |
| HTC6 | PCB | NR | NR | 65-75 | NR |
| HTC1 | PCB | NR | NR | >40 | 45-65 |
| HTC2 | PCB | NR | NR | >40 | 45-65 |
| HTC3 | PCB | NR | NR | >40 | 45-65 |
| HTC4 | PCB | NR | NR | >40 | 45-65 |
| HTC5 | PCB | NR | NR | >40 | 45-65 |
| HTC6 | PCB | NR | NR | >40 | 45-65 |
| HTC1 | PCB | NR | NR | 40-85 | 45-65 |
| HTC2 | PCB | NR | NR | 40-85 | 45-65 |
| HTC3 | PCB | NR | NR | 40-85 | 45-65 |
| HTC4 | PCB | NR | NR | 40-85 | 45-65 |
| HTC5 | PCB | NR | NR | 40-85 | 45-65 |
| HTC6 | PCB | NR | NR | 40-85 | 45-65 |
| | PCB | NR | NR | 40-85 | 45-65 |
| HTC1 | PCB | NR | NR | 65-75 | 45-65 |
| HTC2 | PCB | NR | NR | 65-75 | 45-65 |
| HTC3 | PCB | NR | NR | 65-75 | 45-65 |
| HTC4 | PCB | NR | NR | 65-75 | 45-65 |
| HTC5 | PCB | NR | NR | 65-75 | 45-65 |
| HTC6 | PCB | NR | NR | 65-75 | 45-65 |
| HTC1 | PCB | NR | 70-150 | NR | NR |
| HTC2 | PCB | NR | 70-150 | NR | NR |
| HTC3 | PCB | NR | 70-150 | NR | NR |
| HTC4 | PCB | NR | 70-150 | NR | NR |
| HTC5 | PCB | NR | 70-150 | NR | NR |
| HTC6 | PCB | NR | 70-150 | NR | NR |
| HTC1 | PCB | NR | 70-150 | >40 | NR |
| HTC2 | PCB | NR | 70-150 | >40 | NR |
| HTC3 | PCB | NR | 70-150 | >40 | NR |
| HTC4 | PCB | NR | 70-150 | >40 | NR |
| HTC5 | PCB | NR | 70-150 | >40 | NR |
| HTC6 | PCB | NR | 70-150 | >40 | NR |
| HTC1 | PCB | NR | 70-150 | 40-85 | NR |
| HTC2 | PCB | NR | 70-150 | 40-85 | NR |
| HTC3 | PCB | NR | 70-150 | 40-85 | NR |
| HTC4 | PCB | NR | 70-150 | 40-85 | NR |
| HTC5 | PCB | NR | 70-150 | 40-85 | NR |
| HTC6 | PCB | NR | 70-150 | 40-85 | NR |
| HTC1 | PCB | NR | 70-150 | 65-75 | NR |
| HTC2 | PCB | NR | 70-150 | 65-75 | NR |
| HTC3 | PCB | NR | 70-150 | 65-75 | NR |
| HTC4 | PCB | NR | 70-150 | 65-75 | NR |
| HTC5 | PCB | NR | 70-150 | 65-75 | NR |
| HTC6 | PCB | NR | 70-150 | 65-75 | NR |
| HTC1 | PCB | NR | 70-150 | >40 | 45-65 |
| HTC2 | PCB | NR | 70-150 | >40 | 45-65 |
| HTC3 | PCB | NR | 70-150 | >40 | 45-65 |
| HTC4 | PCB | NR | 70-150 | >40 | 45-65 |
| HTC5 | PCB | NR | 70-150 | >40 | 45-65 |
| HTC6 | PCB | NR | 70-150 | >40 | 45-65 |
| HTC1 | PCB | NR | 70-150 | 40-85 | 45-65 |
| HTC2 | PCB | NR | 70-150 | 40-85 | 45-65 |
| HTC3 | PCB | NR | 70-150 | 40-85 | 45-65 |
| HTC4 | PCB | NR | 70-150 | 40-85 | 45-65 |
| HTC5 | PCB | NR | 70-150 | 40-85 | 45-65 |
| HTC6 | PCB | NR | 70-150 | 40-85 | 45-65 |
| HTC1 | PCB | NR | 70-150 | 65-75 | 45-65 |
| HTC2 | PCB | NR | 70-150 | 65-75 | 45-65 |
| HTC3 | PCB | NR | 70-150 | 65-75 | 45-65 |
| HTC4 | PCB | NR | 70-150 | 65-75 | 45-65 |
| HTC5 | PCB | NR | 70-150 | 65-75 | 45-65 |
| HTC6 | PCB | NR | 70-150 | 65-75 | 45-65 |
| HTC1 | Super | NR | NR | NR | NR |
| | Computer | | | | |
| HTC2 | Super | NR | NR | NR | NR |
| | Computer | | | | |
| HTC3 | Super | NR | NR | NR | NR |
| | Computer | | | | |
| HTC4 | Super | NR | NR | NR | NR |
| | Computer | | | | |
| HTC5 | Super | NR | NR | NR | NR |
| | Computer | | | | |
| HTC6 | Super | NR | NR | NR | NR |
| | Computer | | | | |
| HTC1 | Super | NR | NR | >40 | NR |
| | Computer | | | | |
| HTC2 | Super | NR | NR | >40 | NR |
| | Computer | | | | |
| HTC3 | Super | NR | NR | >40 | NR |
| | Computer | | | | |
| HTC4 | Super | NR | NR | >40 | NR |
| | Computer | | | | |
| HTC5 | Super | NR | NR | >40 | NR |
| | Computer | | | | |
| HTC6 | Super | NR | NR | >40 | NR |
| | Computer | | | | |
| HTC1 | Super | NR | NR | 40-85 | NR |
| | Computer | | | | |
| HTC2 | Super | NR | NR | 40-85 | NR |
| | Computer | | | | |
| HTC3 | Super | NR | NR | 40-85 | NR |
| | Computer | | | | |
| HTC4 | Super | NR | NR | 40-85 | NR |
| | Computer | | | | |
| HTC5 | Super | NR | NR | 40-85 | NR |
| | Computer | | | | |
| HTC6 | Super | NR | NR | 40-85 | NR |
| | Computer | | | | |
| HTC1 | Super | NR | NR | 65-75 | NR |
| | Computer | | | | |
| HTC2 | Super | NR | NR | 65-75 | NR |
| | Computer | | | | |
| HTC3 | Super | NR | NR | 65-75 | NR |
| | Computer | | | | |
| HTC4 | Super | NR | NR | 65-75 | NR |
| | Computer | | | | |
| HTC5 | Super | NR | NR | 65-75 | NR |
| | Computer | | | | |
| HTC6 | Super | NR | NR | 65-75 | NR |
| | Computer | | | | |
| HTC1 | Super | NR | NR | >40 | 45-65 |
| | Computer | | | | |
| HTC2 | Super | NR | NR | >40 | 45-65 |
| | Computer | | | | |
| HTC3 | Super | NR | NR | >40 | 45-65 |
| | Computer | | | | |
| HTC4 | Super | NR | NR | >40 | 45-65 |
| | Computer | | | | |
| HTC5 | Super | NR | NR | >40 | 45-65 |
| | Computer | | | | |
| HTC6 | Super | NR | NR | >40 | 45-65 |
| | Computer | | | | |
| HTC1 | Super | NR | NR | 40-85 | 45-65 |
| | Computer | | | | |
| HTC2 | Super | NR | NR | 40-85 | 45-65 |
| | Computer | | | | |
| HTC3 | Super | NR | NR | 40-85 | 45-65 |
| | Computer | | | | |
| HTC4 | Super | NR | NR | 40-85 | 45-65 |
| | Computer | | | | |
| HTC5 | Super | NR | NR | 40-85 | 45-65 |
| | Computer | | | | |
| HTC6 | Super | NR | NR | 40-85 | 45-65 |
| | Computer | | | | |
| HTC1 | Super | NR | NR | 65-75 | 45-65 |
| | Computer | | | | |
| HTC2 | Super | NR | NR | 65-75 | 45-65 |
| | Computer | | | | |
| HTC3 | Super | NR | NR | 65-75 | 45-65 |
| | Computer | | | | |
| HTC4 | Super | NR | NR | 65-75 | 45-65 |
| | Computer | | | | |
| HTC5 | Super | NR | NR | 65-75 | 45-65 |
| | Computer | | | | |
| HTC6 | Super | NR | NR | 65-75 | 45-65 |
| | Computer | | | | |
| HTC1 | Super | NR | 70-150 | NR | NR |
| | Computer | | | | |
| HTC2 | Super | NR | 70-150 | NR | NR |
| | Computer | | | | |
| HTC3 | Super | NR | 70-150 | NR | NR |
| | Computer | | | | |
| HTC4 | Super | NR | 70-150 | NR | NR |
| | Computer | | | | |
| HTC5 | Super | NR | 70-150 | NR | NR |
| | Computer | | | | |
| HTC6 | Super | NR | 70-150 | NR | NR |
| | Computer | | | | |
| HTC1 | Super | NR | 70-150 | >40 | NR |
| | Computer | | | | |
| HTC2 | Super | NR | 70-150 | >40 | NR |
| | Computer | | | | |
| HTC3 | Super | NR | 70-150 | >40 | NR |
| | Computer | | | | |
| HTC4 | Super | NR | 70-150 | >40 | NR |
| | Computer | | | | |
| HTC5 | Super | NR | 70-150 | >40 | NR |
| | Computer | | | | |
| HTC6 | Super | NR | 70-150 | >40 | NR |
| | Computer | | | | |
| HTC1 | Super | NR | 70-150 | 40-85 | NR |
| | Computer | | | | |
| HTC2 | Super | NR | 70-150 | 40-85 | NR |
| | Computer | | | | |
| HTC3 | Super | NR | 70-150 | 40-85 | NR |
| | Computer | | | | |
| HTC4 | Super | NR | 70-150 | 40-85 | NR |
| | Computer | | | | |
| HTC5 | Super | NR | 70-150 | 40-85 | NR |
| | Computer | | | | |
| HTC6 | Super | NR | 70-150 | 40-85 | NR |
| | Computer | | | | |
| HTC1 | Super | NR | 70-150 | 65-75 | NR |
| | Computer | | | | |
| HTC2 | Super | NR | 70-150 | 65-75 | NR |
| | Computer | | | | |
| HTC3 | Super | NR | 70-150 | 65-75 | NR |
| | Computer | | | | |
| HTC4 | Super | NR | 70-150 | 65-75 | NR |
| | Computer | | | | |
| HTC5 | Super | NR | 70-150 | 65-75 | NR |
| | Computer | | | | |
| HTC6 | Super | NR | 70-150 | 65-75 | NR |
| | Computer | | | | |
| HTC1 | Super | NR | 70-150 | >40 | 45-65 |
| | Computer | | | | |
| HTC2 | Super | NR | 70-150 | >40 | 45-65 |
| | Computer | | | | |
| HTC3 | Super | NR | 70-150 | >40 | 45-65 |
| | Computer | | | | |
| HTC4 | Super | NR | 70-150 | >40 | 45-65 |
| | Computer | | | | |
| HTC5 | Super | NR | 70-150 | >40 | 45-65 |
| | Computer | | | | |
| HTC6 | Super | NR | 70-150 | >40 | 45-65 |
| | Computer | | | | |
| HTC1 | Super | NR | 70-120 | 40-85 | 45-65 |
| | Computer | | | | |
| HTC2 | Super | NR | 70-120 | 40-85 | 45-65 |
| | Computer | | | | |
| HTC3 | Super | NR | 70-120 | 40-85 | 45-65 |
| | Computer | | | | |
| HTC4 | Super | NR | 70-120 | 40-85 | 45-65 |
| | Computer | | | | |
| HTC5 | Super | NR | 70-120 | 40-85 | 45-65 |
| | Computer | | | | |
| HTC6 | Super | NR | 70-120 | 40-85 | 45-65 |
| | Computer | | | | |
| HTC1 | Super | NR | 70-150 | 65-75 | 45-65 |
| | Computer | | | | |
| HTC2 | Super | NR | 70-150 | 65-75 | 45-65 |
| | Computer | | | | |
| HTC3 | Super | NR | 70-150 | 65-75 | 45-65 |
| | Computer | | | | |
| HTC4 | Super | NR | 70-150 | 65-75 | 45-65 |
| | Computer | | | | |
| HTC5 | Super | NR | 70-150 | 65-75 | 45-65 |
| | Computer | | | | |
| HTC6 | Super | NR | 70-150 | 65-75 | 45-65 |
| | Computer | | | | |
| HTC1 | Server | NR | NR | NR | NR |
| HTC2 | Server | NR | NR | NR | NR |
| HTC3 | Server | NR | NR | NR | NR |
| HTC4 | Server | NR | NR | NR | NR |
| HTC5 | Server | NR | NR | NR | NR |
| HTC6 | Server | NR | NR | NR | NR |
| HTC1 | Server | NR | NR | >40 | NR |
| HTC2 | Server | NR | NR | >40 | NR |
| HTC3 | Server | NR | NR | >40 | NR |
| HTC4 | Server | NR | NR | >40 | NR |
| HTC5 | Server | NR | NR | >40 | NR |
| HTC6 | Server | NR | NR | >40 | NR |
| HTC1 | Server | NR | NR | 40-85 | NR |
| HTC2 | Server | NR | NR | 40-85 | NR |
| HTC3 | Server | NR | NR | 40-85 | NR |
| HTC4 | Server | NR | NR | 40-85 | NR |
| HTC5 | Server | NR | NR | 40-85 | NR |
| HTC6 | Server | NR | NR | 40-85 | NR |
| HTC1 | Server | NR | NR | 65-75 | NR |
| HTC2 | Server | NR | NR | 65-75 | NR |
| HTC3 | Server | NR | NR | 65-75 | NR |
| HTC4 | Server | NR | NR | 65-75 | NR |
| HTC5 | Server | NR | NR | 65-75 | NR |
| HTC6 | Server | NR | NR | 65-75 | NR |
| HTC1 | Server | NR | NR | >40 | 45-65 |
| HTC2 | Server | NR | NR | >40 | 45-65 |
| HTC3 | Server | NR | NR | >40 | 45-65 |
| HTC4 | Server | NR | NR | >40 | 45-65 |
| HTC5 | Server | NR | NR | >40 | 45-65 |
| HTC6 | Server | NR | NR | >40 | 45-65 |
| HTC1 | Server | NR | NR | 40-85 | 45-65 |
| HTC2 | Server | NR | NR | 40-85 | 45-65 |
| HTC3 | Server | NR | NR | 40-85 | 45-65 |
| HTC4 | Server | NR | NR | 40-85 | 45-65 |
| HTC5 | Server | NR | NR | 40-85 | 45-65 |
| HTC6 | Server | NR | NR | 40-85 | 45-65 |
| HTC1 | Server | NR | NR | 65-75 | 45-65 |
| HTC2 | Server | NR | NR | 65-75 | 45-65 |
| HTC3 | Server | NR | NR | 65-75 | 45-65 |
| HTC4 | Server | NR | NR | 65-75 | 45-65 |
| HTC5 | Server | NR | NR | 65-75 | 45-65 |
| HTC6 | Server | NR | NR | 65-75 | 45-65 |
| HTC1 | Server | NR | 50-150 | NR | NR |
| HTC2 | Server | NR | 50-150 | NR | NR |
| HTC3 | Server | NR | 50-150 | NR | NR |
| HTC4 | Server | NR | 50-150 | NR | NR |
| HTC5 | Server | NR | 50-150 | NR | NR |
| HTC6 | Server | NR | 50-150 | NR | NR |
| HTC1 | Server | NR | 50-150 | >40 | NR |
| HTC2 | Server | NR | 50-150 | >40 | NR |
| HTC3 | Server | NR | 50-150 | >40 | NR |
| HTC4 | Server | NR | 50-150 | >40 | NR |
| HTC5 | Server | NR | 50-150 | >40 | NR |
| HTC6 | Server | NR | 50-150 | >40 | NR |
| HTC1 | Server | NR | 50-150 | 40-85 | NR |
| HTC2 | Server | NR | 50-150 | 40-85 | NR |
| HTC3 | Server | NR | 50-150 | 40-85 | NR |
| HTC4 | Server | NR | 50-150 | 40-85 | NR |
| HTC5 | Server | NR | 50-150 | 40-85 | NR |
| HTC6 | Server | NR | 50-150 | 40-85 | NR |
| HTC1 | Server | NR | 50-150 | 65-75 | NR |
| HTC2 | Server | NR | 50-150 | 65-75 | NR |
| HTC3 | Server | NR | 50-150 | 65-75 | NR |
| HTC4 | Server | NR | 50-150 | 65-75 | NR |
| HTC5 | Server | NR | 50-150 | 65-75 | NR |
| HTC6 | Server | NR | 50-150 | 65-75 | NR |
| HTC1 | Server | NR | 50-150 | >40 | 45-65 |
| HTC2 | Server | NR | 50-150 | >40 | 45-65 |
| HTC3 | Server | NR | 50-150 | >40 | 45-65 |
| HTC4 | Server | NR | 50-150 | >40 | 45-65 |
| HTC5 | Server | NR | 50-150 | >40 | 45-65 |
| HTC6 | Server | NR | 50-150 | >40 | 45-65 |
| HTC1 | Server | NR | 50-150 | 40-85 | 45-65 |
| HTC2 | Server | NR | 50-150 | 40-85 | 45-65 |
| HTC3 | Server | NR | 50-150 | 40-85 | 45-65 |
| HTC4 | Server | NR | 50-150 | 40-85 | 45-65 |
| HTC5 | Server | NR | 50-150 | 40-85 | 45-65 |
| HTC6 | Server | NR | 50-150 | 40-85 | 45-65 |
| HTC1 | Server | NR | 50-150 | 65-75 | 45-65 |
| HTC2 | Server | NR | 50-150 | 65-75 | 45-65 |
| HTC3 | Server | NR | 50-150 | 65-75 | 45-65 |
| HTC4 | Server | NR | 50-150 | 65-75 | 45-65 |
| HTC5 | Server | NR | 50-150 | 65-75 | 45-65 |
| HTC6 | Server | NR | 50-150 | 65-75 | 45-65 |
| HTC1 | Server | NR | 70-120 | NR | NR |
| HTC2 | Server | NR | 70-120 | NR | NR |
| HTC3 | Server | NR | 70-120 | NR | NR |
| HTC4 | Server | NR | 70-120 | NR | NR |
| HTC5 | Server | NR | 70-120 | NR | NR |
| HTC6 | Server | NR | 70-120 | NR | NR |
| HTC1 | Server | NR | 70-120 | >40 | NR |
| HTC2 | Server | NR | 70-120 | >40 | NR |
| HTC3 | Server | NR | 70-120 | >40 | NR |
| HTC4 | Server | NR | 70-120 | >40 | NR |
| HTC5 | Server | NR | 70-120 | >40 | NR |
| HTC6 | Server | NR | 70-120 | >40 | NR |
| HTC1 | Server | NR | 70-120 | 40-85 | NR |
| HTC2 | Server | NR | 70-120 | 40-85 | NR |
| HTC3 | Server | NR | 70-120 | 40-85 | NR |
| HTC4 | Server | NR | 70-120 | 40-85 | NR |
| HTC5 | Server | NR | 70-120 | 40-85 | NR |
| HTC6 | Server | NR | 70-120 | 40-85 | NR |
| HTC1 | Server | NR | 70-120 | 65-75 | NR |
| HTC2 | Server | NR | 70-120 | 65-75 | NR |
| HTC3 | Server | NR | 70-120 | 65-75 | NR |
| HTC4 | Server | NR | 70-120 | 65-75 | NR |
| HTC5 | Server | NR | 70-120 | 65-75 | NR |
| HTC6 | Server | NR | 70-120 | 65-75 | NR |
| HTC1 | Server | NR | 70-120 | >40 | 45-65 |
| HTC2 | Server | NR | 70-120 | >40 | 45-65 |
| HTC3 | Server | NR | 70-120 | >40 | 45-65 |
| HTC4 | Server | NR | 70-120 | >40 | 45-65 |
| HTC5 | Server | NR | 70-120 | >40 | 45-65 |
| HTC6 | Server | NR | 70-120 | >40 | 45-65 |
| HTC1 | Server | NR | 70-120 | 40-85 | 45-65 |
| HTC2 | Server | NR | 70-120 | 40-85 | 45-65 |
| HTC3 | Server | NR | 70-120 | 40-85 | 45-65 |
| HTC4 | Server | NR | 70-120 | 40-85 | 45-65 |
| HTC5 | Server | NR | 70-120 | 40-85 | 45-65 |
| HTC6 | Server | NR | 70-120 | 40-85 | 45-65 |
| HTC1 | Server | NR | 70-120 | 65-75 | 45-65 |
| HTC2 | Server | NR | 70-120 | 65-75 | 45-65 |
| HTC3 | Server | NR | 70-120 | 65-75 | 45-65 |
| HTC4 | Server | NR | 70-120 | 65-75 | 45-65 |
| HTC5 | Server | NR | 70-120 | 65-75 | 45-65 |
| HTC6 | Server | NR | 70-120 | 65-75 | 45-65 |
| HTC1 | Server | data | NR | NR | NR |
| | | center | | | |
| HTC2 | Server | data | NR | NR | NR |
| | | center | | | |
| HTC3 | Server | data | NR | NR | NR |
| | | center | | | |
| HTC4 | Server | data | NR | NR | NR |
| | | center | | | |
| HTC5 | Server | data | NR | NR | NR |
| | | center | | | |
| HTC6 | Server | data | NR | NR | NR |
| | | center | | | |
| HTC1 | Server | data | NR | >40 | NR |
| | | center | | | |
| HTC2 | Server | data | NR | >40 | NR |
| | | center | | | |
| HTC3 | Server | data | NR | >40 | NR |
| | | center | | | |
| HTC4 | Server | data | NR | >40 | NR |
| | | center | | | |
| HTC5 | Server | data | NR | >40 | NR |
| | | center | | | |
| HTC6 | Server | data | NR | >40 | NR |
| | | center | | | |
| HTC1 | Server | data | NR | 40-85 | NR |
| | | center | | | |
| HTC2 | Server | data | NR | 40-85 | NR |
| | | center | | | |
| HTC3 | Server | data | NR | 40-85 | NR |
| | | center | | | |
| HTC4 | Server | data | NR | 40-85 | NR |
| | | center | | | |
| HTC5 | Server | data | NR | 40-85 | NR |
| | | center | | | |
| HTC6 | Server | data | NR | 40-85 | NR |
| | | center | | | |
| HTC1 | Server | data | NR | 65-75 | NR |
| | | center | | | |
| HTC2 | Server | data | NR | 65-75 | NR |
| | | center | | | |
| HTC3 | Server | data | NR | 65-75 | NR |
| | | center | | | |
| HTC4 | Server | data | NR | 65-75 | NR |
| | | center | | | |
| HTC5 | Server | data | NR | 65-75 | NR |
| | | center | | | |
| HTC6 | Server | data | NR | 65-75 | NR |
| | | center | | | |
| | Server | data | NR | 65-75 | NR |
| | | center | | | |
| HTC1 | Server | data | NR | 65-75 | NR |
| | | center | | | |
| HTC2 | Server | data | NR | 65-75 | NR |
| | | center | | | |
| HTC3 | Server | data | NR | 65-75 | NR |
| | | center | | | |
| HTC4 | Server | data | NR | 65-75 | NR |
| | | center | | | |
| HTC5 | Server | data | NR | 65-75 | NR |
| | | center | | | |
| HTC6 | Server | data | NR | 65-75 | NR |
| | | center | | | |
| HTC1 | Server | data | NR | 40-85 | 45-65 |
| | | center | | | |
| HTC2 | Server | data | NR | 40-85 | 45-65 |
| | | center | | | |
| HTC3 | Server | data | NR | 40-85 | 45-65 |
| | | center | | | |
| HTC4 | Server | data | NR | 40-85 | 45-65 |
| | | center | | | |
| HTC5 | Server | data | NR | 40-85 | 45-65 |
| | | center | | | |
| HTC6 | Server | data | NR | 40-85 | 45-65 |
| | | center | | | |
| HTC1 | Server | data | NR | 65-75 | 45-65 |
| | | center | | | |
| HTC2 | Server | data | NR | 65-75 | 45-65 |
| | | center | | | |
| HTC3 | Server | data | NR | 65-75 | 45-65 |
| | | center | | | |
| HTC4 | Server | data | NR | 65-75 | 45-65 |
| | | center | | | |
| HTC5 | Server | data | NR | 65-75 | 45-65 |
| | | center | | | |
| HTC6 | Server | data | NR | 65-75 | 45-65 |
| | | center | | | |
| HTC1 | Server | data | 50-150 | NR | NR |
| | | center | | | |
| HTC2 | Server | data | 50-150 | NR | NR |
| | | center | | | |
| HTC3 | Server | data | 50-150 | NR | NR |
| | | center | | | |
| HTC4 | Server | data | 50-150 | NR | NR |
| | | center | | | |
| HTC5 | Server | data | 50-150 | NR | NR |
| | | center | | | |
| HTC6 | Server | data | 50-150 | NR | NR |
| | | center | | | |
| HTC1 | Server | data | 50-150 | >40 | NR |
| | | center | | | |
| HTC2 | Server | data | 50-150 | >40 | NR |
| | | center | | | |
| HTC3 | Server | data | 50-150 | >40 | NR |
| | | center | | | |
| HTC4 | Server | data | 50-150 | >40 | NR |
| | | center | | | |
| HTC5 | Server | data | 50-150 | >40 | NR |
| | | center | | | |
| HTC6 | Server | data | 50-150 | >40 | NR |
| | | center | | | |
| HTC1 | Server | data | 50-150 | 40-85 | NR |
| | | center | | | |
| HTC2 | Server | data | 50-150 | 40-85 | NR |
| | | center | | | |
| HTC3 | Server | data | 50-150 | 40-85 | NR |
| | | center | | | |
| HTC4 | Server | data | 50-150 | 40-85 | NR |
| | | center | | | |
| HTC5 | Server | data | 50-150 | 40-85 | NR |
| | | center | | | |
| HTC6 | Server | data | 50-150 | 40-85 | NR |
| | | center | | | |
| HTC1 | Server | data | 50-150 | 65-75 | NR |
| | | center | | | |
| HTC2 | Server | data | 50-150 | 40-85 | NR |
| | | center | | | |
| HTC3 | Server | data | 50-150 | 40-85 | NR |
| | | center | | | |
| HTC4 | Server | data | 50-150 | 40-85 | NR |
| | | center | | | |
| HTC5 | Server | data | 50-150 | 40-85 | NR |
| | | center | | | |
| HTC6 | Server | data | 50-150 | 40-85 | NR |
| | | center | | | |
| HTC1 | Server | data | 50-150 | >40 | 45-65 |
| | | center | | | |
| HTC2 | Server | data | 50-150 | >40 | 45-65 |
| | | center | | | |
| HTC3 | Server | data | 50-150 | >40 | 45-65 |
| | | center | | | |
| HTC4 | Server | data | 50-150 | >40 | 45-65 |
| | | center | | | |
| HTC5 | Server | data | 50-150 | >40 | 45-65 |
| | | center | | | |
| HTC6 | Server | data | 50-150 | >40 | 45-65 |
| | | center | | | |
| HTC1 | Server | data | 50-150 | >45 | 45-65 |
| | | center | | | |
| HTC2 | Server | data | 50-150 | >45 | 45-65 |
| | | center | | | |
| HTC3 | Server | data | 50-150 | >45 | 45-65 |
| | | center | | | |
| HTC4 | Server | data | 50-150 | >45 | 45-65 |
| | | center | | | |
| HTC5 | Server | data | 50-150 | >45 | 45-65 |
| | | center | | | |
| HTC6 | Server | data | 50-150 | >45 | 45-65 |
| | | center | | | |
| HTC1 | Server | data | 50-150 | 40-85 | 45-65 |
| | | center | | | |
| HTC2 | Server | data | 50-150 | 40-85 | 45-65 |
| | | center | | | |
| HTC3 | Server | data | 50-150 | 40-85 | 45-65 |
| | | center | | | |
| HTC4 | Server | data | 50-150 | 40-85 | 45-65 |
| | | center | | | |
| HTC5 | Server | data | 50-150 | 40-85 | 45-65 |
| | | center | | | |
| HTC6 | Server | data | 50-150 | 40-85 | 45-65 |
| | | center | | | |
| HTC1 | Server | data | 50-150 | 65-75 | 45-65 |
| | | center | | | |
| HTC2 | Server | data | 50-150 | 65-75 | 45-65 |
| | | center | | | |
| HTC3 | Server | data | 50-150 | 65-75 | 45-65 |
| | | center | | | |
| HTC4 | Server | data | 50-150 | 65-75 | 45-65 |
| | | center | | | |
| HTC5 | Server | data | 50-150 | 65-75 | 45-65 |
| | | center | | | |
| HTC6 | Server | data | 50-150 | 65-75 | 45-65 |
| | | center | | | |
| HTC1 | Server | data | 70-120 | NR | NR |
| | | center | | | |
| HTC2 | Server | data | 70-120 | NR | NR |
| | | center | | | |
| HTC3 | Server | data | 70-120 | NR | NR |
| | | center | | | |
| HTC4 | Server | data | 70-120 | NR | NR |
| | | center | | | |
| HTC5 | Server | data | 70-120 | NR | NR |
| | | center | | | |
| HTC6 | Server | data | 70-120 | NR | NR |
| | | center | | | |
| HTC1 | Server | data | 70-120 | >40 | NR |
| | | center | | | |
| HTC2 | Server | data | 70-120 | >40 | NR |
| | | center | | | |
| HTC3 | Server | data | 70-120 | >40 | NR |
| | | center | | | |
| HTC4 | Server | data | 70-120 | >40 | NR |
| | | center | | | |
| HTC5 | Server | data | 70-120 | >40 | NR |
| | | center | | | |
| HTC6 | Server | data | 70-120 | >40 | NR |
| | | center | | | |
| HTC1 | Server | data | 70-120 | >45 | NR |
| | | center | | | |
| HTC2 | Server | data | 70-120 | >45 | NR |
| | | center | | | |
| HTC3 | Server | data | 70-120 | >45 | NR |
| | | center | | | |
| HTC4 | Server | data | 70-120 | >45 | NR |
| | | center | | | |
| HTC5 | Server | data | 70-120 | >45 | NR |
| | | center | | | |
| HTC6 | Server | data | 70-120 | >45 | NR |
| | | center | | | |
| HTC1 | Server | data | 70-120 | 40-85 | NR |
| | | center | | | |
| HTC2 | Server | data | 70-120 | 40-85 | NR |
| | | center | | | |
| HTC3 | Server | data | 70-120 | 40-85 | NR |
| | | center | | | |
| HTC4 | Server | data | 70-120 | 40-85 | NR |
| | | center | | | |
| HTC5 | Server | data | 70-120 | 40-85 | NR |
| | | center | | | |
| HTC6 | Server | data | 70-120 | 40-85 | NR |
| | | center | | | |
| HTC1 | Server | data | 70-120 | 65-75 | NR |
| | | center | | | |
| HTC2 | Server | data | 70-120 | 65-75 | NR |
| | | center | | | |
| HTC3 | Server | data | 70-120 | 65-75 | NR |
| | | center | | | |
| HTC4 | Server | data | 70-120 | 65-75 | NR |
| | | center | | | |
| HTC5 | Server | data | 70-120 | 65-75 | NR |
| | | center | | | |
| HTC6 | Server | data | 70-120 | 65-75 | NR |
| | | center | | | |
| HTC1 | Server | data | 70-120 | >40 | 45-65 |
| | | center | | | |
| HTC2 | Server | data | 70-120 | >40 | 45-65 |
| | | center | | | |
| HTC3 | Server | data | 70-120 | >40 | 45-65 |
| | | center | | | |
| HTC4 | Server | data | 70-120 | >40 | 45-65 |
| | | center | | | |
| HTC5 | Server | data | 70-120 | >40 | 45-65 |
| | | center | | | |
| HTC6 | Server | data | 70-120 | >40 | 45-65 |
| | | center | | | |
| HTC1 | Server | data | 70-120 | >45 | 45-65 |
| | | center | | | |
| HTC2 | Server | data | 70-120 | >45 | 45-65 |
| | | center | | | |
| HTC3 | Server | data | 70-120 | >45 | 45-65 |
| | | center | | | |
| HTC4 | Server | data | 70-120 | >45 | 45-65 |
| | | center | | | |
| HTC5 | Server | data | 70-120 | >45 | 45-65 |
| | | center | | | |
| HTC6 | Server | data | 70-120 | >45 | 45-65 |
| | | center | | | |
| HTC1 | Server | data | 70-120 | 40-85 | 45-65 |
| | | center | | | |
| HTC2 | Server | data | 70-120 | 40-85 | 45-65 |
| | | center | | | |
| HTC3 | Server | data | 70-120 | 40-85 | 45-65 |
| | | center | | | |
| HTC4 | Server | data | 70-120 | 40-85 | 45-65 |
| | | center | | | |
| HTC5 | Server | data | 70-120 | 40-85 | 45-65 |
| | | center | | | |
| HTC6 | Server | data | 70-120 | 40-85 | 45-65 |
| | | center | | | |
| HTC1 | Server | data | 70-120 | 65-75 | 45-65 |
| | | center | | | |
| HTC2 | Server | data | 70-120 | 65-75 | 45-65 |
| | | center | | | |
| HTC3 | Server | data | 70-120 | 65-75 | 45-65 |
| | | center | | | |
| HTC4 | Server | data | 70-120 | 65-75 | 45-65 |
| | | center | | | |
| HTC5 | Server | data | 70-120 | 65-75 | 45-65 |
| | | center | | | |
| HTC6 | Server | data | 70-120 | 65-75 | 45-65 |
| | | center | | | |

*Reference to an HTC number in this table is intended to identify each of the HTC numbers which have the indicated number as root. Thus, for example, reference in this table to HTC1 is intended to convey that all HTCs in Table 3 with a root of HTC1 are included, such as HTC1-1A1, etc.

Heat Pipe Cooling and Heating

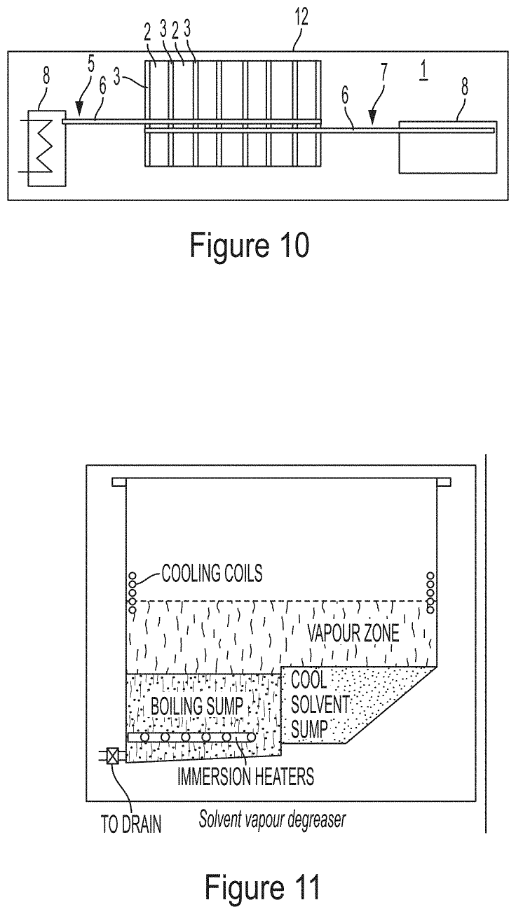

An example of the present heat transfer methods, including Heat Transfer Methods 1 and 2 and Thermal Management Methods 1-2, using a heat pipe is now described with respect to FIG. 10, which is a specific example of a heat pipe in an energy storage assembly 1 according to one exemplary embodiment of the invention. The energy storage assembly 1 may be part of a motor vehicle 12, in particular of a hybrid or electric vehicle, and is provided to supply electric power to electric consumers, like e.g. an electrical drive unit (not shown), on the motor vehicle side. The energy storage assembly 1 includes a plurality of electrical energy stores. 2. The electrical energy stores 2 are electrically connected via an electric connection element (not shown), in particular in the form of a conductive rail or conductor rail (“bus bar”), i.e., connected in series or in parallel. The electric connection element contacts hereby corresponding electrical connectors (not shown) arranged on respective exposed outer wall sections of corresponding energy storage housings (not shown) of the energy stores 2 in parallel alignment arranged adjacent to one another to thereby form an energy store stack (“stack”). Plate-shaped spacer elements 3 are respectively arranged between the energy stores 2 to separate them and at the same time thermally conductive properties. The spacer elements 3 thus provide, on one hand, spacing between immediately adjacent energy stores 2 so that immediately adjacent energy stores 2 do not contact each other electrically or mechanically. On the other hand, the spacer elements 3 act as a result of their thermally conductive properties as heat conductors for the purpose of cooling the energy stores 2 or the energy storage assembly 1 by dissipating heat, particularly from the contacting energy stores 2, or, for the purpose of heating the energy stores 2 or energy storage assembly 1 by supplying heat, particularly to the contacting energy stores 2. A heat pipe 4 of a first heat pipe assembly 5 and of a heat pipe 6 of a second heat pipe assembly 7 are provide. The heat pipes 4, 6 thus extend along this side surface of the energy store stack and are thermally coupled to the spacer elements 3, respectively. The spacer elements 3 thus form a thermal bridge between the heat pipes 4 of the first heat pipe assembly 5 and the heat pipes 6 of the second heat pipe assembly 7, on one hand, and the energy stores 2, on the other hand. The respective heat pipes 4 of the first heat pipe assembly 5 are arranged and aligned such as to be thermally coupled with respective evaporation zones, in which a contained thermal management fluid of the present invention, including particularly each of Compositions 1-6, and each of the heat transfer compositions in Table 3 above, namely HTC1-HTC6, can be evaporated, to the spacer elements 3. Heat (evaporation heat) required for the evaporation of the present TMF is thus removed from the spacer elements 3 or via the spacer elements 3 from the energy stores 2. The energy stores 2, including the energy storage assembly 1, can thus be cooled via the heat pipes 4 of the first heat pipe assembly 5. Also, the respective condensation zones of the heat pipes 4 of the first heat pipe assembly 5, in which condensation zones a contained gaseous thermal management fluid of the present invention, including particularly each of Compositions 1-6, and each of the heat transfer compositions in Table 3 above, namely HTC1-HTC6, can be condensed, are thermally coupled with a heat sink 8 in the form of a motor-vehicle-side heat exchanger. Heat (condensation heat) generated during condensation of the present TMF can thus be transferred to the heat sink 8. The heat exchanger can be part of the energy storage assembly 1, i.e., belong to or associated with the energy storage assembly 1. The respective heat pipes 6 of the second heat pipe assembly 7 are arranged and aligned such as to be thermally coupled with their respective condensation zones, in which the contained gaseous TMF of the present invention can be condensed, to the spacer elements 3. Heat (condensation heat) can thus be transferred during condensation of the present TMF to the spacer elements 3 or via the spacer elements 3 to the energy stores 2. Thus, the energy stores 2 and the energy storage assembly 1, can be heated via the heat pipes 6 of the second heat pipe assembly 7. Also, the respective evaporation zones of the heat pipes 6 of the second heat pipe assembly 7, in which evaporation zones a contained TMF of the present invention can be evaporated, are thermally coupled with a heat source 9 in the form of a functional component, i.e., e.g., a charger or a control device or a control electronics, associated to the energy storage assembly 1. Heat (evaporation heat) required for the evaporation of the TMF can thus be removed from the heat source 9. Thus, the functional component can be cooled via the heat pipes 6 of the second heat pipe assembly 7. The two heat pipe assemblies 5, 7, and their associated heat pipes 4, 6 enables implementation of a temperature control device for controlling the temperature, i.e., for heating or cooling, of the energy stores 2 of the energy storage assembly 1. The heat pipes useful according to the present invention include both gravity return heat pipe, capillary return heat pipe and gravity/capillary return heat pipes.

### Uses and Methods of Refrigerant and Heat Transfer Composition

The invention also provides a heat transfer system comprising a refrigerant or a heat transfer composition of the invention. It will be appreciated that the heat transfer systems described herein may be vapor compression systems having an evaporator, a condenser and a compressor in fluid communication.

The refrigerant or heat transfer composition of the invention, including each of Compositions 1-6, and each of the heat transfer compositions in Table 3 above, namely HTC1-HTC6, may be used as a secondary fluid.

It will be appreciated that the refrigerant or heat transfer composition of the invention, including each of Compositions 1-6, and each of the heat transfer compositions in Table 3 above, namely HTC1-HTC6, may be used in a variety of different heat transfer applications.

Organic Rankine Cycle

As discussed above, when a heat transfer fluid of the present invention, including each of Compositions 1-6, and each of the heat transfer compositions in Table 3 above, namely HTC1-HTC6, is used in an Organic Rankine cycle, it is referred to as a working fluid. The working fluid therefore corresponds to the heat transfer fluid as discussed in this application. All preferred features of the heat transfer fluid apply to the working fluid as described herein.

Rankine cycle systems are known to be a simple and reliable means to convert heat energy into mechanical energy in the form of shaft power. In industrial settings, it may be possible to use flammable working fluids such as toluene and pentane, particularly when the industrial setting has large quantities of flammables already on site in processes or storage. However, for instances where the risk associated with use of a flammable and/or toxic working fluid is not acceptable, such as power generation in populous areas or near buildings, it is necessary or at least highly desirable to use non-flammable and/or non-toxic refrigerants as the working fluid. There is also a drive in the industry for these materials to be environmentally acceptable in terms of GWP.

The process for recovering waste heat in an Organic Rankine cycle according to the present invention preferably involves pumping liquid-phase working fluid of the present invention, including each of Compositions 1-6, and each of the heat transfer compositions in Table 3 above, namely HTC1-HTC6, through a boiler where an external (waste) heat source, such as a process stream, heats the working fluid causing it to evaporate into a saturated or superheated vapor. This vapor is expanded through a turbine wherein the waste heat energy is converted into mechanical energy. Subsequently, the vapor phase working fluid is condensed to a liquid and pumped back to the boiler in order to repeat the heat extraction cycle.

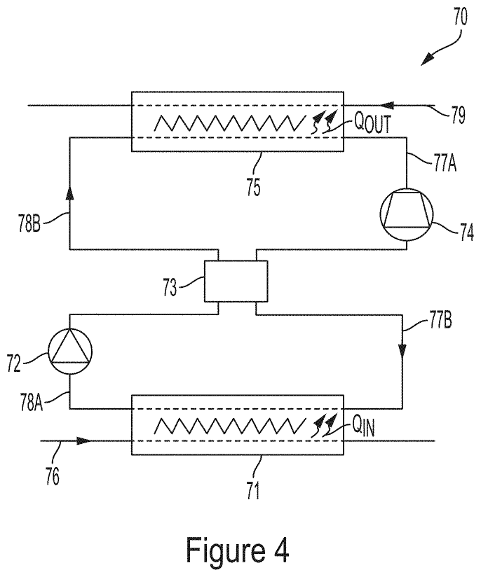

Referring to FIG. 4, in an exemplary organic Rankine cycle system 70, working fluid of the present invention, including each of Compositions 1-6, and each of the heat transfer compositions in Table 3 above, namely HTC1-HTC6, is circulated between an evaporator 71 and a condenser 75, with a pump 72 and an expansion device 74 functionally disposed therebetween. In the illustrated embodiment, an external flow of fluid is directed to evaporator 71 via external warm conduit 76. External warm conduit 76 may carry fluid from a warm heat source, such as a waste heat source from industrial processes (e.g., power generation), flue gases, exhaust gases, geothermal sources, etc.

Evaporator 71 is preferably configured as a heat exchanger which may include, e.g., a series of thermally connected, but fluidly isolated, tubes carrying fluid from warm conduit 76 and fluid from working fluid conduit 77B respectively. Thus, evaporator 71 facilitates the transfer of heat QIN from the warm fluid arriving from external warm conduit 76 to the relatively cooler (e.g., “cold”) working fluid arriving from expansion device 74 via working fluid conduit 77B.

The working fluid of the present invention, including each of Compositions 1-6, thus exits from evaporator 71, having been warmed by the absorption of heat QIN, and then travels through working fluid conduit 78A to pump 72. Pump 72 pressurizes the working fluid, thereby further warming the fluid through external energy inputs (e.g., electricity). The resulting “hot” fluid passes to an input of condenser 75 via conduit 78B, optionally via a regenerator 73 as described below.

Condenser 75 is configured as a heat exchanger similar to evaporator 71, and may include, e.g., a series of thermally connected, but fluidly isolated, tubes carrying fluid from cool conduit 79 and fluid from working fluid conduit 78B respectively. Condenser 75 facilitates the transfer of heat Q_{OUT} to the cool fluid arriving from external cool conduit 79 to the relatively warmer (e.g., “hot”) working fluid of the present invention, including each of Compositions 1-17 and 18A, arriving from pump 72 via working fluid conduit 78B.

The working fluid of the present invention, including each of Compositions 1-6, exiting from condenser 75, having thus been cooled by the loss of heat Q_{OUT}, then travels through working fluid conduit 77A to expansion device 74. Expansion device 74 allows the working fluid to expand, thereby further cooling the fluid. At this stage, the fluid of the present invention, including each of Compositions 1-6, and each of the heat transfer compositions in Table 3 above, namely HTC1-HTC6, may perform work, e.g., by driving a turbine. The resulting “cold” fluid passes to an input of evaporator 71 via conduit 77B, optionally via a regenerator 73 as described below, and the cycle begins anew.

Thus, working fluid conduits 77A, 77B, 78A and 78B define a closed loop such that the working fluid contained therein may be reused indefinitely, or until routing maintenance is required.

In the illustrated embodiment, regenerator 73 may be functionally disposed between evaporator 71 and condenser 75. Regenerator 73 allows the “hot” working fluid of the present invention, including each of Compositions 1-6, and each of the heat transfer compositions in Table 3 above, namely HTC1-HTC6, exiting from pump 72 and the “cold” working fluid issued from expansion device 74 to exchange some heat, potentially with a time lag between deposit of heat from the hot working fluid and release of that heat to the cold working fluid. In some applications, this can increase the overall thermal efficiency of Rankine cycle system 70.

Therefore, the invention relates to an organic Rankine cycle comprising a working fluid of the present invention, including each of Compositions 1-6, and each of the heat transfer compositions in Table 3 above, namely HTC1-HTC6.

The invention further relates to the use of a working fluid of the invention, including each of Compositions 1-17 and 18A, in an Organic Rankine Cycle.

The invention also provides a process for converting thermal energy to mechanical energy in a Rankine cycle, the method comprising the steps of i) vaporizing a working fluid of the invention, including each of Compositions 1-6, and each of the heat transfer compositions in Table 3 above, namely HTC1-HTC6, with a heat source and expanding the resulting vapor, then ii) cooling the working fluid with a heat sink to condense the vapor, wherein the working fluid is a refrigerant or heat transfer composition of the invention, including each of Compositions 1-6, and each of the heat transfer compositions in Table 3 above, namely HTC1-HTC6.

The mechanical work may be transmitted to an electrical device such as a generator to produce electrical power.

The heat source maybe provided by, for example, a thermal energy source selected from industrial waste heat, solar energy, geothermal hot water, low pressure steam, distributed power generation equipment utilizing fuel cells, prime movers, or an internal combustion engine. The low-pressure steam is preferably a low pressure geothermal steam or is provided by a fossil fuel powered electrical generating power plant.

The heat source is preferably provided by a thermal energy source selected from industrial waste heat, or an internal combustion engine.

It will be appreciated that the heat source temperatures can vary widely, for example from about 90° C. to >800° C., and can be dependent upon a myriad of factors including geography, time of year, etc. for certain combustion gases and some fuel cells.

Systems based on sources such as waste water or low pressure steam from, e.g., a plastics manufacturing plants and/or from chemical or other industrial plant, petroleum refinery, and related word forms, as well as geothermal sources, may have source temperatures that are at or below about 175° C. or at or below about 100° C., and in some cases as low as about 90° C. or even as low as about 80° C. Gaseous sources of heat such as exhaust gas from combustion process or from any heat source where subsequent treatments to remove particulates and/or corrosive species result in low temperatures may also have source temperatures that are at or below 200° C., at or below about 175° C., at or below about 130° C., at or below about 120° C., at or below about 100° C., at or below about 100° C., and in some cases as low as about 90° C. or even as low as about 80° C.

However, it is preferred in some applications that the heat source has a temperature of at least about 200° C., for example of from about 200° C. to about 400° C.

In an alternative preferred embodiment, the heat source has a temperature of from 400 to 800° C., more preferably 400 to 600° C.

Heat Pump

As discussed above, when a heat transfer fluid of the present invention, including each of Compositions 1-6, and each of the heat transfer compositions in Table 3 above, namely HTC1-HTC6, used in a heat pump, it is referred to as a refrigerant. The refrigerant therefore corresponds to the heat transfer fluid as discussed in this application. All preferred features of the heat transfer fluid as described apply to the refrigerant as described herein.

The refrigerant or heat transfer composition of the invention, including each of Compositions 1-6, and each of the heat transfer compositions in Table 3 above, namely HTC1-HTC6, may be used in a high temperature heat pump system.

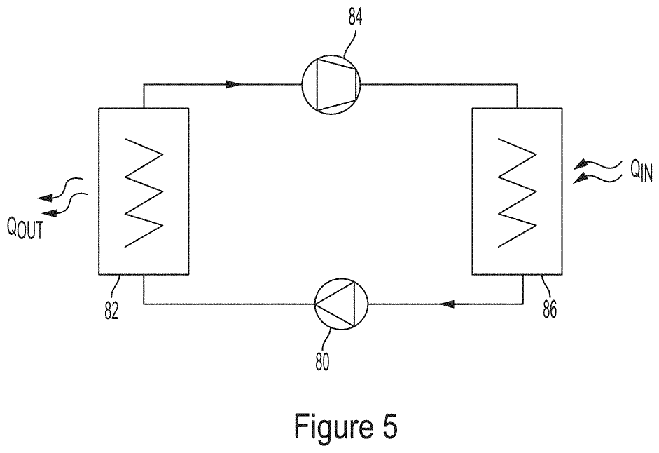

Referring to FIG. 5, in one exemplary heat pump system, compressor 80, such as a rotary, piston, screw, or scroll compressor, compresses a refrigerant of the present invention, including each of Compositions 1-6, and each of the heat transfer compositions in Table 3 above, namely HTC1-HTC6,

which is conveyed to a condenser 82 to release heat Q_{OUT} to a first location, followed by passing the refrigerant through an expansion device 84 to lower the refrigerant pressure, followed by passing the refrigerant through an evaporator 86 to absorb heat QIN from a second location. The refrigerant is then conveyed back to the compressor 80 for compression.

The present invention provides a method of heating a fluid or body using a high temperature heat pump, said method comprising the steps of (a) condensing a refrigerant composition of the present invention, including each of Compositions 1-6, and each of the heat transfer compositions in Table 3 above, namely HTC1-HTC6, in the vicinity of the fluid of body or be heated, and (b) evaporating said refrigerant.

Examples of high temperature heat pumps include a heat pump tumble dryer or an industrial heat pump. It will be appreciated the heat pump may comprise a suction line/liquid line heat exchanger (SL-LL HX). By “high temperature heat pump”, it is meant a heat pump that is able to generate temperatures of at least about 80° C., preferably at least about 90° C., preferably at least about 100° C., more preferably at least about 110° C.

Secondary Loop System

As discussed above, when the heat transfer fluid of the present invention, including each of Compositions 1-6, and each of the heat transfer compositions in Table 3 above, namely HTC1-HTC6, is used in a secondary loop system, it is referred to as a refrigerant.

The refrigerant of the present invention, including each of Compositions 1-6, and each of the heat transfer compositions in Table 3 above, namely HTC1-HTC6, may be used as secondary refrigerant fluid in a secondary loop system.

A secondary loop system contains a primary vapor compression system loop that uses a primary refrigerant and whose evaporator cools the secondary loop fluid. The secondary refrigerant fluid, including each of Compositions 1-6, and each of the heat transfer compositions in Table 3 above, namely HTC1-HTC6, then provides the necessary cooling for an application. The secondary refrigerant fluid should preferably be non-flammable and have low toxicity since the fluid in such a loop is potentially exposed to humans in the vicinity of the cooled space. In other words, the refrigerant or heat transfer composition of the present invention, including each of Compositions 1-6, and each of the heat transfer compositions in Table 3 above, namely HTC1-HTC6, may be used as a “secondary refrigerant fluid” in a secondary loop system.

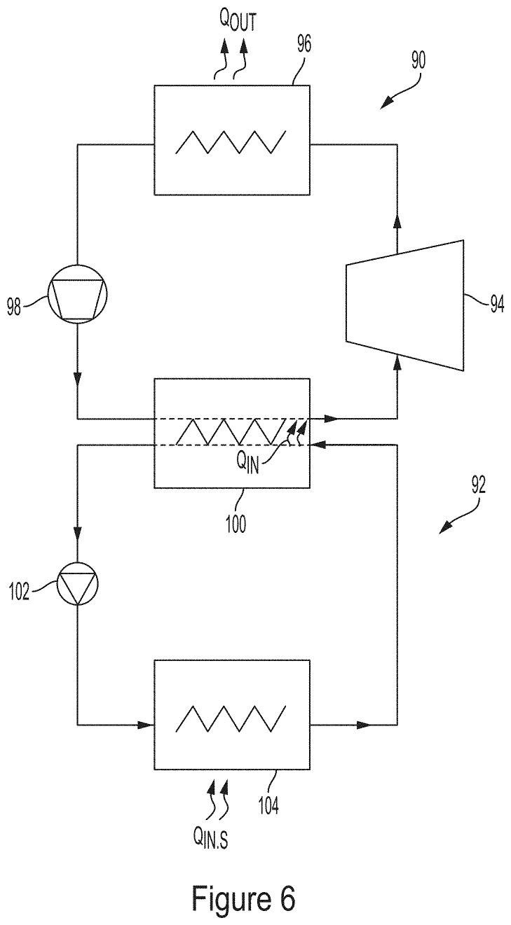

Referring to FIG. 6, one exemplary secondary loop system includes a primary loop 90 and a secondary loop 92. In primary loop 90, compressor 94, such as a rotary, piston, screw, or scroll compressor, compresses a primary refrigerant, which is conveyed to a condenser 96 to release heat Q_{OUT} to a first location, followed by passing the primary refrigerant through an expansion device 98 to lower the refrigerant pressure, followed by passing the primary refrigerant through a refrigerant/secondary fluid heat exchanger 100 to exchange heat QIN with a secondary fluid, including each of Compositions 1-6, and each of the heat transfer compositions in Table 3 above, namely HTC1-HTC6, with the secondary fluid pumped through secondary loop 92 via a pump 102 to a secondary loop heat exchanger 104 to exchange heat with a further location, for example to absorb heat QIN-s to providing cooling to the further location.

The primary fluid used in the primary loop (vapor compression cycle, external/outdoors part of the loop) may be selected from but not limited to HFO-1234ze(E), HFO-1234yf, propane, R455A, R32, R466A, R44B, R290, R717, R452B, R448A, and R449A, preferably HFO-1234ze(E), HFO-1234yf, or propane.

The secondary loop system may be used in refrigeration or air conditioning applications, that is, the secondary loop system may be a secondary loop refrigeration system or a secondary loop air conditioning system.

Examples of refrigeration systems which can include a secondary loop refrigeration system that include a secondary refrigerant of the present invention, including each of Compositions 1-6, and each of the heat transfer compositions in Table 3 above, namely HTC1-HTC6, include:
a low temperature refrigeration system,
a medium temperature refrigeration system,
a commercial refrigerator,
a commercial freezer,
an industrial freezer,
an industrial refrigerator and
a chiller.

Examples of air conditioning systems which can include a secondary loop air conditioning system which utilize a refrigerant of the present invention, including each of Compositions 1-6, and each of the heat transfer compositions in Table 3 above, namely HTC1-HTC6, include in mobile air conditioning systems or stationary air conditioning systems. Mobile air-conditioning systems including air conditioning of road vehicles such as automobiles, trucks and buses, as well as air conditioning of boats, and trains. For example, where a vehicle contains a battery or electric power source.

Examples of stationary air conditioning systems which can include a secondary loop air conditioning system which utilize a refrigerant of the present invention, including each of Compositions 1-6, and each of the heat transfer compositions in Table 3 above, namely HTC1-HTC6, include:
a chiller, particularly a positive displacement chiller, more particularly an air cooled or water-cooled direct expansion chiller, which is either modular or conventionally singularly packaged,
a residential air conditioning system, particularly a ducted split or a ductless split air conditioning system,
a residential heat pump,
a residential air to water heat pump/hydronic system,
an industrial air conditioning system,
a commercial air conditioning system, particularly a packaged rooftop unit and a variable refrigerant flow (VRF) system, and
a commercial air source, water source or ground source heat pump system.

A particularly preferred heat transfer system according to the present invention is an automotive air conditioning system comprising a vapour compression system (the primary loop) and a secondary loop air conditioning system, wherein the primary loop contains HFO-1234yf as the refrigerant and the second loop contains a refrigerant or heat transfer composition of the present invention, including each of Compositions 1-6, and each of the heat transfer compositions in Table 3 above, namely HTC1-HTC6. In particular, the secondary loop can be used to cool a component in the car engine, such as the battery.

It will be appreciated the secondary loop air conditioning or refrigeration system may comprise a suction line/liquid line heat exchanger (SL-LL HX).

The present heat transfer fluids, or heat transfer compositions which can include a secondary loop air conditioning system which utilize a refrigerant of the present invention, including each of Compositions 1-6, and each of the heat transfer compositions in Table 3 above, namely HTC1-HTC6, may be used as a replacement for existing fluids.

The invention includes a method of replacing an existing heat transfer fluid in a heat transfer system, said method comprising the steps of (a) removing at least a portion of said existing heat transfer fluid from said system, and subsequently (b) introducing into said system a heat transfer fluid of the invention. Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % or substantially all of said existing heat transfer fluid from said system prior to step (b).

The method may optionally comprise the step of flushing said system with a solvent after conducting step (a) and prior to conducting step (b).

For the purposes of this invention, the heat transfer fluid of the present invention, including each of Compositions 1-6, and each of the heat transfer compositions in Table 3 above, namely HTC1-HTC6, can be used to replace an existing fluid in an electronic device, in an Organic Rankine cycle, in a high temperature heat pump or in a secondary loop.

For example, the thermal management fluid of the invention, including each of Compositions 1-17 and 18A, may be used as a replacement for existing fluids such as HFC-4310mee, HFE-7100 and HFE-7200. Alternatively, the thermal management fluid, including each of Compositions 1-6, and each of the heat transfer compositions in Table 3 above, namely HTC1-HTC6, can be used to replace water and glycol. The replacement may be in existing systems, or in new systems which are designed to work with an existing fluid. Alternatively, the thermal management fluid, including each of Compositions 1-6, and each of the heat transfer compositions in Table 3 above, namely HTC1-HTC6, can be used in applications in which the existing refrigerant was previously used. Alternatively, the refrigerant of the present invention, including each of Compositions 1-6, and each of the heat transfer compositions in Table 3 above, namely HTC1-HTC6, may be used to retrofit an existing refrigerant in an existing system. Alternatively, the refrigerant of the present invention, including each of Compositions 1-6, and each of the heat transfer compositions in Table 3 above, namely HTC1-HTC6, may be used in new systems which are designed to work with an existing refrigerant.

The invention provides a method of replacing an existing refrigerant in a heat transfer system, said method comprising the steps of (a) removing at least a portion of said existing refrigerant from said system, and subsequently (b) introducing into said system a refrigerant of the invention of the present invention, including each of Compositions 1-6, and each of the heat transfer compositions in Table 3 above, namely HTC1-HTC6. The existing refrigerants may be selected, for example, from HFC-4310mee, HFE-7100 and HFE-7200.

Step (a) may involve removing at least about 5 wt. %, at least about 10 wt. %, at least about 15 wt. %, at least about 50 wt. % at least about 70 wt. %, at least about 90 wt. %, at least about 95 wt. %, at least about 99 wt. % or at least about 99.5 wt. % of said existing refrigerant from said system prior to step (b).

The method may optionally comprise the step of flushing said system with a solvent after conducting step (a) and prior to conducting step (b).

### Solvent and Cleaning Uses, Methods and Systems

The present invention provides solvating methods. Such methods include cleaning methods generally, etching methods, carrier solvent applications (for coating applications, lubricant deposition, silicone deposition, and other coatings, including in connection with coatings of medical devices heparin and PTFE for example).

With respect to cleaning methods, all such methods are included within the scope of the present invention. Preferred cleaning methods include vapor degreasing by contacting the article, device or component thereof with a composition of the present invention, including each of Compositions 1-6, and each of the working fluids in Table 2 above, namely WF1-WF6. A wide variety of contaminants can be removed from a wide variety of article, device and components. Examples of contaminants that can be removed using a composition of the present invention, including each of Compositions 1-6, and each of the working fluids in Table 2 above, namely WF1-WF6, include, for example, light oils, medium oils, fluorolubes, greases and silicones and waxes. Examples of article, device and components that can be cleaned using a composition of the present invention, including each of Compositions 1-6, and each of the working fluids in Table 2 above, namely WF1-WF6, include, for example electronic components (including silicon wafers, PCBs, semiconductor surfaces), precision parts (including aircraft parts and components) light oils, medium oils, fluorolubes, greases and silicones and waxes.

Preferred solvent vapor phase degreasing and defluxing methods of the present invention include immersing a soiled substrate or part (e.g., a printed circuit board or a fabricated metal, glass, ceramic, plastic, or elastomer part or composite) or a portion of a substrate or part into a boiling, non-flammable liquid in accordance with the present invention, including each of Compositions 1-6, and each of the working fluids in Table 2 above, namely WF1-WF6, followed by rinsing the part in a second tank or cleaning zone by immersion or distillate spray with a clean solvent which can also be any one of the compositions of the present invention. The parts are then dried by maintaining the cooled part in the condensing vapours until temperature has reached equilibrium.

Solvent cleaning of various types of parts generally occurs in batch, hoist-assisted batch, conveyor batch, or in-line type conveyor degreaser and defluxer equipment. Parts may also be cleaned in open top defluxing or degreasing equipment. In both types of equipment, the entrance and/or exit ends of the equipment can be in open communication with both the ambient environment and the solvent within the equipment. In order to minimize the loss of solvent from the equipment by either convection or diffusion, a common practice in the art is to use.

The compositions of the present invention comprise a solvent cleaning composition that includes any compound within Compositions 1-6, and each of the working fluids in Table 2 above, namely WF1-WF6, and co-solvent in amounts as indicated in the Table 5 below based on the total weight of the solvent components in the composition, with each amount being understood to be preceded by the word “about”:

TABLE 5

| Solvent Composition | Wt. % Compound of | Wt. % of Co-solvent |
|---|---|---|
| No. (SC No.) | the invention | component(s) |
| SC1 | 100 Compound 1 | 0 |
| | 1-99 Compound 1 | 1-99 |
| | 1-10 Compound 1 | 90-99 |
| | 30-50 Compound 1 | 50-70 |
| | 50-70 Compound 1 | 30-50 |
| | 70-90 Compound 1 | 10-30 |
| | 90-99 Compound 1 | 1-10 |
| SC1b | 100 Compound 1B | 0 |
| | 1-99 Compound 1B | 1-99 |
| | 1-10 Compound 1B | 90-99 |
| | 30-50 Compound 1B | 50-70 |
| | 50-70 Compound 1B | 30-50 |
| | 70-90 Compound 1B | 10-30 |
| | 90-99 Compound 1B | 1-10 |
| SC1c | 100 Compound 1C | 0 |
| | 1-99 Compound 1C | 1-99 |
| | 1-10 Compound 1C | 90-99 |
| | 30-50 Compound 1C | 50-70 |
| | 50-70 Compound 1C | 30-50 |
| | 70-90 Compound 1C | 10-30 |
| | 90-99 Compound 1C | 1-10 |
| SC1d | 100 Compound 1D | 0 |
| | 1-99 Compound 1D | 1-99 |
| | 1-10 Compound 1D | 90-99 |
| | 30-50 Compound 1D | 50-70 |
| | 50-70 Compound 1D | 30-50 |
| | 70-90 Compound 1D | 10-30 |
| | 90-99 Compound 1D | 1-10 |
| SC1e | 100 Compound 1E | 0 |
| | 1-99 Compound 1E | 1-99 |
| | 1-10 Compound 1E | 90-99 |
| | 30-50 Compound 1E | 50-70 |
| | 50-70 Compound 1E | 30-50 |
| | 70-90 Compound 1E | 10-30 |
| | 90-99 Compound 1E | 1-10 |
| SC1f | 100 Compound 1F | 0 |
| | 1-99 Compound 1F | 1-99 |
| | 1-10 Compound 1F | 90-99 |
| | 30-50 Compound 1F | 50-70 |
| | 50-70 Compound 1F | 30-50 |
| | 70-90 Compound 1F | 10-30 |
| | 90-99 Compound 1F | 1-10 |
| SC1g | 100 Compound 1G | 0 |
| | 1-99 Compound 1G | 1-99 |
| | 1-10 Compound 1G | 90-99 |
| | 30-50 Compound 1G | 50-70 |
| | 50-70 Compound 1G | 30-50 |
| | 70-90 Compound 1G | 10-30 |
| | 90-99 Compound 1G | 1-10 |
| SC1h | 100 Compound 1H | 0 |
| | 1-99 Compound 1H | 1-99 |
| | 1-10 Compound 1Hh | 90-99 |
| | 30-50 Compound 1H | 50-70 |
| | 50-70 Compound 1H | 30-50 |
| | 70-90 Compound 1H | 10-30 |
| | 90-99 Compound 1H | 1-10 |
| SC1I | 100 Compound 1I | 0 |
| | 1-99 Compound 1I | 1-99 |
| | 1-10 Compound 1I | 90-99 |
| | 30-50 Compound 1I | 50-70 |
| | 50-70 Compound 1I | 30-50 |
| | 70-90 Compound 1I | 10-30 |
| | 90-99 Compound 1I | 1-10 |
| SC1J | 100 Compound 1J | 0 |
| | 1-99 Compound 1J | 1-99 |
| | 1-10 Compound 1J | 90-99 |
| | 30-50 Compound 1J | 50-70 |
| | 50-70 Compound 1J | 30-50 |
| | 70-90 Compound 1J | 10-30 |
| | 90-99 Compound 1J | 1-10 |
| SC2 | 100 Compound 2 | 0 |
| | 1-99 Compound 2 | 1-99 |
| | 1-10 Compound 2 | 90-99 |
| | 30-50 Compound 2 | 50-70 |
| | 50-70 Compound 2 | 30-50 |
| | 70-90 Compound 2 | 10-30 |
| | 90-99 Compound 2 | 1-10 |
| SC3 | 100 Compound 3 | 0 |
| | 1-99 Compound 3 | 1-99 |
| | 1-10 Compound 3 | 90-99 |
| | 30-50 Compound 3 | 50-70 |
| | 50-70 Compound 3 | 30-50 |
| | 70-90 Compound 3 | 10-30 |
| | 90-99 Compound 3 | 1-10 |
| SC4 | 100 Compound 4 | 0 |
| | 1-99 Compound 4 | 1-99 |
| | 1-10 Compound 4 | 90-99 |
| | 30-50 Compound 4 | 50-70 |
| | 50-70 Compound 4 | 30-50 |
| | 70-90 Compound 4 | 10-30 |
| | 90-99 Compound 4 | 1-10 |
| SC5 | 100 Compound 5 | 0 |
| | 1-99 Compound 5 | 1-99 |
| | 1-10 Compound 5 | 90-99 |
| | 30-50 Compound 5 | 50-70 |
| | 50-70 Compound 5 | 30-50 |
| | 70-90 Compound 5 | 10-30 |
| | 90-99 Compound 5 | 1-10 |
| SC6 | 100 Compound 6 | 0 |
| | 1-99 Compound 6 | 1-99 |
| | 1-10 Compound 6 | 90-99 |
| | 30-50 Compound 6 | 50-70 |
| | 50-70 Compound 6 | 30-50 |
| | 70-90 Compound 6 | 10-30 |
| | 90-99 Compound 6 | 1-10 |

The present invention includes solvent compositions in accordance with the above Table 5 in which the co-solvent is selected from the group consisting of hexafluoroisopropylethylether, hexafluoroisopropylmethylthioether, HFE-7000, HFE-7200, HFE-7100, HFE-7300, HFE-7500, HFE-7600, trans-1,2-dichloroethylene, n-pentane, cyclopentane, ethanol, perfluoro(2-methyl-3-pentanone) (Novec 1230), cis-HFO-1336mzz, trans-HFO-1336mzz, HF-1234yf, HFO-1234ze(E), HFO-1233zd(E) and HFO-1233zd(Z).

### Electrolyte Formulations and Batteries

The present invention also provides electrolyte formulations, and batteries containing electrolyte formulations, which comprise a compound of the present invention, including each of Compositions 1-6, and each of the working fluids in Table 2 above, namely WF1-WF6. In general, the electrolyte formulations comprise: (a) electrolyte; (b) organic solvent for the electrolyte; and (c) additives that are included in the formulation to provide a desired property, or an improvement to a desired property, of the electrolyte formulation and/or of the battery which contains the electrolyte. The compounds of the present inventions, including each of Compositions 1-6, and each of the working fluids in Table 2 above, namely WF1-WF6, can be included in the formulation as a solvent (or co-solvent) for the electrolyte and/or as an additive.

Thus, the present invention provides electrolyte formulations comprising:
(a) a salt, preferably lithium ion salt;
(b) a solvent for the salt, said solvent comprising a compound of the present invention, including each of Compositions 1-6, and each of the working fluids in Table 2 above, namely WF1-WF6, either with or without a co-solvent; and
(c) one or more additives different than the compounds of the present invention.

The present invention also provides electrolyte formulations comprising:
(a) electrolyte, and preferably lithium ion electrolyte;
(b) solvent for the lithium-ion electrolyte; and
(c) an additive comprising a compound of the present invention, including each of Compounds 1-6, either with or without additional additives.

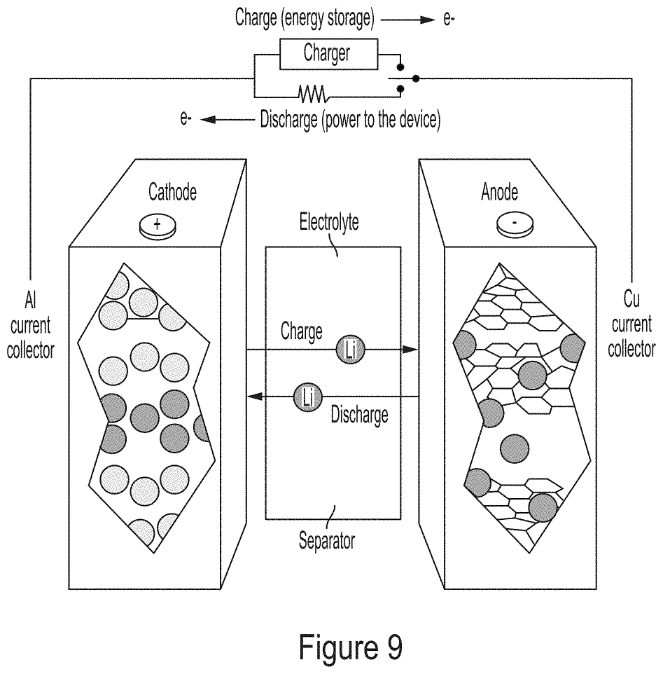

The present invention also provides batteries in general, and rechargeable lithium-ion batteries in particular, which contain an electrolyte formulation containing a compound of the present invention, including each of Compositions 1-6, and each of the working fluids in Table 2 above, namely WF1-WF6. An exemplary rechargeable lithium-ion battery is illustrated in FIG. 9 hereof, which shows a cathode and an anode and electrolyte formulation of the present invention which facilitates the flow of lithium ions between the cathode and the anode.

Although it is contemplated that the present electrolyte formulations may be useful in batteries in general, in preferred embodiments the electrolyte formulation comprises a lithium-ion electrolyte useful in rechargeable batteries. Non-limiting examples of lithium salts that may comprise the electrolyte portion of the formulation include: LiPF6, LiAsF6, LiClO4*LiBF4, LiBC4Og(LiBOB), LiBCO4F,(LiODFB), LiPF3 (C2F5)3(LiFAP), LiBF3(C2F5)LiPF3(C, F5)3(LiFAB), LiN, (CF3SO,), LiN(C,F5SO,), LiCF3S03, LiC(CF3SO,)3, LiPF4(CF3)2, LiPF3(CF3)3, LiPF3(iSO—C3C7)3,LiPF5(iso-C3F7). The overall salt concentration may vary depending on the particular needs of the application, in some embodiments the electrolyte may be present in the formulation in an amount between about 0.3M and about 2.5M or, from about 0.7M to about 1.5M.

### EXAMPLES

### Example 1—Organic Rankine Cycle

This example illustrates that the compositions of the present invention, including each of Compositions 1-6, and each of the working fluids in Table 2 above, namely WF1-WF6, are useful as a working fluid in an Organic Rankine cycle based on a comparison of the estimated thermal efficiency of various working fluids in an organic Rankine cycle. In this example, an ORC system is assumed to contain a condenser, pump, boiler and turbine and the following qualitative results will occur as shown in Table E1 below.

TABLE E1

| | | Process Specifications | | | | | | |
|---|---|---|---|---|---|---|---|---|
| | | | | Boiler | | | | |
| | | Boiler | Critical | Super- | Isentropic | Condensing | Isentropic | Estimated |
| | Working | Temp | Temp | heat | Efficiency | Temp | Efficiency | Thermal |
| | Fluid | (° C.) | (° C.) | (° C.) | (Turbine) | (° C.) | (Pump) | Efficiency |
| Ex. | Compositions | 144 | 225 | 1 | 0.8 | 35 | 0.8 | 15-20% |
| 1A | 1-6, and | | | | | | | |
| | each of the | | | | | | | |
| | working fluids | | | | | | | |
| | in Table 2 | | | | | | | |
| | (WF1-WF6) | | | | | | | |
| | R245fa | 144 | 154 | 1 | 0.8 | 35 | 0.8 | 15.41% |
| | R1233zd(E) | 144 | 166 | 1 | 0.8 | 35 | 0.8 | 15.92% |
| Ex. | Compositions | 210 | 225 | 1 | 0.8 | 35 | 0.8 | 15-20% |
| 1B | 1-6, and | | | | | | | |
| | each of the | | | | | | | |
| | working fluids | | | | | | | |
| | in Table 2 | | | | | | | |
| | (WF1-WF6) | | | | | | | |
| | R245fa | 144 | 154 | 1 | 0.8 | 35 | 0.8 | 15.41% |
| | R1233zd(E) | 156 | 166 | 1 | 0.8 | 35 | 0.8 | 16.66% |

### Example 2—Compositions of the Invention Compared to Novec 7200 in a Heat Exchanger

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Prismatic and pouch cells are often used with cooling plates due to the straight outer faces. Cylindrical cells employ cooling ribbons that are in thermal contact with the outer shell of the cells. Extensive heat generation during charging and discharging of the cells can lead to an increase in temperature that can cause decreasing performance and reduced battery lifetime.

A battery cooling plate set up may be used to provide active cooling to a battery and remove the heat (e.g., to remove heat from the battery of an electric vehicle). In this Example, the performance of fluids of the present invention, including each of Compositions 1-17 and 18A and 3M Novec 7200 is analysed for their ability to provide cooling in single phase heat transfer.

It will be appreciated that the convective heat transfer can occur either by direct contact, i.e., when the battery is immersed in the fluid that may be pumped through the battery enclosure or indirectly, i.e., by using a cooling plate with a combination of convective and conductive heat transfer.

The present example uses a round tube with an internal diameter of 0.55 inches to provide a cooling load of 10246 BTU/h (3 kW). The tube length was 30 ft (9.14 m) with an assumed pressure drop of 2.9 PSI (20 kPa). The fluid temperature was 7.2 C (45F). The internal heat transfer coefficient is determined for turbulent flow. The necessary mass flow rate to remove the cooling load is determined for both fluids. The results of the comparison are shown in the table below. It can be seen in the results that the necessary mass flow rate to remove the generated heat is about or less than for 3M Novec 7200 and that the useful output (I.e., the heat transfer coefficient) is about or higher than 3M Novec 7200.

Heat Transfer and Pressure Drop for Heat Exchanger Set Up

| | Mass Flow | Prandtl | Internal heat |
|---|---|---|---|
| | Rate | Number | transfer coefficient |
| | Ib./s | [—] | BTU/(h-ft²-F.) |
| Compositions 1- | 0.9-1 | 9-11 | 300-350 |
| 6, and each of the | | | |
| working fluids in | | | |
| Table 2 ( WF1- | | | |
| WF6) | | | |
| 3M Novec 7200 | 0.98 | 10.4 | 303.4 |

### Example 3—Secondary AC System

The efficiency of secondary loop air conditioning system, as determined by the estimated coefficient of performance (COP), is evaluated for the use of heat each of Compositions 1-6, and each of the heat transfer compositions in Table 3 (HTC1-HTC6) as a secondary refrigerant with R1234ze(E), R1234yf, and propane as primary refrigerant options. The system is composed of a vapor-compression primary loop and a pumped two-phase secondary loop that are thermally connected by an internal heat exchanger. This internal heat exchanger acted as an evaporator for the primary loop and a condenser for the secondary loop. Using the thermodynamic properties of the primary and secondary refrigerants at the specified conditions of each unit operation, defined in Table E3A, the COP is evaluated relative to the performance of R410A in an air conditioning system (see Table E3B).

TABLE E3A
Operating Conditions

| Operating | T_{cond.} | T_{cond sink} | □T_{SC} | T_{evap.} | T_{evap sink} | □T_{SH} | □_{Isentropic} | □_{Volumetric} | □T_{IHX-SH} | [symbol] T_{IHX-Sat} |
|---|---|---|---|---|---|---|---|---|---|---|
| Conditions | (° C.) | (° C.) | (° C.) | (° C.) | (° C.) | (° C.) | (—) | (—) | (° C.) | (° C.) |
| Basic Cycle | 45 | 35 | −5 | 7 | 27 | +5 | 70% | 100% | N/A | N/A |
| (R410A) | | | | | | | | | | |
| Secondary Cycle | 45 | 35 | −5 | 7 | 27 | [symbol] 0 (flooded) | 70% | 100% | +5 | +5 |
| [symbol] ”/Compositions | | | | | | | | | | |
| 1 [symbol] 6, and each of the | | | | | | | | | | |
| heat transfer | | | | | | | | | | |
| compositions in | | | | | | | | | | |
| Table 3 | | | | | | | | | | |
| (HTC1-HTC6) | | | | | | | | | | |

Nomenclature:
T = Temperature,
□ = Efficiency,
□ = Difference,
SC = Sub-cooling,
SH = Superheat,
IHX = Intermediate Heat Exchanger,
Sat = Saturation [symbol]
indicates data missing or illegible when filed

TABLE E3B
Performance of secondary AC cycle

| Primary | | | | | |
|---|---|---|---|---|---|
| Refrigerant | Secondary | GWP | GWP | | Effi- |
| (“X”) | Refrigerant | Primary | Secondary | Capacity | ciency |
| R410A | | 1924 | | 100% | 100% |
| R1234ze(E) | Compositions | <1 | <1-150 | 100% | 90-95% |
| | 1-6, and | | | | |
| | each of the | | | | |
| | heat transfer | | | | |
| | compositions | | | | |
| | in Table 3 | | | | |
| | (HTC1- | | | | |
| | HTC6) | | | | |
| R1234yf | Compositions | <1 | <1-150 | 100% | 90-95% |
| | 1-6, and | | | | |
| | each of the | | | | |
| | heat transfer | | | | |
| | compositions | | | | |
| | in Table 3 | | | | |
| | (HTC1- | | | | |
| | HTC6) | | | | |
| Propane | Compositions | 3 | <1-150 | 100% | 90-95% |
| | 1-6, and | | | | |
| | each of the | | | | |
| | heat transfer | | | | |
| | compositions | | | | |
| | in Table 3 | | | | |
| | (HTC1- | | | | |
| | HTC6) | | | | |

Table E3B shows the thermodynamic performance of the secondary AC system with different primary refrigerants and using each of Compositions 1-6, and each of the heat transfer compositions in Table 3 (HTC1-HTC6) as secondary refrigerant, with the capacity of the secondary AC system being matched to R410A system in all the cases.

### Example 4—High Temperature Heat Pump Application Using Compositions 1-6, and Each of the Heat Transfer Compositions in Table 3 (HTC1-HTC6)

High temperature heat pumps can utilize waste heat and provide high heat sink temperatures. Compositions 1-6, and each of the heat transfer compositions in Table 3 (HTC1-HTC6) of the present invention each provide efficiency equal to about or superior to R₂₄₅fa over a wide range of condensing temperatures.

Operating Conditions:
Condensing temperature varied between 90° C., 100° C. and 110° C.
Subcooling: 10° C.
Evaporating temperature: 25° C.
Evaporator Superheat: 15° C.
Isentropic efficiency: 65%

TABLE E4
Relative heating COP at varying condensing temperatures

| Fluid | Condensing temperature | | |
|---|---|---|---|
| Fluid | 90° C. | 100° C. | 110° C. |
| R245fa | 100.0% | 100.0% | 100.0% |
| Compositions 1- | 100-105% | 100-105% | 100-105% |
| 6, and each of the | | | |
| heat transfer | | | |
| compositions in | | | |
| Table 3 ( HTC1- | | | |
| HTC6) | | | |

### Example 5—Thermodynamic Performance of a Secondary Loop Medium Temperature Refrigeration System

The efficiency of secondary loop medium temperature refrigeration system, as determined by the estimated coefficient of performance (COP), is evaluated for the use of each of Compositions 1-6, and each of the heat transfer compositions in Table 3 (HTC1-HTC6) as a secondary refrigerant with R1234ze(E), R1234yf, and propane as primary refrigerant options. The system is composed of a vapor-compression primary loop and a pumped two-phase secondary loop that are thermally connected by an internal heat exchanger. This internal heat exchanger acts as an evaporator for the primary loop and a condenser for the secondary loop. The COP was evaluated relative to the performance of R134a in an air conditioning system and the each of Compositions 1-6, and each of the heat transfer compositions in Table 3 (HTC1-HTC6) about matches or is superior to the efficiency of R134a.

### Example 6—Sensible Heat Immersion Cooling Application Using Compositions 1-6, and Each of the Heat Transfer Compositions in Table 3 (HTC1-HTC6)

Batteries of electric vehicles develop heat during operation when charging and discharging. The typical design of vehicle batteries differs between three types: cylindrical cells, pouch cells and prismatic cells. All three types have different considerations in terms of heat transfer due to their shape. Extensive heat generation during charging and discharging of the cells can lead to an increase in temperature that can cause decreasing performance and reduced battery lifetime.

Compositions 1-6, and each of the heat transfer compositions in Table 3 (HTC1-HTC6) of the present invention preferably have low dielectric constants, high dielectric strength, and are non-flammable fluids, which allows for direct cooling of the battery cells that are immersed in each of Compositions 1-6, and each of the heat transfer compositions in Table 3 (HTC1-HTC6).

The present example considers a battery module that consists of 1792 cylindrical battery cells of 18650 type. In one case the battery module is cooled by a 50/50 mixture of water/glycol in a flat tube heat exchanger that is on contact with the battery cells. In the other case the cells are immersed in each of Compositions 1-6, and each of the heat transfer compositions in Table 3 (HTC1-HTC6), i.e., are in direct contact with the fluid. The waste heat for the battery module is 8750 W that is evenly distributed over the total number of cells. The assumptions and operating conditions are listed in Table E5A and E5B.

TABLE E5A
Assumptions for Battery module design and operating conditions

| | | Water/ | Compositions |
|---|---|---|---|
| Parameter | Unit | Glycol | 1-17 and 18A |
| Battery diameter | [mm] | 18.5 | 18.5 |
| Battery gap | [mm] | 3.8 | 1.5 |
| Battery height | [mm] | 65 | |
| Number of batteries | [—] | 1792 | |
| Battery mass | [g] | 49 | |
| Battery specific heat | [J/kgK] | 830 | |
| Total battery module waste heat | [W] | 8750 | |
| Fluid flow rate | [kg/s] | 0.1 | |
| Initial module temperature | [° C.] | 30 | |
| Fluid inlet temperature | [° C.] | 10 | |
| Cooling channel height | [mm] | 30 | n/a |
| Cooling channel width | [mm] | 2.8 | n/a |
| Heat exchanger flat tube wall | [mm] | 0.5 | n/a |
| thickness | | | |
| Heat exchanger flat tube thermal | [W/mK] | 3 | n/a |
| conductivity | | | |
| Heat exchanger flat tube relative | [—] | 0.0003 | n/a |
| surface roughness | | | |

TABLE 5B
Minimum and maximum cell temperatures in battery module

| | Minimum cell | | Maximum cell | |
|---|---|---|---|---|
| | temperature [° C.] | | temperatures [° C.] | |
| | Water/Glycol | Compositions | Water/Glycol | Compositions |
| Time | 50/50 | 1-6 | 50/50 | 1-6 and 6A |
| 0 | 30.0 | 30.0 | 30.0 | 30.0 |
| 100 | 35.8 | 10-40 | 36.8 | 30-40 |
| 200 | 40.3 | 10-40 | 42.0 | 30-40 |
| 300 | 43.6 | 10-45 | 46.0 | 30-45 |
| 400 | 46.1 | 10-45 | 49.2 | 30-50 |
| 500 | 48.0 | 10-45 | 51.7 | 30-50 |
| 600 | 49.5 | 10-50 | 53.6 | 30-55 |
| 700 | 50.5 | 10-50 | 55.1 | 30-55 |
| 800 | 51.4 | 10-50 | 56.3 | 30-55 |
| 900 | 52.0 | 10-50 | 57.2 | 30-55 |

### Example 7—Two Phase Immersion Cooling Application Using Compositions 1-6, and Each of the Heat Transfer Compositions in Table 3 (HTC1-HTC6) in a Data Center

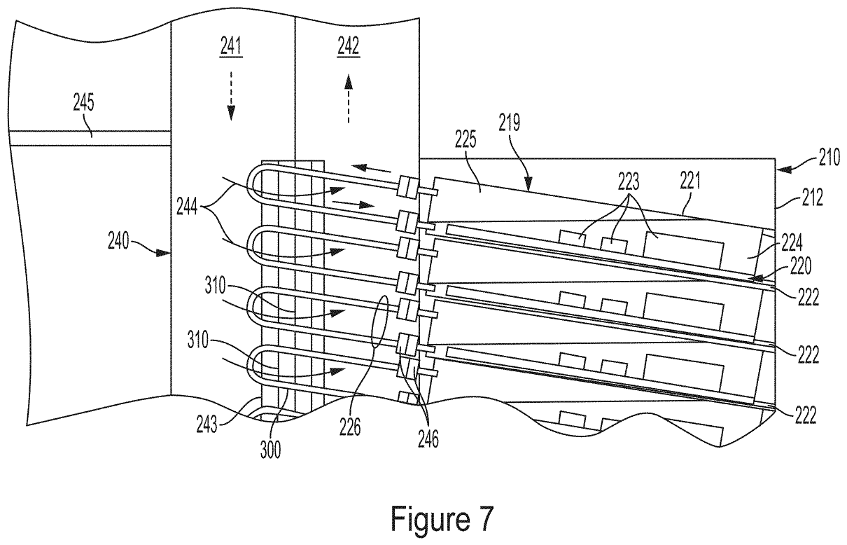

An example of data center cooling is provided, making reference to FIG. 7. A data center, generally denoted 200, includes a plurality of electronic subsystems 220 contained in one or more of electronics racks 210. At least one, and preferably a plurality, and preferably all, of the electronic subsystems 220 are associated with a cooling station 240 that includes (in one embodiment) a vertically extending, liquid-to-air heat exchanger 243 and supply and return ducting 241, 242 for directing a cooling airflow 244 across liquid-to-air heat exchanger 243. A cooling subsystem 219 is associated with at least one, and preferably a plurality, and preferably all, of the multiple electronic subsystems 220. In a preferred embodiment, as shown in FIG. 7, all of the subsystem 220 are associated with the cooling station 240 and a cooling subsystem 219. Each cooling subsystem 219 comprises (in this embodiment) a housing 221 (which preferably is a low-pressure housing) which encloses a respective electronic subsystem 220 comprising a plurality of electronic components 223. The electronic components are in operation as part of the data center and are generating heat as a result of performing their function in the data center. The components include, by way of example, printed circuit boards, microprocessor modules, and memory devices. Each electronic subsystem has, as it is operating, its heat generating components immersed in a thermal management fluid of the present invention 224, including each of Compositions 1-6, and each of the heat transfer compositions in Table 3 (HTC1-HTC6). The fluid 224 boils in typical operation, generating dielectric vapor 225 according to the present invention. In the illustrated embodiment, electronic subsystems 220 are angled by providing upward-sloped support rails 222 within electronics rack 210 to accommodate the electronic subsystems 220 at an angle. Angling of the electronic subsystems as illustrated facilitates buoyancy-driven circulation of vapor 225 between the cooling subsystem 219 and the liquid-to-air heat exchanger 243 of the associated local cooling station 240. However, the excellent results according to the present invention and the present example are achieved equally well when such angling is not used. Multiple coolant loops 226 are coupled in fluid and thermal contact with the liquid-cooled electronic subsystems and a respective portion of liquid-to-air heat exchanger 243. In particular, multiple tubing sections 300 pass through liquid-to-air heat exchanger 243, which in this example includes a plurality of air-cooling fins 310. Vapor 225 is buoyancy-driven from housing 221 to the corresponding tubing section 300 of liquid-to-air heat exchanger 243, where the vapor condenses and is then returned as liquid to the associated liquid-cooled electronics subsystem. Cooling airflow 244 is provided in parallel to the supply ducting 241 of multiple local cooling stations 240 of data center 200, and the heated airflow is exhausted via return ducting 242. The equipment as described herein, but not the fluid of the present invention, is disclosed in US 2013/0019614, which is incorporated herein by reference.

The system as describe above is operated with a thermal management fluid consisting of the present invention, including each of Compositions 1-6, and each of the heat transfer compositions in Table 3 (HTC1-HTC6) and ambient air as the heat sink for the condenser, and this system operates to effectively, efficiently, safely and reliably maintain the electronic components in the most desired operating temperature range while the system is performing its function in the operating data center.

### Example 7—Compositions 1-6, and Each of the Heat Transfer Compositions in Table 3 (HTC1-HTC6) as Solvent or Additive in Lithium-Ion Batteries

Electrolyte solvents and additives play an important role in the performance of lithium-ion batteries (LIB). Compositions 1-6, and each of the working fluids in Table 2 (WF1-WF6) of the present invention is used as a solvent or additive for various electrolyte composition for lithium-ion batteries. Typically, the electrolyte composition comprises dissolved Li salt such as lithium hexafluorophosphate (LiPF6), Lithium bis(fluorosulfonyl)imide (LiFSI), lithium trifluoromethanesulfonate (LiTf), solvents or combination of solvents comprising components such as ethylene carbonate (EC), propylene carbonate (PC), diethylene carbonate (DEC), dimethylene carbonate (DMC) and many other organic carbonates and esters and additives such vinylene carbonate, crown ethers, borates, boronates and many other compounds. The role of solvents in LIB is to serve as the medium for the transfer of charges, which are in the form of ions, between a pair of electrodes. Various modifications of the electrolytes with different components of solvents or additives are also known [For a detailed description, see Kang Xu, “Non-Aqueous Electrolytes for Lithium Based Rechargeable Batteries” Chem. Rev., 2012, 104, 4303-4417]. Compounds of the present invention, including Compositions 1-6, and each of the working fluids in Table 2 (WF1-WF6), can be added as solvents and/or additives to improve the performance of lithium-ion batteries since such the present material have desirable properties such as chemical and thermal stability, desirable dielectric constant and electrochemical window. The present compounds and compositions can be used as a solvent in amounts, for example, ranging from 5-50 wt. % of the solvent, and as additives, in amounts ranging from 0.1 to 5 wt. %, in a variety of electrolyte composition.

### Example 8—Solvent Degreasing

The working fluids of the present invention, including Compositions 1-6, and each of the working fluids in Table 2 (WF1-WF6) is used as the solvent in a degreasing apparatus, as shown for example in FIG. 11, and successfully removes various contaminants, including all of the contaminants mentioned above from a variety of substrates, including all of the substrates mentioned above.

### Example 9—a Representative Method for the Preparation of Compounds of the Family Represented by Formula I

Trifluoroethtyltrifluoromethanesulfonate (CF₃CH₂OSO₂CF₃, 310 ml, 2.15 mol) was mixed with Potassium Carbonate (K₂CO₃, 415.6 g, 3 mole) in oven dried 3 L three necked round bottom flask fitted with mechanical stirrer in the middle neck, and the other neck was fitted with reflux condenser with take-off connected to nitrogen bubbler. Tap water was circulated through reflux condenser, and yet another neck was fitted with thermocouple, and the heterogeneous mixture was stirred, and cooled to 0° C.-5° C. with external ice-water mixture. To the mixture, was added hexafluoro isopropanol ((CF₃)₂CHOH, 455 ml-475 ml, over 4.3 mole) slowly such that temperature of the mixture is maintained under room temperature (RT). The resultant mixture was heated to 78° C.-85° C. using heating mantle/oil bath while maintaining continued stirring for 45-48 h. After this reaction time, the mixture was cooled to room temperature, and 2 L distilled water was added to RB while stirring to dissolve entire solid Potassium Carbonate. The whole reaction mixture was transferred to 4 L separatory funnel, was shaken well. The bottom organic layer was collected in 1 L conical flask, and aqueous top layer was removed. The organic layer was transferred back to the separatory funnel. The organic layer was washed with saturated aqueous potassium carbonate solution by four times (4×500 ml). The organic layer was dried with anhydrous sodium sulphate by thoroughly shaking the mixture in a conical flask fitted with stopper occasionally venting the pressure inside if any, and solid was removed by filtration. Thus, obtained crude product (over 357 g, yield: over 67%) was distilled at atmospheric pressure to get pure product with boiling point: 68-70° C. at 760 mmHg.

The reaction can be shown as follows:

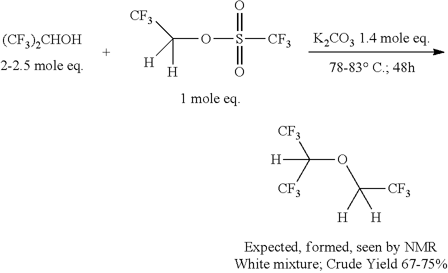 [SMILES: "(CF3)2CHOH" ; O=S(=O)(OCC(F)(F)F)C(F)(F)F ; FC(F)(F)COC(C(F)(F)F)C(F)(F)F]

By product CF₃CH₂OCH₂CF₃ was present was present in the composition in an amount of 0.2% by weight or less. The substantially pure product had a boiling point of from 68-70° C. at 760 mmHg. Following the above procedure, it could be extended to prepare different compounds of the family represent by Formula I.

Alternatively, the ethers of the family represent by Formula I could be synthesized by an alternative route such as Mitsunobu conditions as follows: triphenylphosphine (TPP) and an azodicarboxylate such as diethyl azodicarboxylate (DEAD) or diisopropyl azodicarboxylate (DIAD) are mixed at −10° C. in THF or toluene under nitrogen atmosphere, and the mixture was continued stirring at the same temperature for few minutes. And then two different alcohols are added, and mixture is heated to reflux as needed to form unsymmetrical ethers of the family represent by Formula I.

1. A method of cooling an operating electronic device comprising:
(a) providing a heat transfer composition comprising one or more compounds according to Formula I: 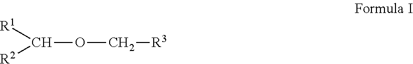
where
R₁, R₂ and R₃ are each independently CₓR′_{(2x+1)-y}H_{y};
each R′ is independently selected from F or Cl and wherein the value of (2x+1)−y is the total number of R′ substituents on the indicated carbon atom(s);
each x is independently equal to or greater than 1 and equal to or less than 6; and
y is equal to or greater than 0 and less than or equal to 2x+1, provided that the total number of R′ present in the compound is 6 or greater, and that the compound has from zero up to two (2) Cl substituents; and
(b) immersing an electronic device or component in said heat transfer composition.

2. The method of claim 1 wherein said heat transfer composition has a global warming potential (GWP) of about 200 or less.

3. The method of claim 1 wherein said heat transfer composition is non-flammable.

4. The method of claim 1 wherein said heat transfer composition has a dielectric constant less than 5 at 20 GHz.

5. The method of claim 1 wherein said heat transfer composition has a dielectric constant less than 4 at 20 GHz.

6. The method of claim 1 wherein said heat transfer composition has a dielectric constant less than 3 at 20 GHz.

7. The method of claim 1 wherein said heat transfer composition has a boiling point of from about 25° C. to about 150° C.

9. The method of claim 1 wherein said heat transfer composition: (i) has a dielectric constant less than 5 at 20 GHz; (ii) has a boiling point of from about 50° C. to about 150° C.; (iii) is non-flammable; and (iv) has an Ames-negative toxicity.

10. The method of claim 9 wherein said heat transfer composition comprises at least about 50% by weight of said one or more compounds according to Formula I.

11. The method of claim 9 wherein said heat transfer composition consists essentially of said one or more compounds according to Formula I.

12. The method of claim 9 wherein said heat transfer composition comprises at least about 50% by weight of a compound according to Formula Ia:
(CF₃)₂CH—O—CH₂CF₃ Formula Ia.

13. The heat transfer methods of claim 9 wherein said electronic device or component comprises one or more of batteries, semiconductor integrated circuits (ICs), electrochemical cells, power transistors, resistors, electroluminescent elements, microprocessors, power control semiconductors, electrical distribution switch gear, power transformers, printed circuit boards, multi-chip modules, packaged or unpackaged semiconductor devices, semiconductor integrated circuits, fuel cells, lasers light emitting diodes (LEDs), electrochemical cells, electrical drive motors and combinations of these.

14. The heat transfer method of claim 13 conducted in an electric vehicle.

15. The heat transfer method of claim 13 conducted in a hybrid gas/electric vehicle.

16. The heat transfer method of claim 13 conducted in a data center.

17. A computer server which is cooled by a method comprising the heat transfer method of claim 1.

18. A crypto mining method comprising the heat transfer method of claim 13.

19. An electric vehicle comprising at least one electronic component or device which is cooled by the method of claim 1.

20. A hybrid gas/electric vehicle comprising at least one electronic component or device which is cooled by the method of claim 1.
